(19) United States
(12) Patent Application Publication
(10) Pub. No.: US 2026/0258430 A1
(43) Pub. Date: Sep. 3, 2026

(54) MOBILE GENOMIC EDITING REAGENTS AND METHODS FOR SCION EDITING

(71) Applicant: Inari Agriculture Technology, Inc., Cambridge, MA (US)

(72) Inventors: Palak KATHIRIA, Belmont, MA (US); Michael Lee NUCCIO, Salem, NH (US)

(73) Assignee: Inari Agriculture Technology, Inc., Cambridge, MA (US)

(21) Appl. No.: 19/163,330

(22) PCT Filed: Mar. 7, 2024
(86) PCT No.: PCT/US2024/018921

§ 371 Date: Sep. 8, 2025

Related U.S. Application Data
(60) Provisional application No. 63/489,708, filed on Mar. 10, 2023.

(51) Int. Cl. C12N 15/82; C12N 9/12; C12N 9/22; C12N 15/10; C12N 15/113

(52) U.S. Cl. CPC C12N 15/8213; C12N 9/1276; C12N 9/226; C12N 15/102; C12N 15/113; C12N 15/8227; C12Y 207/07049; C12N 2310/12; C12N 2310/20; C12N 2310/531; C12N 2830/34

(57) ABSTRACT

The present disclosure provides methods for editing a genomic target in a grafted scion using genomic editing reagents which are fused to meristem transport segments, and which are expressed in the rootstock onto which the scion is grafted. Also provided are plants edited using the methods. Rootstock expressing the genomic editing reagents which are fused to meristem transport segments are also provided.

### CROSS REFERENCE TO RELATED APPLICATIONS

This application claims priority to U.S. Provisional Patent Application No. 63/489,708, filed on Mar. 10, 2023, the entirety of which is incorporated herein by reference.

### REFERENCE TO AN ELECTRONIC SEQUENCE LISTING

The content of the electronic sequence listing (165362001040SEQLIST.xml; Size: 104,719 bytes; and Date of Creation: Mar. 7, 2024) is herein incorporated by reference in its entirety.

### FIELD OF THE INVENTION

In some aspects, the present invention relates to gene editing methods in plants that use Cas enzymes that are fused to a meristem transport segment and can be transported from the root to the meristem of the plant.

### BACKGROUND

Plants do not maintain a population of germ cells throughout their lifetime. Vegetative meristems give rise to floral meristems, which will produce the reproductive organs and gametes. Heritable genome edits in plants therefore require that the edits occur either in the gametes themselves or in the cells of the meristem that will give rise to the gametes. One method of accomplishing this is to deliver a transgene to the genome of the entire plant, which produces genome editing reagents in at least the meristem so as to produce the desired edits.

Meristem nucleic can also be edited without the introduction of a transgene to the meristem itself. RNAs can be targeted to the shoot apical meristem by the addition of meristem transport segments (Kehr and Buhtz J Exp Bot 2008, 59:85-92; Ham and Lucas Annu Rev Plant Biol 2017, 68:173-195; Kehr and Kragler New Phytol 2018, 218:29-40; Kehr et al. Annu Rev Plant Biol 2022, 73:457-474). It has been demonstrated that sequences derived from the Arabidopsis FT transcript are capable of targeting a heterologous, non-mobile RNA to the shoot apical meristem (Li et al. Sci Rep 2011, 1:73; Jackson and Hong Front Plant Sci 2012: doi: 10.3389/fpls.2012.00127). Similar results have been shown for sequences derived from some transfer-RNAs (Zhang et al. Plant Cell 2016, 28:1237-1249). Meristem transport segments have been fused to genome editing reagent transcripts to enable them to move from one part of the plant, such as the leaf or root, to the shoot apical meristem (Doyle et al. BioRxiv, 2019:805036). Thus, the RNA encoding genome editing reagents is produced in one part of the plant, loaded into the phloem, and transported to the shoot apical meristem where it is translated and assembled into mature ribonucleoproteins (RNPs) to perform genome editing in meristem nuclei which will eventually form the plant reproductive structures. Heritable edits are the result. However, this method is still limited to species that are amenable to transformation.

A recent method to introduce germline edits is to target genome editing reagents, including an RNA-guided nuclease and at least one corresponding guide RNA, to the shoot apical meristem (Imai et al. Plant Biotechnol 2020, 37(2): 171-176). This can be achieved through constitutive expression of the nuclear-localized CRISPR Cas nuclease using highly active promoters like those based on ubiquitin genes or CaMV 35S, and expression of the guide RNA(s) from RNA polymerase III promoters (Hassan et al. Trends Plant Sci 2021, 26:1133-1152). Guide RNAs can be expressed from a constitutive RNA polymerase II promoter if flanked by self-cleaving ribozymes that remove 5′- and 3′-flanking sequence (Tang et al. Plant Biotechnol J 2019, 17:1431-1445). It is also possible to directly express both the CRISPR Cas nuclease and guide RNAs in the shoot apical meristem using promoters that are highly active in those cells alone (Jackson et al. Development 1994, 120:405-413). All these approaches require direct expression of the genome editing reagents in the cells to be edited, which limits direct editing to germplasm that can be transformed using routine methods such as Agrobacterium (Altpeter et al. Plant Cell 2016, 28:1510-1520) or biolistics (Kikkert et al. Methods Mol Biol Clifton NJ 2005, 286:61-78). Species of plants that are difficult to transform are difficult to edit in this manner, and the introduction of a transgene in order to make the edits requires additional screening and/or breeding to later remove the transgene or ensure that it does not cause unwanted effects by disrupting an existing genomic element.

Grafting is a plant procedure in which one plant part from a first genetic donor is functionally fused with a second plant part from a second, and distinct, genetic donor (Bezdicek et al. Agron J 1972, 64:558-558; Cao et al. Crop Pasture Sci 2019, 70:585-594). A common use for grafting is to join a rootstock that confers a trait beneficial to growth and/or survival (e.g. robust disease resistance) with a shoot (or scion) that produces high quality fruit. Grafting has been historically quite successful in dicot species and some trees but has only been recently demonstrated in monocots (Reeves et al. Nature 2022, 602:280-286). A hallmark of successful grafting is vascular mobility and transmission through a graft junction. Materials loaded into the plant vascular system in the rootstock can be transmitted through the graft junction to the plant scion, and vice versa.

Genome editing of commercial crops is limited by the well-known general recalcitrance to transformation of the elite materials. Editing experimental materials and crossing the edits into elite germplasm takes many generations, and the eventual edited phenotype is not predictable. A simple “one step” process for making genome-edited seeds of elite materials would save time and money, enlarging the capacity of a plant editing pipeline to make edits and observe phenotypes in genetic backgrounds of commercial relevance.

CRISPR technology for editing the genes of eukaryotes is disclosed in U.S. Patent Application Publications 2016/0138008A1 (now U.S. Pat. No. 10,227,11) and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 (Cas12a) endonucleases and corresponding guide RNAs and PAM sites are disclosed in U.S. Pat. No. 9,790,490 and U.S. patent application Ser. No. 15/566,528 (national phase of PCT Application PCT/EP2016/058442, published as WO 2016/166340), now published as U.S. Patent Application Publication 2018/0282713. Other CRISPR nucleases useful for editing genomes include C2c1 and C2c3 (see Shmakov et al. Mol. Cell 2015, 60:385-397) and CasX and CasY (see Burstein et al. Nature 2016, doi: 10.1038/nature21059). Plant RNA promoters for expressing CRISPR guide RNA and plant codon-optimized CRISPR Cas9 endonuclease are disclosed in U.S. patent application Ser. No. 15/120,110, published as U.S. Patent Application Publication 2017/0166912, national phase application claiming priority to International Patent Application PCT/US2015/018104 (published as WO 2015/131101 and claiming priority to U.S. Provisional Patent Application 61/945,700). Methods of using CRISPR technology for genome editing in plants are disclosed in in U.S. Patent Application Publications U.S. 2015/0082478A1 and U.S. 2015/0059010A1 and in International Patent Application PCT/US2015/038767 A1 (published as WO 2016/007347, claiming priority to U.S. Provisional Patent Application 62/023,246, with U.S. National Phase application U.S. Ser. No. 15/325,116, now published as U.S. Patent Application Publication 2017/0306349).

A need exists in the art for a method of introducing heritable edits to the meristem of a plant without the introduction of a transgene to the meristem genome and with the possibility of editing a multitude of species.

### SUMMARY

The present disclosure provides methods of editing a genomic target in a meristem or a plant or grafted scion comprising nucleic acid encoding a Cas nuclease and in some embodiments at least one gRNA, fused to a meristem transport segment (MTS), and edited plants therefrom.

Provided herein is a method of editing a genomic target in a scion comprising grafting the scion onto a rootstock comprising nucleic acid encoding a Cas9 nickase or Cas12 nuclease, and nucleic acid encoding a guide RNA for the Cas9 nickase or Cas12 nuclease, wherein the nucleic acid encoding the guide RNA and the nucleic acid encoding the Cas9 nickase or the Cas12 nuclease are fused to nucleic acid encoding a meristem transport segment (MTS).

In some embodiments, the Cas9 nickase or Cas12 nuclease is associated with a reverse transcriptase. In some embodiments, the Cas9 nickase or Cas12 nuclease is fused to the reverse transcriptase. In some embodiments, the guide RNA comprises at its 3′ end a priming site and an edit to be incorporated into the genomic target. In some embodiments, the Cas12 nuclease is a Cas12 nickase. In some embodiments, the Cas12 nickase comprises mutation in one more nuclease active sites.

In some embodiments, RNA encoding the Cas9 nickase or Cas12 nuclease and the guide RNA are transported from the rootstock to the scion by the plant vascular system. In some embodiments, RNA encoding the Cas9 nickase or Cas12 nuclease and the guide RNA are transported from the rootstock to the scion through the phloem.

In some embodiments, RNA encoding the Cas9 nickase or Cas12 nuclease is translated in the scion.

In some embodiments, a meristem of the scion is edited.

In some embodiments, the nucleic acid encoding the Cas9 nickase or Cas12 nuclease and the nucleic acid encoding the guide RNA are provided in the same vector. In some embodiments, the nucleic acid encoding the Cas9 nickase or Cas12 nuclease and the nucleic acid encoding the guide RNA are provided in different vectors. In some embodiments, the vector is a viral vector. In some embodiments, the vector is a T-DNA vector. In some embodiments, the vector is a viral vector or a T-DNA vector.

In some embodiments, the scion and the rootstock are different plant species. In some embodiments, the scion and the rootstock are the same plant species. In some embodiments, the scion and/or rootstock is a dicot. In some embodiments, the scion and/or rootstock is a monocot. In some embodiments, the scion is soy, canola, alfalfa, corn, oat, sorghum, sugarcane, banana, or wheat.

In some embodiments, the meristem transport segment (MTS) comprises a Flowering Locus T (FT)-derived sequence, a tRNA like sequence (TLS), a meristem transport component (MTC); or an RNA hairpin comprising a first stem of 8 to 12 nucleotides, at least one variable bulge, a second stem of 4 to 7 nucleotides, and a variable loop. In some embodiments, the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24. In some embodiments, the TLS comprises the nucleotide sequence set forth in SEQ ID NO: 29 or 30.

In some embodiments, the nucleic acid encoding the MTS is located 3′ of the nucleic acid encoding the Cas9 nickase or Cas12 nuclease and/or 3′ of the nucleic acid encoding the guide RNA. In some embodiments, the nucleic acid encoding the MTS is located 5′ of the nucleic acid encoding the Cas9 nickase or Cas12 nuclease and/or 5′ of the nucleic acid encoding the guide RNA.

In some embodiments, the nucleic acid encoding the Cas9 nickase or Cas12 nuclease is operably linked to a promoter. In some embodiments, the promoter is active in roots and/or phloem companion cells. In some embodiments, the promoter is the promoter of a gene selected from the group consisting of Arabidopsis WRKY6, chickpea WRKY31, carrot MYB113, corn GLU1, strawberry RB7-type TIP-2, and banana TIP2-2, or the promoter of an orthologous gene thereof. In some embodiments, the promoter is selected from the group consisting of a promoter from a Flowering Locus T (FT) gene, a promoter from a Fabaceaen FOR1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter.

In some embodiments, the nucleic acid encoding the Cas9 nickase or Cas12 nuclease is codon-optimized for expression in dicots. In some embodiments, the nucleic acid encoding the Cas9 nickase or Cas12 nuclease is codon-optimized for expression in monocots. In some embodiments, the nucleic acid encoding the Cas9 nickase or Cas12 nuclease is codon-optimized for expression in corn, soy, or wheat.

In some embodiments, the nucleic acid encoding the guide RNA is operably linked to a promoter. In some embodiments, the promoter is an RNA polymerase II promoter or an RNA polymerase III promoter. In some embodiments, the RNA polymerase II promoter or RNA polymerase III promoter is endogenous to the species of the rootstock.

In some embodiments, the nucleic acid encoding the guide RNA and the MTS is located between two ribozyme sequences. In some embodiments, each of the ribozyme sequences is independently selected from the group consisting of a hammerhead ribozyme sequence, a HDV ribozyme sequence, a Csy4 sequence, and a tRNA processing enzyme sequence. In some embodiments, the nucleic acid encoding the guide RNA and the MTS further comprises a hammerhead ribozyme sequence 5′ to the nucleic acid encoding the guide RNA and the MTS, and a HDV ribozyme 3′ to the nucleic acid encoding the guide RNA and the MTS.

In some embodiments, the nucleic acid encoding the guide RNA and the MTS further comprises a terminator. In some embodiments, the terminator is a U6 terminator.

In some embodiments, the rootstock comprises nucleic acid encoding two or more, three or more, four or more, or five or more guide RNAs. In some embodiments, the nucleic acid encoding each of the two or more, three or more, four or more, or five or more guide RNAs is joined to an MTS.

In some embodiments, the Cas12 nuclease is selected from the group consisting of Cas12a (Cpf1), Cas12e (CasX), Cas12d (CasY), Cas12h, Cas12i, and Cas12j.

In some embodiments, the rootstock further comprises nucleic acid encoding a detectable marker fused to a nucleic acid encoding an MTS.

In some embodiments, the method further comprises retrieving a progeny of the scion, wherein the progeny has an altered genome.

In some embodiments, two or more guide RNAs are encoded by a single precursor RNA. In some embodiments, the two or more guide RNAs are each flanked by a direct repeat.

In other aspects, provided herein is an edited plant produced by the method of any one of the preceding embodiments. In other aspects, provided herein is an edited plant genome of a plant produced by the method of any one of the preceding embodiments. In other aspects, provided herein is a non-regenerable plant cell, tissue, or plant part of a plant produced by the method of any one of the preceding embodiments.

Provided herein is a rootstock comprising nucleic acid encoding a Cas9 nickase or Cas12 nuclease and nucleic acid encoding a guide RNA for the Cas9 nickase or Cas12 nuclease, wherein the nucleic acid encoding the guide RNA and the nucleic acid encoding the Cas9 nickase or Cas12 nuclease are fused to nucleic acid encoding a meristem transport segment (MTS).

In some embodiments, the meristem transport segment (MTS) comprises a Flowering Locus T (FT)-derived sequence, a tRNA like sequence (TLS), a meristem transport component (MTC); or an RNA hairpin comprising a first stem of 8 to 12 nucleotides, at least one variable bulge, a second stem of 4 to 7 nucleotides, and a variable loop. In some embodiments, the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24. In some embodiments, the TLS comprises the nucleotide sequence set forth in SEQ ID NO: 29 or 30.

In some embodiments, the nucleic acid encoding the MTS is located 3′ of the nucleic acid encoding the Cas9 nickase or Cas12 nuclease and/or 3′ of the nucleic acid encoding the guide RNA. In some embodiments, the nucleic acid encoding the MTS is located 5′ of the nucleic acid encoding the Cas9 nickase or Cas12 nuclease and/or 5′ of the nucleic acid encoding the guide RNA.

In some embodiments, the nucleic acid encoding the Cas9 nickase or Cas12 nuclease is operably linked to a promoter. In some embodiments, the promoter is active in roots and/or phloem companion cells. In some embodiments, the promoter is the promoter of a gene selected from the group consisting of Arabidopsis WRKY6, chickpea WRKY31, carrot MYB113, corn GLU1, strawberry RB7-type TIP-2, and banana TIP2-2, or the promoter of an orthologous gene thereof. In some embodiments, the promoter is selected from the group consisting of a promoter from a Flowering Locus T (FT) gene, a promoter from a Fabaceaen FOR1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter.

In some embodiments, the nucleic acid encoding the Cas9 nickase or Cas12 nuclease is codon-optimized for expression in dicots. In some embodiments, the nucleic acid encoding the Cas9 nickase or Cas12 nuclease is codon-optimized for expression in monocots. In some embodiments, the nucleic acid encoding the Cas9 nickase or Cas12 nuclease is codon-optimized for expression in corn, soy, or wheat.

In some embodiments, the nucleic acid encoding the guide RNA is operably linked to a promoter. In some embodiments, the promoter is an RNA polymerase II promoter or an RNA polymerase III promoter. In some embodiments, the RNA polymerase II promoter or RNA polymerase III promoter is endogenous to the species of the rootstock.

In some embodiments, the nucleic acid encoding the guide RNA and the MTS is located between two ribozyme sequences. In some embodiments, each of the ribozyme sequences is independently selected from the group consisting of a hammerhead ribozyme sequence, a HDV ribozyme sequence, a Csy4 sequence, and a tRNA processing enzyme sequence. In some embodiments, the nucleic acid encoding the guide RNA and the MTS further comprises a hammerhead ribozyme sequence 5′ to the nucleic acid encoding the guide RNA and the MTS, and a HDV ribozyme 3′ to the nucleic acid encoding the guide RNA and the MTS.

In some embodiments, the nucleic acid encoding the guide RNA and the MTS further comprises a terminator. In some embodiments, the terminator is a U6 terminator.

In some embodiments, the rootstock comprises nucleic acid encoding two or more, three or more, four or more, or five or more guide RNAs. In some embodiments, the nucleic acid encoding each of the two or more, three or more, four or more, or five or more guide RNAs is joined to an MTS.

In some embodiments, the Cas12 nuclease is selected from the group consisting of Cas12a (Cpf1), Cas12e (CasX), Cas12d (CasY), Cas12h, Cas12i, and Cas12j.

In some embodiments the vector is a viral vector or a T-DNA vector.

In other aspects, provided herein is a method of editing a genomic target in a scion, comprising grafting the scion onto a rootstock expressing a Cas nuclease, wherein the rootstock comprises nucleic acid encoding the Cas nuclease fused to a meristem transport segment (MTS); and delivering a guide RNA for the Cas nuclease to the scion. In some embodiments, the method further comprises transforming the rootstock with nucleic acid encoding the Cas nuclease prior to grafting. In some embodiments, the scion comprises a leaf, a shoot, a stem, and/or a meristem.

In some aspects, provided herein is a method of editing a genomic target in a meristem of a plant, comprising transforming the root of the plant with nucleic acid encoding a Cas nuclease; and delivering a guide RNA for the Cas nuclease to a leaf, a shoot, a stem, and/or a meristem of a the plant, wherein the nucleic acid encoding the Cas nuclease is fused to a meristem transport segment (MTS).

In some embodiments, the guide RNA is fused to a meristem transport segment (MTS).

In some embodiments, delivery of the guide RNA comprises spraying a the guide RNA.

In some embodiments, delivery of the guide RNA comprises rubbing a composition comprising the guide RNA onto the leaves, shoot, stem, and/or meristem.

In some embodiments, delivery of the guide RNA comprises injecting a composition comprising the guide RNA into the stem.

In some embodiments, delivery of the guide RNA comprises leaf infiltration of a composition comprising the guide RNA into the leaf. In some embodiments, the leaf infiltration comprises forced infiltration using a needle-less syringe or vacuum pump.

In some embodiments, the composition comprising the guide RNA comprises a nuclease inhibitor. In some embodiments, the nuclease inhibitor comprises an RNase inhibitor.

In some embodiments, application comprises biolistic transformation of nucleic acid encoding the guide RNA into the leaf, shoot, shoot, stem, and/or meristem. In some embodiments, the biolistic transformation comprises transformation of circular DNA encoding the guide RNA.

In some embodiments, RNA encoding the Cas nuclease and/or the guide RNA is transported by plant vascular system. In some embodiments, RNA encoding the Cas nuclease and/or the guide RNA is transported to the scion through the xylem or the phloem. In some embodiments, RNA encoding the Cas nuclease and/or the guide RNA is transported to the meristem. In some embodiments, RNA encoding the Cas nuclease is translated in the meristem.

In some embodiments, the meristem is edited.

In some embodiments, two or more guide RNAs are encoded by a single precursor RNA. In some embodiments, the two or more guide RNAs are each flanked by a direct repeat.

In some embodiments, the scion and the rootstock are different plant species. In some embodiments, the scion and the rootstock are the same plant species. In some embodiments, the scion and/or rootstock is a dicot. In some embodiments, the plant is a dicot. In some embodiments, the scion and/or rootstock is a monocot. In some embodiments, the plant is a monocot. In some embodiments, the rootstock, scion, and/or plant is soy, canola, alfalfa, corn, oat, sorghum, sugarcane, banana, or wheat.

In some embodiments, the MTS is a Flowering Locus T (FT)-derived sequence, a tRNA like sequence (TLS), a meristem transport component (MTC); or an RNA hairpin comprising a first stem of 8 to 12 nucleotides, at least one variable bulge, a second stem of 4 to 7 nucleotides, and a variable loop. In some embodiments, the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24. In some embodiments, the TLS comprises the nucleotide sequence set forth in SEQ ID NO: 29 or 30.

In some embodiments, the nucleic acid encoding the MTS is located 3′ of the nucleic acid encoding the Cas nuclease. In some embodiments, the nucleic acid encoding the MTS is located 5′ of the nucleic acid encoding the Cas nuclease.

In some embodiments, the nucleic acid encoding the Cas enzyme is operably linked to a promoter. In some embodiments, the promoter is active in roots and/or phloem companion cells. In some embodiments, the promoter is the promoter of a gene selected from the group consisting of Arabidopsis WRKY6, chickpea WRKY31, carrot MYB113, corn GLU1, strawberry RB7-type TIP-2, and banana TIP2-2, or the promoter of an orthologous gene thereof. In some embodiments, the promoter is selected from the group consisting of a promoter from a Flowering Locus T (FT) gene, a promoter from a Fabaceaen FOR1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter. In some embodiments, the promoter is a constitutive promoter. In some embodiments, the constitutive promoter is a ubiquitin promoter.

In some embodiments, the nucleic acid encoding the Cas nuclease is codon-optimized for expression in dicots. In some embodiments, the nucleic acid encoding the Cas nuclease is codon-optimized for expression in monocots. In some embodiments, the nucleic acid encoding the Cas nuclease is codon-optimized for expression in corn, soy, or wheat.

In some embodiments, the method comprises applying two or more, three or more, four or more, or five or more guide RNAs. In some embodiments, the two or more, three or more, four or more, or five or more guide RNAs are each joined to an MTS.

In some embodiments, the Cas nuclease is selected from the group consisting of Cas9, Cas12a (Cpf1), Cas12e (CasX), Cas12d (CasY), C2c1, C2c2, C2c3, Cas12h, Cas12i, and Cas12j.

In some embodiments, the Cas nuclease is associated with a reverse transcriptase. In some embodiments, the Cas nuclease is fused to the reverse transcriptase. In some embodiments, the guide RNA comprises at its 3′ end a priming site and an edit to be incorporated into the genomic target. In some embodiments, the Cas nuclease is a Cas nickase. In some embodiments, the Cas nickase is a Cas9 nickase or a Cas12 nickase. In some embodiments, the Cas nickase comprises mutation in one or more nuclease active sites.

In some embodiments, the plant further comprises nucleic acid encoding a detectable marker fused to a nucleic acid encoding an MTS. In some embodiments, the guide RNA comprises a 5-methylcytosine group.

In some embodiments, the nucleic acid encoding the guide RNA and the MTS is located between two ribozyme sequences. In some embodiments, each of the ribozyme sequences is independently selected from the group consisting of a hammerhead ribozyme sequence, a HDV ribozyme sequence, a Csy4 sequence, and a tRNA processing enzyme sequence. In some embodiments, the nucleic acid encoding the guide RNA and the MTS further comprises a hammerhead ribozyme sequence 5′ to the nucleic acid encoding the guide RNA and the MTS, and a HDV ribozyme 3′ to the nucleic acid encoding the guide RNA and the MTS.

In some embodiments, the nucleic acid encoding the guide RNA and the MTS further comprises a terminator. In some embodiments, the terminator is a U6 terminator.

In some embodiments, the method further comprises retrieving a progeny of the scion or the plant, wherein the progeny has an altered genome.

In some embodiments the guide RNA further comprises (a) one or more modified nucleotides within five nucleotides from the 5′ end of the guide RNA; or (b) one or more modified nucleotides within five nucleotides from the 3′ end of the guide RNA; or (c) both (a) and (b); wherein the one or more modified nucleotides has a modification to a phosphodiester linkage, a sugar, or both a phosphodiester linkage and a sugar. In some embodiments, each of the one or more modified nucleotides is independently selected from the group consisting of a 2′-O-methyl nucleotide, a 2′-O-methyl-3′-phosphorothioate nucleotide, a 2′-O-methyl-3′-phosphonoacetate nucleotide, and a 2′-O-methyl-3′-phosphonothioacetate nucleotide. In some embodiments, the one or more modified nucleotide comprises a modified internucleotide linkage or a modified terminal phosphate group selected from the group consisting of an alkylphosphonate, a phosphonocarboxylate, a phosphonoacetate, a boranophosphonate, a phosphorothioate, a phosphonothioacetate, and a phosphorodithioate group.

In other aspects, provided herein is an edited plant produced by the method of any one of the preceding embodiments. In other aspects, provided herein is an edited plant genome of a plant produced by the method of any one of the preceding embodiments. In other aspects, provided herein is a non-regenerable plant cell, tissue, or plant part of a plant produced by the method of any one of the preceding embodiments.

In other aspects, provided herein is a method of editing a genomic target in a plant meristem, comprising delivering a guide RNA for a Cas nuclease to a plant root, wherein the guide RNA is fused to a meristem transport segment (MTS), wherein the plant comprises nucleic acid encoding the Cas nuclease. In some embodiments, the Cas nuclease is constitutively expressed in the plant.

In some embodiments, the plant comprises a rootstock and a scion grafted onto the rootstock. In some embodiments, the Cas nuclease is expressed in the rootstock.

In some embodiments, the guide RNA is delivered to the plant root by incubating the root with a composition comprising the guide RNA.

In some embodiments, the guide RNA is delivered to the plant root by an Agrobacterium rhizogenes transformation. In some embodiments, the Agrobacterium rhizogenes transformation produces transgenic hairy roots.

In some embodiments, the guide RNA is delivered to the plant root by injecting a composition comprising the guide RNA into the root.

In some embodiments, the composition comprising the guide RNA comprises a nuclease inhibitor, optionally, wherein the nuclease inhibitor is an RNase inhibitor.

In some embodiments, the guide RNA comprises a 5-methylcytosine group.

In some embodiments, the nucleic acid encoding the Cas nuclease is fused to an MTS.

In some embodiments, RNA encoding the Cas nuclease and/or the guide RNA is transported by plant vascular system. In some embodiments, RNA encoding the Cas nuclease and/or the guide RNA is transported through the xylem or the phloem. In some embodiments, RNA encoding the Cas nuclease and/or the guide RNA is transported to the meristem, wherein the Cas nuclease and/or the guide RNA is translated in the meristem.

In some embodiments, a genomic target within the meristem is edited.

In some embodiments, the scion and the rootstock are different plant species. In some embodiments, the scion and the rootstock are the same plant species. In some embodiments, the scion and/or rootstock is a dicot. In some embodiments, the plant is a dicot. In some embodiments, the scion and/or rootstock is a monocot. In some embodiments, the plant is a monocot. In some embodiments, the rootstock, scion, and/or plant is soy, canola, alfalfa, corn, oat, sorghum, sugarcane, banana, or wheat.

In some embodiments, the MTS is a Flowering Locus T (FT)-derived sequence, a tRNA like sequence, a meristem transport component (MTC); or an RNA hairpin comprising a first stem of 8 to 12 nucleotides, at least one variable bulge, a second stem of 4 to 7 nucleotides, and a variable loop. In some embodiments, the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24. In some embodiments, the TLS comprises the nucleotide sequence set forth in SEQ ID NO: 29 or 30.

In some embodiments, the nucleic acid encoding the MTS is located 3′ of the nucleic acid encoding the Cas nuclease and/or 3′ of the guide RNA. In some embodiments, the nucleic acid encoding the MTS is located 5′ of the nucleic acid encoding the Cas nuclease and/or 5′ of the guide RNA.

In some embodiments, the nucleic acid encoding the Cas enzyme is operably linked to a promoter. In some embodiments, the promoter is active in roots and/or phloem companion cells. In some embodiments, the promoter is the promoter of a gene selected from the group consisting of Arabidopsis WRKY6, chickpea WRKY31, carrot MYB113, corn GLU1, strawberry RB7-type TIP-2, and banana TIP2-2, or the promoter of an orthologous gene thereof. In some embodiments, the promoter is selected from the group consisting of a promoter from a Flowering Locus T (FT) gene, a promoter from a Fabaceaen FOR1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter.

In some embodiments, the nucleic acid encoding the Cas nuclease is codon-optimized for expression in dicots. In some embodiments, the nucleic acid encoding the Cas nuclease is codon-optimized for expression in monocots. In some embodiments, the nucleic acid encoding the Cas nuclease is codon-optimized for expression in corn, soy, or wheat.

In some embodiments, the method comprises applying two or more, three or more, four or more, or five or more guide RNAs. In some embodiments, the two or more, three or more, four or more, or five or more guide RNAs are each joined to an MTS.

In some embodiments, the Cas nuclease is selected from the group consisting of Cas9, Cas12a (Cpf1), Cas12e (CasX), Cas12d (CasY), C2c1, C2c2, C2c3, Cas12h, Cas12i, and Cas12j.

In some embodiments, the Cas nuclease is associated with a reverse transcriptase. In some embodiments, the Cas nuclease is fused to the reverse transcriptase. In some embodiments, the guide RNA comprises at its 3′ end a priming site and an edit to be incorporated into the genomic target. In some embodiments, the Cas nuclease is a Cas nickase. In some embodiments, the Cas nickase is a Cas9 nickase or a Cas12 nickase. In some embodiments, the Cas nickase comprises mutation in one or more nuclease active sites.

In some embodiments, the plant further comprises nucleic acid encoding a detectable marker fused to a nucleic acid encoding an MTS.

In some embodiments, the nucleic acid encoding the guide RNA and the MTS is located between two ribozyme sequence. In some embodiments, each of the ribozyme sequences is independently selected from the group consisting of a hammerhead ribozyme sequence, a HDV ribozyme sequence, a Csy4 sequence, and a tRNA processing enzyme sequence. In some embodiments, the nucleic acid encoding the guide RNA and the MTS further comprises a hammerhead ribozyme sequence 5′ to the nucleic acid encoding the guide RNA and the MTS, and a HDV ribozyme 3′ to the nucleic acid encoding the guide RNA and the MTS.

In some embodiments, the nucleic acid encoding the guide RNA and the MTS further comprises a terminator. In some embodiments, the terminator is a U6 terminator.

In some embodiments, the method further comprises retrieving a progeny of the plant, wherein the progeny has an altered genome.

In some embodiments the guide RNA further comprises (a) one or more modified nucleotides within five nucleotides from the 5′ end of the guide RNA; or (b) one or more modified nucleotides within five nucleotides from the 3′ end of the guide RNA; or (c) both (a) and (b); wherein the one or more modified nucleotides has a modification to a phosphodiester linkage, a sugar, or both a phosphodiester linkage and a sugar. In some embodiments, each of the one or more modified nucleotides is independently selected from the group consisting of a 2′-O-methyl nucleotide, a 2′-O-methyl-3′-phosphorothioate nucleotide, a 2′-O-methyl-3′-phosphonoacetate nucleotide, and a 2′-O-methyl-3′-phosphonothioacetate nucleotide. In some embodiments, the one or more modified nucleotide comprises a modified internucleotide linkage or a modified terminal phosphate group selected from the group consisting of an alkylphosphonate, a phosphonocarboxylate, a phosphonoacetate, a boranophosphonate, a phosphorothioate, a phosphonothioacetate, and a phosphorodithioate group.

In other aspects, provided herein is an edited plant produced by the method of any one of the preceding embodiments. In other aspects, provided herein is an edited plant genome of a plant produced by the method of any one of the preceding embodiments. In other aspects, provided herein is a non-regenerable plant cell, tissue, or plant part of a plant produced by the method of any one of the preceding embodiments.

### DETAILED DESCRIPTION

All references cited herein are hereby incorporated by reference in their entirety.

### Definitions

The phrase “allelic variant” as used herein refers to a polynucleotide or polypeptide sequence variant that occurs in a different strain, variety, or isolate of a given organism.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “codon optimization” refers to the process of modifying a nucleic acid sequence for use in a desired host kingdom, phylum, class, order, family, genus, or species, by replacing at least one codon of the nucleic acid with codons that are more frequently used in the genes of the desired host kingdom, phylum, class, order, family, genus, or species, without alteration of the amino acid sequence encoded by the nucleic acid.

As used herein, the term “complementary” refers to sequences with at least sufficient complementarity to permit enough base-paring for two nucleic acids to hybridize (for example, for a tether to hybridize with or bind to a gRNA or donor DNA), which in some examples may be under typical physiological conditions for the cell. In some examples, the oligonucleotide or polynucleotide is at least 80% complementary to the target, for example, at least 85%, 90%, 95%, 96%, 97%, 98%, 99%, or 100% complementary to the target.

As used herein, the term “complex” refers to two or more associated components, such as two or more associated nucleic acids and/or proteins. A complex may include two or more covalently linked nucleic acids and/or proteins, two or more non-covalently linked nucleic acids and/or proteins, or a combination thereof.

As used herein, the terms “comprise,” comprises, “comprising,” “include,” “includes,” and “including” can be interchanged and are to be construed as at least having the features to which they refer while not excluding any additional unspecified features.

As used herein, the term “CRISPR-Cas nuclease” and “Cas nuclease” are used interchangeably herein to refer to the same grouping of RNA directed nucleases.

As used herein, the term “engineered” means artificial, synthetic, or not occurring in nature. For example, a polynucleotide that includes two DNA sequences that are heterologous to each other can be engineered or synthesized by recombinant nucleic acid techniques.

As used herein, the terms “a graft,” “to graft,” and “grafting” refer to the technique wherein two plants are joined by their vasculature such that they fuse to form a single grafted plant. The plant that maintains or will maintain the root system after grafting is referred to herein as the “rootstock”. The plant grafted onto the rootstock is referred to herein as the “shoot”, “plant scion” or “scion”. Grafting includes “micrografting” (Peña et al. Plant Cell Rep 1995, 14:616-619; CN105519434A; CN110178564A), “minigrafting” (Marques et al. Sci Hortic 2011, 129:176-182), and other forms of grafting known to those in the art.

As used herein, the term “heterograft” refers to a graft between a rootstock and a scion of different species.

As used herein, the term “homograft” refers to a graft between a rootstock and a scion of the same species.

As used herein, the terms “include,” “includes,” and “including” are to be construed as at least having the features to which they refer while not excluding any additional unspecified features.

As used herein, the phrase “meristem transport segment” or “MTS” refers to an RNA tag that, when fused to another RNA molecule, results in delivery of the RNA fusion molecule to the meristem of the plant.

As used herein, the term “mobile” refers to the ability of a molecule or a collection of molecules to move within the plant. A fusion of a nucleic acid encoding a Cas nuclease and a meristem transport segment (MTS) results in a mobile Cas, which is capable of being transported through the plant vascular system to the meristem of the plant, including through a graft junction. Similarly, a fusion of an RNA molecule and a meristem transport segment (MTS) results in a “mobile RNA”, which is capable of being transported through the plant vascular system to the meristem of the plant, including through a graft junction.

As used herein, the phrase “operably linked” or “fused” refers to a juxtaposition wherein the components so described are in a relationship permitting them to function in their intended manner. For instance, an RNA molecule comprising a “meristem transport sequence” (MTS) is operably linked or fused to a guide RNA if the MTS provide for delivery of the guide RNA to meristem cells.

As used herein, the terms “orthologous” or “orthologue” are used to describe genes or the RNAs or proteins encoded by those genes that are from different species but which have the same function (e.g., encode RNAs which exhibit the same meristem transport function). Orthologous genes will typically encode RNAs or proteins with some degree of sequence identity and can also exhibit conservation of sequence motifs, and/or conservation of structural features including RNA stem loop structures.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

As used herein, the phrase “substantially purified” defines an isolation of a molecule or compound in a form that is substantially free of contaminants normally associated with the molecule or compound in a native or natural environment and means having been increased in purity as a result of being separated from other components of the original composition. The phrase “substantially purified RNA molecule” is used herein to describe an RNA molecule which has been separated from other contaminant compounds including, but not limited to polypeptides, lipids, and carbohydrates. In certain embodiments, a substantially purified RNA is at least 90%, 95%, 97%, 98%, 99%, 99.5%, or 99.9% free of contaminating compounds by weight. A substantially purified RNA molecule can be combined with other compounds including buffers, RNase inhibitors, surfactants, and the like in a composition.

As used herein, the term “polynucleotide” refers to a nucleic acid molecule containing multiple nucleotides and encompasses both “oligonucleotides” (defined here as a polynucleotide molecule of between 2-25 nucleotides in length) and polynucleotides of 26 or more nucleotides. Polynucleotides are generally described as single- or double-stranded. Where a polynucleotide contains double-stranded regions formed by intra- or intermolecular hybridization, the length of each double-stranded region is conveniently described in terms of the number of base pairs. Aspects of this invention include the use of polynucleotides or compositions containing polynucleotides; embodiments include one or more oligonucleotides or polynucleotides or a mixture of both, including single- or double-stranded RNA or single- or double-stranded DNA or double-stranded DNA/RNA hybrids or chemically modified analogues or a mixture thereof. In various embodiments, a polynucleotide includes a combination of ribonucleotides and deoxyribonucleotides (e.g., synthetic polynucleotides consisting mainly of ribonucleotides but with one or more terminal deoxyribonucleotides or synthetic polynucleotides consisting mainly of deoxyribonucleotides but with one or more terminal dideoxyribonucleotides), or includes non-canonical nucleotides such as inosine, thiouridine, or pseudouridine. In embodiments, the polynucleotide includes chemically modified nucleotides (see, e.g., Verma and Eckstein Annu. Rev. Biochem. 1998, 67:99-134); for example, the naturally occurring phosphodiester backbone of an oligonucleotide or polynucleotide can be partially or completely modified with phosphorothioate, phosphorodithioate, or methylphosphonate internucleotide linkage modifications; modified nucleoside bases or modified sugars can be used in oligonucleotide or polynucleotide synthesis; and oligonucleotides or polynucleotides can be labelled with a fluorescent moiety (e.g., fluorescein or rhodamine or a fluorescence resonance energy transfer or FRET pair of chromophore labels) or other label (e.g., biotin or an isotope). Modified nucleic acids, particularly modified RNAs, are disclosed in U.S. Pat. No. 9,464,124, incorporated by reference in its entirety herein.

As used herein, the phrase “sequence identity” refers to the percent similarity of two polynucleotides or polypeptides. A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available at ncbi[dot]nlm[dot]nih[dot]gov/BLAST. See, e.g., Altschul et al. Mol. Biol. 1990, 215:403-410. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol., 70:173-187 (1997). Also, the GAP program using the Needleman and Wunsch alignment method can be utilized to align sequences. See Mol. Biol., 48:443-453 (1970).

As used herein, the terms “vascular system” or “vasculature” refer to the transport systems within the plant. This includes xylem, phloem, and cambium.

As used herein, the phrase “T-DNA” or “transfer DNA” refer to the DNA transferred from the tumor-inducing plasmid of species of bacteria such as, but not limited to, Agrobacterium tumefaciens and Agrobacterium rhizogenes (also known as Rhizobium rhizogenes), to the nuclear genome of a host plant.

As used herein, the phrase “T-DNA vector” refers to a transfer DNA vector system comprising as least a disarmed tumor inducing (Ti) plasmid of species of bacteria such as, but not limited to, Agrobacterium tumefaciens and Agrobacterium rhizogenes (also known as Rhizobium rhizogenes), containing a T-DNA and a vector backbone, and a helper plasmid containing vir virulence genes. A T-DNA vector system may be a binary vector system; a superbinary vector system wherein the Ti plasmid also comprises virulence genes (Komari et al. Plant Physiol 2007, 145(4): 1155-1160); or a ternary vector system wherein the system further comprises an accessory plasmid or virulence helper plasmid comprising an additional virulence gene cluster (Anand et al. Plant Mol Biol 2018, 97(1-2): 187-200).

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art.

Where a term is provided in the singular, the inventors also contemplate aspects of the invention described by the plural of that term.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### I. Method of Editing

### A. Editing of a Grafted Scion Mediated by Root Expression of Cas and Guide RNA

The present application provides methods of editing a genomic target in a plant scion comprising grafting the scion onto a rootstock comprising nucleic acid encoding a Cas nuclease and nucleic acid encoding a guide RNA for the Cas nuclease, wherein the nucleic acid encoding the guide RNA and the nucleic acid encoding the Cas nuclease are fused to a nucleic acid encoding a meristem transport segment (MTS). A rootstock provides nucleic acid encoding genome editing reagents, i.e., a Cas nuclease and a guide RNA for the Cas nuclease, to the plant vascular system. In some embodiments, RNA encoding the Cas9 nickase or Cas12 nuclease and the guide RNA are transported from the rootstock to the scion by the plant vascular system. In some embodiments, RNA encoding the Cas9 nickase or Cas12 nuclease and the guide RNA are transported from the rootstock to the scion through the phloem. In some embodiments, RNA encoding the Cas9 nickase or Cas12 nuclease is translated in the scion. In some embodiments, a meristem of the scion is edited.

Provided herein is a rootstock comprising nucleic acid encoding a Cas9 nickase or Cas12 nuclease and nucleic acid encoding a guide RNA for the Cas9 nickase or Cas12 nuclease, wherein the nucleic acid encoding the guide RNA and the nucleic acid encoding the Cas9 nickase or Cas12 nuclease are fused to nucleic acid encoding a meristem transport segment (MTS).

In some embodiments, the genome editing reagents are provided to the rootstock by infection with Agrobacterium rhizogenes (also known as Rhizobium rhizogenes), producing a rootstock with transgenic hairy roots. In some embodiments, the nucleic acid encoding the Cas9 nickase or Cas12 nuclease and the nucleic acid encoding the guide RNA are provided in the same vector. In some embodiments, the nucleic acid encoding the Cas9 nickase or Cas12 nuclease and the nucleic acid encoding the guide RNA are provided in different vectors. In some embodiments, the vector is a viral vector. In some embodiments, the vector is a T-DNA vector. In some embodiments, the vector is a viral vector or a T-DNA vector.

In some embodiments, the rootstock comprises nucleic acid encoding two or more, three or more, four or more, or five or more guide RNAs. In some embodiments, the nucleic acid encoding each of the two or more, three or more, four or more, or five or more guide RNAs is joined to an MTS.

In some embodiments, the rootstock further comprises nucleic acid encoding a detectable marker fused to a nucleic acid encoding an MTS.

In some embodiments, a scion is grafted onto the rootstock. The fusion of the meristem transport segment to nucleic acid encoding the genome editing reagents results in the genome editing reagents being transported to cells of the meristem of the scion through the plant vascular system, which connects the rootstock to the scion through the graft junction. Nucleic acid encoding the genome editing reagents are translated in the cytosol of cells of the scion meristem and imported into meristem nuclei, whereupon the genome of the meristem nuclei is edited. Edits made in the scion meristem are heritable as the meristem nuclei will form the reproductive tissues of the plant, including the gametes.

By this method, editing of the scion meristem can be accomplished without the introduction of a transgene to the genome of the scion. The scion and resulting progeny will be genetically edited without containing sequences encoding the Cas nuclease and the guide RNA in its genome. This will result in more consistent editing results, as there will be no element of randomness as to where a transgene will insert itself in the genome, or what levels of expression will result from each randomized insertion locus. The provided methods will also result in faster breeding and safety programs, as there is no possibility of off-target effects from insertion of a transgene into an inopportune location in the genome, and there is no need for additional breeding or selection to remove a transgene encoding genome editing reagents from the scion genome. Additionally, the provided line of rootstocks comprising genome editing reagents can be a modular tool for editing a number of existing elite plant lines. A single rootstock line can be used to transform many grafted scions, without the need to transform each scion. The provided methods will enlarge the capacity of a plant editing pipeline to make edits and observe the resulting phenotypes in genetic backgrounds of commercial relevance.

### B. Delivery of Guide RNA to Edit a Scion

The present application provides methods of editing a genomic target in a plant scion comprising grafting the scion onto a rootstock comprising nucleic acid encoding a Cas nuclease, wherein the nucleic acid encoding a Cas nuclease is fused to a nucleic acid encoding a meristem transport segment (MTS), and delivering to the scion a guide RNA for the Cas nuclease. In some embodiments, the Cas nuclease is delivered to the plant by infection with Agrobacterium rhizogenes (also known as Rhizobium rhizogenes), producing a plant with transgenic hairy roots. A rootstock provides nucleic acid encoding a Cas nuclease to the plant vascular system. In some embodiments, a scion is grafted onto the rootstock. The fusion of the meristem transport segment to nucleic acid encoding the Cas nuclease results in the nucleic acid encoding the Cas nuclease being transported to cells of the meristem of the scion through the plant vascular system, which connects the rootstock to the scion through the graft junction. Nucleic acid encoding the Cas nuclease is translated in the cytosol of cells of the scion meristem and imported into meristem nuclei.

In some embodiments, the method comprises delivering two or more, three or more, four or more, or five or more guide RNAs. In some embodiments, the two or more, three or more, four or more, or five or more guide RNAs are each joined to an MTS. In some embodiments, two or more guide RNAs are encoded by a single precursor RNA. In some embodiments, the two or more guide RNAs are each flanked by a direct repeat.

A guide RNA may be delivered to the meristem in a variety of ways. For example, in some embodiments, the guide RNA is delivered to the scion or directly to the meristem of the scion. In some embodiments, the guide RNA is delivered to the rootstock and transported into the scion. In some embodiments, the guide RNA is produced in vitro. In some embodiments, the guide RNA is methylated in vitro, such as by an RNA methylase, to promote mobility. In some embodiments, the guide RNA is fused to a meristem transport segment (MTS). Delivery of the guide RNA can occur through the following non-exhaustive list: through use of an RNA spray comprising the guide RNA and a simple surfactant (see, e.g., U.S. Pat. No. 9,121,022); by application of a composition comprising the guide RNA onto a leaf after rubbing the leaf with 200 grit sandpaper with a dowel; by spraying onto a leaf very fine glass beads coated with a composition comprising the guide RNA; by injection of a composition comprising the guide RNA into the stem; by infiltration of the leaf with a composition comprising the guide RNA; by direct uptake in the roots of a composition comprising the guide RNA; or by biolistic delivery to leaves or other tissue with circular DNA expressing the guide RNA. In some embodiments, delivery of the guide RNA comprises spraying a composition comprising the guide RNA onto the leaves, shoot, stem, and/or meristem. In some embodiments, the composition comprising the guide RNA comprises a surfactant. In some embodiments, the composition comprising the guide RNA comprises glass beads coated with the guide RNA. In some embodiments, delivery of the guide RNA comprises rubbing a composition comprising the guide RNA onto the leaves, shoot, stem, and/or meristem. In some embodiments, delivery of the guide RNA comprises injecting a composition comprising the guide RNA into the stem. In some embodiments, delivery of the guide RNA comprises leaf infiltration of a composition comprising the guide RNA into the leaf. In some embodiments, the leaf infiltration comprises forced infiltration using a needle-less syringe or vacuum pump. In some embodiments, the composition comprising the guide RNA comprises a nuclease inhibitor. In some embodiments, the nuclease inhibitor comprises an RNase inhibitor. In some embodiments, delivery of the guide comprises biolistic transformation of nucleic acid encoding the guide RNA into the leaf, shoot, shoot, stem, and/or meristem. In some embodiments, the biolistic transformation comprises transformation of circular DNA encoding the guide RNA.

In some embodiments, RNA encoding the Cas nuclease and/or the guide RNA is transported by plant vascular system. In some embodiments, RNA encoding the Cas nuclease and/or the guide RNA is transported to the scion through the xylem or the phloem. In some embodiments, RNA encoding the Cas nuclease and/or the guide RNA is transported to the meristem. In some embodiments, RNA encoding the Cas nuclease is translated in the meristem.

In some embodiments, the meristem is edited.

The guide RNA is transported to the meristem of the plant scion, or is provided to the meristem of the plant scion directly. The guide RNA is imported into the meristem nuclei. Upon import of both the Cas nuclease and the guide RNA for the Cas nuclease into the meristem nuclei, the genome of the meristem nuclei is edited. Edits made in the scion meristem are heritable as the meristem nuclei will form the reproductive tissues of the plant, including the gametes.

The provided methods allow for fast and modular editing of a multitude of plants, including elite lines, without the introduction of a transgene to the genome of the edited plant scion. Edits can be made in any plant that can be grafted onto a provided rootstock, including plant species that are intractable to transformation. Many scions from the same line can be grafted on rootstock plants providing the Cas nuclease, and different guide RNAs can be delivered to the different plant scions. Because there isn't a different transgene being inserted into a different location in each plant scion, this allows for direct comparison of the results of providing different guide RNAs, including but not limited to comparison of efficiency of method of delivery, editing efficiency of different guide RNAs, and phenotypic changes as a result of edits induced by different guide RNAs. The provided methods will enlarge the capacity of a plant editing pipeline to make edits and observe the resulting phenotypes in genetic backgrounds of commercial relevance.

Additionally, the provided methods allow for a reduced number of required transformation events. The rootstock providing the Cas nuclease can be used with a wide variety of delivered guide RNAs, increasing the modularity of the editing system.

### C. Uptake of Guide RNA by Roots for Editing a Plant

The present application provides methods of editing a genomic target in a plant meristem comprising providing a plant comprising nucleic acid encoding a Cas nuclease, wherein the nucleic acid encoding a Cas nuclease is fused to a nucleic acid encoding a meristem transport segment (MTS), and delivering to the root of the plant a guide RNA for the Cas nuclease. In some embodiments, the plant comprising the nucleic acid encoding a Cas nuclease is a rootstock. In some embodiments, a scion is grafted onto the rootstock. In some embodiments, the genomic editing reagents are provided to the plant by infection with Agrobacterium rhizogenes (also known as Rhizobium rhizogenes), producing a plant with transgenic hairy roots. In some embodiments, the Cas nuclease is delivered to the plant root by infection with Agrobacterium rhizogenes (also known as Rhizobium rhizogenes), producing a plant with transgenic hairy roots. The plant provides nucleic acid encoding a Cas nuclease to the plant vascular system. The fusion of the meristem transport segment to nucleic acid encoding the Cas nuclease results in the nucleic acid encoding the Cas nuclease being transported to cells of the meristem of the scion through the plant vascular system. In some embodiments, the nucleic acid encoding the Cas nuclease is transported from the rootstock to the scion through the graft junction. In some embodiments, RNA encoding the Cas nuclease and/or the guide RNA is transported by plant vascular system. In some embodiments, RNA encoding the Cas nuclease and/or the guide RNA is transported through the xylem or the phloem. In some embodiments, RNA encoding the Cas nuclease and/or the guide RNA is transported to the meristem, wherein the Cas nuclease and/or the guide RNA is translated in the meristem. Nucleic acid encoding the Cas nuclease is translated in the cytosol of cells of the scion meristem and imported into meristem nuclei.

In some embodiments, the guide RNA is delivered to the roots. In some embodiments, the guide RNA is delivered via direct uptake in the roots. In some embodiments, the guide RNA is delivered to the plant by infection with Agrobacterium rhizogenes (also known as Rhizobium rhizogenes), producing a plant with transgenic hairy roots. In some embodiments, the guide RNA is injected into the roots. In some embodiments, the guide RNA is produced in vitro. In some embodiments, the guide RNA is methylated in vitro, such as by an RNA methylase, to promote mobility. In some embodiments, the guide RNA is fused to a meristem transport segment (MTS). Delivery of the guide RNA can occur through the following non-exhaustive list: through use of an RNA spray comprising the guide RNA and a simple surfactant (see, e.g., U.S. Pat. No. 9,121,022); by injection of a composition comprising the guide RNA into the stem; by direct uptake in the roots of a composition comprising the guide RNA; or by biolistic transformation of roots or other tissue with circular DNA expressing the guide RNA. The guide RNA is transported to the meristem of the plant, and is imported into the meristem nuclei. Upon import of both the Cas nuclease and the guide RNA for the Cas nuclease into the meristem nuclei, the genome of the meristem nuclei is edited. Edits made in the scion meristem are heritable as the meristem nuclei will form the reproductive tissues of the plant, including the gametes.

The provided methods for editing a grafted scion allow for fast and modular editing of a multitude of plants, including elite lines, without the introduction of a transgene to the edited genome. Edits can be made in any plant that can be grafted onto a provided rootstock, including plant species that are intractable to transformation. Many scions from the same line can be grafted on the rootstock, allowing for direct comparison of the results of providing different guide RNAs, including but not limited to comparison of efficiency of method of delivery, editing efficiency of different guide RNAs, and phenotypic changes as a result of edits induced by different guide RNAs. The provided methods will enlarge the capacity of a plant editing pipeline to make edits and observe the resulting phenotypes in genetic backgrounds of commercial relevance.

The provided methods for editing a plant transformed with Agrobacterium rhizogenes allow for a fast and modular introduction of heritable edits. A strain of Agrobacterium is developed that comprises the Cas nuclease, and this strain can be used to infect and transform a variety of plants. This results in a variety of plants to which a guide RNA can be delivered to produce heritable edits in the plant meristem. This method does not require any additional generations between the transformation with Agrobacterium and the production of heritable edits, and is thus an improvement on current editing techniques.

### D. Grafting

In some embodiments, the method provided herein comprise editing a grafted scion. Grafting can be performed, for example, by inserting one or more cut scion stems into a cut of a rootstock stem, wherein the vascular tissue of the scion stem and the rootstock stem are substantially aligned. A stabilization device may be used.

A successful graft exhibits a continuous vascular system from rootstock to scion, including transmission through a graft junction. RNAs and/or endonucleases expressed in the rootstock, in some embodiments encoding genome editing reagents, enter the phloem and transit to the shoot apical meristem of the scion. The RNAs and/or endonucleases are imported into cells of the meristem and are processed into functional RNPs, which are able to modify the genome of the meristem of the plant scion. The present disclosure provides methods of editing the genome of a transgene-free plant scion, wherein the plant scion genome does not contain DNA encoding reagents for genomic modification. This technology enables one to introduce constructs encoding genome editing reagents into an easily transformable germplasm that can then be grafted to elite shoots as a rootstock, resulting in heritable genome edits in the scion.

A plant scion transformed through the present methods of genomic editing does not contain transgenes encoding the reagents for genomic modification. The plant scion must be able to be grafted onto a transformed rootstock, but it is not necessary that the plant scion itself be transformable. This widens the possibility of species that can be edited through the present disclosure. Additionally, many plants can be grafted onto the same variety of rootstock, thus speeding development of genomically edited scions.

### E. CRISPR-Cas Systems

CRISPR (Clustered Regularly Interspaced Short Palindromic Repeats)/Cas systems, or CRISPR systems, are adaptive defense systems originally discovered in bacteria and archaea. CRISPR systems use RNA-guided nucleases termed CRISPR-associated or “Cas” endonucleases (e.g., Cas9 or Cas12a (“Cpf1”)) to cleave foreign DNA. In a typical CRISPR/Cas system, a Cas endonuclease is directed to a target nucleotide sequence (e.g., a site in the genome that is to be sequence-edited) by sequence-specific, non-coding “guide RNAs” that target single- or double-stranded DNA sequences. In microbial hosts, CRISPR loci encode both Cas endonucleases and “CRISPR arrays” of the non-coding RNA elements that determine the specificity of the CRISPR-mediated nucleic acid cleavage.

The genomic DNA sequence targeted for editing or modification must generally be adjacent to a “protospacer adjacent motif” (“PAM”) that is specific for a given Cas endonuclease; however, PAM sequences are short and relatively non-specific, appearing throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). Some endonucleases, e.g., Cas9 endonucleases, are associated with G-rich PAM sites, e.g., 5′-NGG, and perform blunt-end cleaving of the target DNA at a location three nucleotides upstream from (5′ from) the PAM site. Cas12a (Cpf1) CRISPR systems cleave the target DNA adjacent to a short T-rich PAM sequence, e.g., 5′-TTN, in contrast to the G-rich PAM sequences identified for Cas9 systems. Examples of Cas12a PAM sequences include those for the naturally occurring Acidaminococcus sp. BV3L6 Cpf1 (AsCpf1) and Lachnospiraceae bacterium ND2006 Cpf1 (LbCpf1) TTTV, where V can be A, C, or G. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Various methods (including in silico and/or wet lab methods) for identification of the appropriate PAM sequence are known in the art and are routine, and any convenient method can be used. A PAM sequence can be identified using a PAM depletion assay. Cas12a cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. Cell 2015, 163:759-771.

### F. Nucleases

Two classes (1 and 2) of CRISPR systems have been identified across a wide range of bacterial hosts. The well characterized class 2 CRISPR systems use a single Cas endonuclease (rather than multiple Cas proteins). One class 2 CRISPR system includes a type II Cas endonuclease such as Cas9, a CRISPR RNA (“crRNA”), and a trans-activating crRNA (“tracrRNA”), see Guide RNA below. The Cas12a (“Cpf1”) CRISPR system includes the type V endonuclease Cas12a (also known as “Cpf1”). Cas12a nucleases are characterized as having only a RuvC nuclease domain, in contrast to Cas9 nucleases which have both RuvC and HNH nuclease domains. Cas12a nucleases are generally smaller proteins than Cas9 nucleases and can function with a smaller guide RNA (e.g., a crRNA having at least one spacer flanked by direct repeats), which are practical advantages in that the nuclease and guide RNAs are more economical to produce and potentially more easily delivered to a cell. Examples of Cas12a nucleases include AsCas12a or “AsCpf1” (from Acidaminococcus sp.) and LbCas12a or “LbCpf1” (from Lachnospiraceae bacteria). In contrast to Cas9 type CRISPR systems, Cas12a-associated (“Cpf1”-associated) CRISPR arrays have been reported to be processed into mature crRNAs without the requirement of a tracrRNA, i.e., the naturally occurring Cas12a (Cpf1) CRISPR system was reported to require only the Cas12a (Cpf1) nuclease and a Cas12a crRNA to cleave the target DNA sequence; see Zetsche et al. Cell 2015, 163:759-771; U.S. Pat. No. 9,790,490.

It is understood that for all systems, the use of a nuclease activity for cutting DNA followed by repair by the endogenous cell machinery is one solution to generate useful mutants. The nuclease activity can be eliminated or altered, as in dCas (“dead” Cas, i.e., Cas with no nuclease functionality) or nCas (“nickase” Cas, i.e., Cas that makes single-stranded breaks rather than double-stranded breaks), TALE (TAL-effector), or ZF (zinc finger) versions of the polypeptides. Inactivated nucleases can be useful for targeting the desired DNA sequence, while editing can be performed by nucleobase editors attached to the altered nucleases. Examples are included in WO2018176009 and U.S. Pat. No. 10,113,163, incorporated herein by reference.

Useful CRISPR-based RNA-guided nuclease systems have been described and are known from the literature, including but not limited to Cas9, Cas12a (Cpf1), Cas12e (CasX), Cas12d (CasY), C2c1, C2c2, C2c3 (see WO2018176009), Cas12h, Cas12i (see Yan et al. Science 2019, 363(6422): 88-91) and Cas12j (Pausch et al. Science 2020, 369(6501): 333-337). “Cas12” is used herein to refer to any Cas12 protein, including but not limited to Cas12a (Cpf1), Cas12e (CasX), Cas12d (CasY), C2c1, C2c2, C2c3 (see WO2018176009), Cas12h, Cas12i (see Yan et al. Science 2019, 363(6422): 88-91) and Cas12j (Pausch et al. Science 2020, 369(6501): 333-337. In some embodiments, the Cas nuclease is selected from the group consisting of Cas9, Cas12a (Cpf1), Cas12e (CasX), Cas12d (CasY), C2c1, C2c2, C2c3, Cas12h, Cas12i, and Cas12j. In some embodiments, the Cas nuclease is a Cas nickase. In some embodiments, the Cas nuclease is a Cas9 nickase or a Cas12 nuclease. In some embodiments, the Cas nickase is a Cas9 nickase or a Cas12 nickase. In some embodiments, the Cas nickase comprises mutation in one or more nuclease active sites. In some embodiments, the Cas nuclease is associated with a reverse transcriptase.

In a phenomenon termed “codon bias”, different organisms use specific codons more often than synonymous codons to encode for the same amino acid. Furthermore, efficiency of mRNA translation can be correlated with the use of the preferred codons over less frequently used codons. A nucleic acid can therefore be optimized for expression in a desired host by replacing codons less frequently used in that host with those more frequently used in the host. Codon bias varies across species, as well as across wider phylogenetic distance. Codon usage tables are known in the art (see, e.g., the “Codon Usage Database” at www[dot]kazusa[dot] or [dot]jp[forward slash]codon) and these tables can be adapted in a number of ways, as shown in Nakamura et al. (Nucl Acids Res 2000, 28:292). Computer algorithms may also be used for codon optimization of a particular sequence for expression in a desired host, such as Gene Forge (Aptagen; Jacobus, PA). For use in plants, see e.g. Campbell and Gowri (Plant Physiol 1990, 92:1-11) and Murray et al. (Nucl Acids Res 1989, 17:477-498.

A Cas nuclease is encoded by a nucleic acid. In one embodiment, the nucleic acid encoding the Cas nuclease is codon-optimized for use in a species of plant. In some embodiments, the nucleic acid encoding the Cas nuclease is codon-optimized for expression in dicots. In some embodiments, the nucleic acid encoding the Cas nuclease is codon-optimized for expression in soybean. In some embodiments, the nucleic acid encoding the Cas nuclease is codon-optimized for expression in monocots. In some embodiments, the nucleic acid encoding the Cas nuclease is codon-optimized for expression in corn. In some embodiments, the nucleic acid encoding the Cas nuclease is codon-optimized for expression in wheat. In some embodiments, the Cas nuclease is fused to a nuclear localization signal (NLS). CRISPR nuclease fusion proteins containing nuclear localization signals and codon-optimized for expression in maize are disclosed in U.S. patent application Ser. No. 15/120,110, published as U.S. Patent Application Publication 2017/0166912, national phase application claiming priority to PCT/US2015/018104 (published as WO/2015/131101 and claiming priority to U.S. Provisional Patent Application 61/945,700), incorporated herein by reference.

The nucleic acid encoding the Cas nuclease is fused to a nucleic acid encoding a meristem transport segment (MTS). In some embodiments, the nucleic acid encoding at least one guide RNA and the nucleic acid encoding the Cas nuclease are fused to one or more nucleic acids encoding a meristem transport segment. In some embodiments, RNA encoding the Cas nuclease and at least one guide RNA are transported from the rootstock to the scion by the plant vascular system. In some embodiments, RNA encoding the Cas nuclease and at least one guide RNA are transported from the rootstock to the scion through the xylem or the phloem. In some embodiments, RNA encoding the Cas nuclease and at least one guide RNA are transported from the rootstock to the scion through the plasmodesmata. In some embodiments, RNA encoding the Cas nuclease and at least one guide RNA are translated in the cytosol of a meristem cell. In some embodiments, translation of the RNA encoding the Cas nuclease and at least one guide RNA in the cytosol of a meristem cell results in editing of the genome of the meristem cell. In some embodiments, the meristem is on the plant scion.

In some embodiments, the nucleic acid encoding the Cas enzyme is operably linked to a promoter. For use in plants, useful promoters include constitutive, conditional, inducible, and temporally or spatially specific promoters (e.g., a tissue specific promoter, a developmentally regulated promoter, or a cell cycle regulated promoter). In some embodiments, the nucleic acid encoding the Cas enzyme is operably linked to a constitutive promoter. Examples of constitutive promoters include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, an opaline synthase (NOS) and octapine synthase (OCS) promoter from Agrobacterium tumefaciens, and a ubiquitin promoter. In some embodiments, the nucleic acid encoding the Cas enzyme is operably linked to an inducible promoter. An “inducible” promoter is a promoter that initiates transcription in response to an environmental stimulus such as heat, cold, drought, light, or other stimuli, such as wounding or chemical application. Examples of inducible promoters include, but are not limited to, those described in U.S. Pat. No. 6,294,714 (light inducible promoters), U.S. Pat. No. 6,140,078 (salt inducible promoters), U.S. Pat. No. 6,252,138 (pathogen inducible promoters), and U.S. Pat. No. 6,175,060 (phosphorus deficiency inducible promoters). In some embodiments, the nucleic acid encoding the Cas enzyme is operably linked to a promoter selected from the group consisting of promoters active in roots and promoter active in phloem companion cells. In some embodiments, the promoter active in roots is the promoter of a gene selected from the group consisting of Arabidopsis thaliana WRKY6 or orthologous genes thereof, chickpea WRKY31 or orthologous genes thereof, carrot MYB113 or orthologous genes thereof, corn GLU1 or orthologous genes thereof, strawberry RB7-type TIP-2 or orthologous genes thereof, and banana TIP2-2 or orthologous genes thereof. Additional suitable root promoters are provided in the RGPDB database (database of root-associated genes and promoters in maize, soybean, and sorghum) as described in Moisseyev et al. Database, 1-7 (2020). In some embodiments, the promoter active in phloem companion cells is selected from the group consisting of a promoter from a Flowering Locus T (FT) gene, a promoter from a Fabaceaen FOR1 gene (Noll et al. Plant Mol Biol 2007, 65(3): 285-294), a rice tungro bacilliform virus promoter (Yin et al. Plant J 1997, 12(5): 1179-1188), an RmlC-like cupins superfamily protein promoter (CN102002498B), a Commelina yellow mottle virus promoter (Medberry et al., Plant Cell 1992, 4:185-192), a wheat dwarf virus promoter (WO2003060135A2), a sucrose synthase promoter (Yang and Russell PNAS 1990, 87:4144-4148), a glutamine synthetase promoter (Edwards et al. PNAS 1990, 87:3459-3463), a phloem-specific isoform of plasmamembrane H+-ATPase promoter (DeWitt et al. Plant J. 1991, 1(1): 121-128), a JmjC domain-containing protein 18 (JMJ18) promoter (Yang et al., PLOS Genet 2012, 8(4): e1002664), and a phloem protein 2 (PP2) promoter (U.S. Pat. No. 5,495,007A).

The nucleic acid encoding the Cas nuclease and/or at least one guide RNA is intended to be transcribed in the rootstock. In some embodiments, the nucleic acid encoding the Cas nuclease is fused to a meristem transport segment (MTS). In some embodiments, the nucleic acid encoding the MTS is located 3′ of the nucleic acid encoding the Cas nuclease. In some embodiments, the nucleic acid encoding the MTS is located 5′ of the nucleic acid encoding the Cas nuclease. The nucleic acid encoding the Cas nuclease and/or guide RNA is intended to be transcribed in a cell of the rootstock, transported through the graft junction to the scion, and translated inside a scion meristem cell. In some embodiments, RNA encoding the Cas nuclease and/or the guide RNA is transported by plant vascular system. In some embodiments, RNA encoding the Cas nuclease and/or the guide RNA is transported from the rootstock to the scion by plant vascular system. In some embodiments, RNA encoding the Cas nuclease and/or the guide RNA is transported through the xylem or the phloem. In some embodiments, RNA encoding the Cas nuclease and/or the guide RNA is transported to the scion through the xylem or the phloem. In some embodiments, RNA encoding the Cas nuclease and/or the guide RNA is translated in the scion. In some embodiments, RNA encoding the Cas nuclease and/or the guide RNA is transported to the meristem, wherein the Cas nuclease and/or the guide RNA is translated in the meristem. As such, the nucleic acid encoding the Cas nuclease and/or the guide RNA is typically embedded within an mRNA component. A 5′ cap and polyA tail are also useful in stabilizing the RNA. A 5′ UTR has translation initiation sequences upstream of the Cas coding sequence. A 5′ UTR can also have small upstream open reading frames that affect translation (Jorgensen and Dorantes-Acosta, Front. Plant Sci 2012, 3:191). For example, an mRNA can comprise a 5′ UTR comprising a 7-methylguanosine cap at its 5′ terminus followed by an untranslated sequence and terminated by the translation initiation codon of the coding sequence (e.g., the Cas coding sequence).

The nucleic acid encoding the Cas nuclease can be optimized to increase nuclease activity and editing efficiency. In some embodiments, the nucleic acid encoding the Cas enzyme is operably linked to a nuclear localization signal (NLS), such as the NLS from SV40. Various NLSs, including those that bind to the major groove and/or the minor groove of an importin protein, are well known in the art, as in Kosugi et al. (J Biol Chem 2009, 284(1): 478-485). In some embodiments, the nucleic acid encoding the Cas nuclease is fused to a cell penetrating peptide (CPP), such as octa-arginine or nona-arginine or a homoarginine 12-mer oligopeptide, or a CPP disclosed in the database of cell-penetrating peptides CPPsite 2.0, publicly available at webs[dot]iiitd[dot]edu[dot]in/raghava/cppsite/(Kardani and Bolhassani J Mol Biol 2021, 433(11): 166703). In some embodiments, the nucleic acid encoding the Cas enzyme further comprises a terminator. By “terminator” is meant a DNA segment near the 3′ end of an expression cassette that acts as a signal to terminate transcription and directs polyadenylation of the resultant mRNA. Such a 3′ element is also sometimes referred to as a “3′-untranslated region” or “3′-UTR” or a “polyadenylation signal”. Non-limiting embodiments of terminators functional in eukaryotic cells include a U6 poly-T terminator, an SV40 terminator, an hGH terminator, a BGH terminator, an rbGlob terminator, a synthetic terminator functional in a eukaryotic cell, a 3′ element from an Agrobacterium sp. Gene, a 3′ element from a non-human animal gene, a 3′ element from a human gene, and a 3′ element from a plant gene, wherein the 3′ element terminate transcription of an RNA transcript located immediately 5′ to the 3′ element. Useful 3′ elements include: Agrobacterium tumefaciens nos 3′, tml 3′, tmr 3′, tins 3′, ocs 3′, and tr7 3′ elements disclosed in U.S. Pat. No. 6,090,627, incorporated herein by reference; 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in U.S. Patent Application Publication 2002/0192813 A1, incorporated herein by reference; in some embodiments, the terminator is selected from the group consisting of CaMV 35S terminator, Atug7 terminator, NOS terminator, Act2 terminator, MAS terminator, tomato ATPase terminator, rbcSC3 terminator, potato H4 terminator, rbcSE9 terminator, GILT terminator, ALB terminator, API terminator, HSP terminator, and OCS terminator, as referenced in Hassan et al. (Trends Plant Sci 2021, 26:1133-1152). In some embodiments, the nucleic acid encoding the Cas enzyme further comprises one or more introns. In some embodiments, the nucleic acid encoding the Cas enzyme further comprises one or more transcriptional enhancers. In some embodiments, the one or more transcriptional enhancers comprise one or more bacterial octopine synthase (OCS) enhancers (U.S. Pat. No. 11,198,885). In one embodiment, the nucleic acid encoding the Cas enzyme further comprises a triple OCS enhancer (U.S. Pat. No. 11,198,885). In some embodiments, the nucleic acid encoding the Cas enzyme further comprises a 5′ UTR comprising a translational enhancer. In some embodiments, the nucleic acid encoding the Cas enzyme further comprises a Kozak sequence endogenous to the scion species at the translation start codon. In some embodiments, the nucleic acid encoding the Cas enzyme further comprises nuclear localization signals flanking the coding sequence of the Cas enzyme.

### G. Guide RNAs

CRISPR-based RNA-guided nuclease systems typically require an effector polypeptide and one or more guide RNAs (gRNAs). The guide RNAs are generally made up of an effector-binding region and a target DNA recognition region, and in some embodiments include tracrRNAs. A “trans-activating crRNA” or “tracrRNA” is a trans-encoded small RNA that is partially homologous to repeats within a CRISPR array. At least in the case of Cas9 type CRISPR systems, both a tracrRNA and a crRNA are required for the CRISPR array to be processed and for the nuclease to cleave the target DNA sequence. In contrast, Cas12a type CRISPR systems have been reported to function without a traceRNA, with the Cas12a CRISPR arrays processed into mature crRNAs without the requirement of a tracrRNA; see Zetsche et al. Cell 2015, 163:759-771 and U.S. Pat. No. 9,790,490. The Cas9 crRNA contains a “spacer sequence”, typically an RNA sequence of about 20 nucleotides (in various embodiments this is 20, 21, 22, 23, 24, 25, or up to about 30 contiguous nucleotides in length) that corresponds to (e.g., is identical or nearly identical to, or alternatively is complementary or nearly complementary to) a target DNA sequence of about equivalent length. The Cas9 crRNA also contains a region that binds to the Cas9 tracrRNA to form a partially double-stranded structure which is cleaved by RNase III, resulting in a crRNA: tracrRNA hybrid or duplex. The crRNA: tracrRNA hybrid then directs the Cas9 endonuclease to recognize and cleave the target DNA sequence; in some examples, a tracrRNA and crRNA (e.g., a crRNA including a spacer sequence) can be included in a chimeric nucleic acid referred to as a “single guide RNA” (sgRNA).

As used herein “guide RNA” or “gRNA” refers to a nucleic acid that comprises or includes a nucleotide sequence (sometimes referred to a “spacer sequence”) that corresponds to (e.g., is identical or nearly identical to, or alternatively is complementary or nearly complementary to) a target DNA sequence (e.g., a contiguous nucleotide sequence that is to be modified) in a genome; the guide RNA functions in part to direct the CRISPR nuclease to a specific location on the genome. In embodiments, a gRNA is a CRISPR RNA (“crRNA”), such as the engineered Cas12a crRNAs described in this disclosure. For nucleases (such as a Cas9 nuclease) that require a combination of a trans-activating crRNA (“tracrRNA”) and a crRNA for the nuclease to cleave the target nucleotide sequence, the gRNA can be a tracrRNA: crRNA hybrid or duplex, or can be provided as a single guide RNA (sgRNA). At least 16 or 17 nucleotides of gRNA sequence corresponding to a target DNA sequence are required by Cas9 for DNA cleavage to occur; for Cas12a (Cpf1) at least 16 nucleotides of gRNA sequence corresponding to a target DNA sequence are needed to achieve detectable DNA cleavage and at least 18 nucleotides of gRNA sequence corresponding to a target DNA sequence were reported necessary for efficient DNA cleavage in vitro; see Zetsche et al. Cell 2015, 163:759-771. Cas12a (Cpf1) endonuclease and corresponding guide RNAs and PAM sites are disclosed in U.S. Pat. No. 9,790,490, which is incorporated herein by reference in its entirety and particularly for its disclosure of DNA encoding Cas12a (Cpf1) endonucleases and guide RNAs and PAM sites. In practice, guide RNA sequences are generally designed to contain a spacer sequence of between 17-24 contiguous nucleotides (frequently 19, 20, or 21 nucleotides) with exact complementarity (e.g., perfect base-pairing) to the targeted gene or nucleic acid sequence; guide RNAs having spacers with less than 100% complementarity to the target sequence can be used (e.g., a gRNA with a spacer having a length of 20 nucleotides and between 1-4 mismatches to the target sequence), but this can increase the potential for off-target effects. The design of effective guide RNAs for use in plant genome editing is disclosed in U.S. Patent Application Publication 2015/0082478 A1, the entire specification of which is incorporated herein by reference. Chemically modified sgRNAs have been demonstrated to be effective in Cas9 genome editing; see, for example, Hendel et al. Nature Biotechnol., 2015, 33:985-991.

Guide RNA(s) can be part of the same RNA (mRNA) capable of expressing the Cas nuclease. In one embodiment, one or more guide RNAs are flanked by direct repeats (DR) of the CRISPR array from which the Cas effector polypeptide was first isolated. In some embodiments, the two or more guide RNAs are each flanked by a direct repeat. For example, a translated and expressed active Cas12a nuclease can process the DR-flanked spacers of the mRNA to make guide RNAs. In certain embodiments, a translated and expressed active Cas12a nuclease can process Cas12a DR-flanked spacers of the mRNA to make guide RNAs. In certain embodiments, a translated and expressed active Cas12e nuclease can process Cas12e DR-flanked spacers of the mRNA to make guide RNAs. In certain embodiments, a translated and expressed active Cas12i nuclease can process Cas12i DR-flanked spacers of the mRNA to make guide RNAs. In certain embodiments, a translated and expressed active Cas12j nuclease can process Cas12j DR-flanked spacers of the mRNA to make guide RNAs. In alternative embodiments, a guide RNA suitable for matching an expressed effector polypeptide is flanked by processing elements, so that functional guide RNAs are excised inside the cells. Exemplary processing elements include hammerhead ribozymes, Csy4, and tRNAs (see Mikami et al. Plant Cell Physiol. 2017, 58(11): 1857-1867; and U.S. Pat. No. 10,308,947). Ribozymes can autocatalytically cleave the RNA to release the guide RNA from a polycistronic transcript and/or remove additional 5′ or 3′ sequence around the guide RNA. tRNAs are processed by elements of the cell's endogenous tRNA system, such as RNase P, RNase Z, and RNase E, and tRNA sequences or pre-tRNA sequences can also be used to release a guide RNA flanked by processing elements from a polycistronic transcript and/or remove additional 5′ or 3′ sequence around the guide RNA. In some embodiments, the nucleic acid encoding the guide RNA and the MTS is located between two ribozyme sequences. In some embodiments, each of the ribozyme sequences is independently selected from the group consisting of a hammerhead ribozyme sequence, a HDV ribozyme sequence, a Csy4 sequence, and a tRNA processing enzyme sequence. In some embodiments, the nucleic acid encoding the guide RNA and the MTS further comprises a hammerhead ribozyme sequence 5′ to the nucleic acid encoding the guide RNA and the MTS, and a HDV ribozyme 3′ to the nucleic acid encoding the guide RNA and the MTS. In some embodiments, a guide RNA is encoded by a nucleic acid. In some embodiments, the guide RNA is fused to a meristem transport segment (MTS). In some embodiments, the nucleic acid encoding the MTS is located 3′ of the nucleic acid encoding the Cas9 nickase or Cas12 nuclease and/or 3′ of the nucleic acid encoding the guide RNA. In some embodiments, the nucleic acid encoding the MTS is located 5′ of the nucleic acid encoding the Cas9 nickase or Cas12 nuclease and/or 5′ of the nucleic acid encoding the guide RNA.

In some embodiments, the guide RNA comprises at its 3′ end a priming site and an edit to be incorporated into the genomic target. In some embodiments, the nucleic acid encoding the guide RNA and the MTS further comprises a terminator. In some embodiments, the terminator is a U6 terminator.

In some embodiments, the guide RNA comprises a 5-methylcytosine group.

In some embodiments, the present invention comprises a guide RNA or guide RNA(s) which have chemical modifications. Chemical modifications are made to RNA molecules which then alter at least one of the four canonical ribonucleotides: A, U, C, and G. These modifications can be natural or unnatural and refer to a chemical moiety or portions of a chemical moiety which are not found in the unmodified canonical ribonucleotides. Alternative bases can include but are not limited to 2-thiouridine, 4-thiorudine, 2-aminoadenosine, 7-deazaguanosine, inosine, 5-methylcytidine, 5-aminoallyluridine, and 5-methyluridine. Either independently or additionally, a guide RNA which comprises any backbone or inter-nucleotide linkage other than a natural phosphodiester linkage is a chemically modified guide RNA. Alternative phosphodiester linkages can include but are not limited to an alkylphosphonate, a phosphonocaboxylate, a phosphonoacetate, a boranophosphonate, a phosphorothioate, a phosphonothioacetate, and a phoshporodithioate linkage. Either independently or additionally, a guide RNA which comprises labeled isotopes, such as one or more of ¹⁵N, ¹³C, ¹⁴C, Deuterium, or ³²P, or other atoms used as tracers, is a modified guide RNA. Either independently or additionally, a guide RNA which comprises modifications made to the sugar group is a chemically modified RNA. Sugar group modifications can include but are not limited to 2′-O-methyl, 2′-deoxy, 2′-methoxyethyl, 2′fluoro, 2′-amino, a sugar in L form, and 4′-thioribosyl.

In certain embodiments, chemical modifications protect the guide RNA from nucleases. In certain embodiments, this modification aids in the stability of the RNA molecules, where the half-life of the chemically modified RNA molecule is altered from the unmodified form. In certain embodiments, the chemically modified guide RNA maintains its functionality, which includes guide RNA binding to a Cas protein. In some embodiments, this maintained functionality of the gRNA includes binding a target polynucleotide. In some embodiments, the maintained functionality of the guide RNA includes binding both a Cas protein and a polynucleotide in complex. In some embodiments, the chemical modifications on the guide RNA are used to distinguish the sequences from the nascent sequences present in the experimental plant. In certain embodiments, the chemical modifications alter the prevalence of off-target cleavage events, where “off-target” is defined as a site in the target genome that is different from the site at which the guide RNA was designed to induce a cleavage event.

Chemical modifications to guide RNAs are known in the art, for example in U.S. Pat. No. 10,337,001, and Ryan et al. 2018, Nucleic Acids Res. 46(20): 792-803.

In some embodiments the guide RNA further comprises (a) one or more modified nucleotides within five nucleotides from the 5′ end of the guide RNA; or (b) one or more modified nucleotides within five nucleotides from the 3′ end of the guide RNA; or (c) both (a) and (b); wherein the one or more modified nucleotides has a modification to a phosphodiester linkage, a sugar, or both a phosphodiester linkage and a sugar. In some embodiments, each of the one or more modified nucleotides is independently selected from the group consisting of a 2′-O-methyl nucleotide, a 2′-O-methyl-3′-phosphorothioate nucleotide, a 2′-O-methyl-3′-phosphonoacetate nucleotide, and a 2′-O-methyl-3′-phosphonothioacetate nucleotide. In some embodiments, the one or more modified nucleotide comprises a modified internucleotide linkage or a modified terminal phosphate group selected from the group consisting of an alkylphosphonate, a phosphonocarboxylate, a phosphonoacetate, a boranophosphonate, a phosphorothioate, a phosphonothioacetate, and a phosphorodithioate group.

In some embodiments, the nucleic acid encoding the guide RNA is operably linked to a promoter. In some embodiments, the promoter is an RNA polymerase II promoter or an RNA polymerase III promoter. In some embodiments, the RNA polymerase II promoter or RNA polymerase III promoter is endogenous to the species of the rootstock.

In some embodiments, a single guide RNA is provided to the plant. In other embodiments, multiple guide RNAs are provided to the plant. In some embodiments, the multiple guide RNAs are provided in a CRISPR array. In some embodiments, the two or more guide RNAs are encoded by a single precursor RNA. For the purposes of gene editing, CRISPR arrays can be designed to contain one or multiple guide RNAs designed to target a DNA sequence for editing, where the guide RNA includes at least one spacer sequence that corresponds to a specific locus of about equivalent length in the target DNA; see, for example, Cong et al. Science, 2013, 339:819-823; Ran et al. Nature Protocols, 2013, 8:2281-2308. In some embodiments, the CRISPR array comprises more than one spacer sequence. In some embodiments, the CRISPR array comprises more than one distinct spacer sequences. In some embodiments, the CRISPR array comprises more than one distinct spacer sequences designed to target the same genomic locus. In some embodiments, the CRISPR array comprises more than one distinct spacer sequences designed to target more than one distinct genomic loci. In some embodiments, the multiple guide RNAs are provided in a polycistronic system, wherein the multiple guide RNAs are operably linked to a single promoter. In other embodiments, the multiple guide RNAs are operable linked to multiple promoters. In some embodiments, the multiple guide RNAs are operably linked to multiple copies of the same promoter. In some embodiments, the multiple guide RNAs are operably linked to different promoters. In some embodiments, the multiple guide RNAs target the same genomic locus. In other embodiments, the multiple guide RNAs target multiple genomic loci. In some embodiments, the multiple guide RNAs are provided in a CRISPR array, wherein the CRISPR array is operably linked to a single MTS. In some embodiments, the method comprises applying two or more, three or more, four or more, or five or more guide RNAs. In some embodiments, the two or more, three or more, four or more, or five or more guide RNAs are each joined to an MTS. In some embodiments, the multiple guide RNAs are provided in a polycistronic system, wherein the multiple guide RNAs are operably linked to a single meristem transport segment (MTS). In other embodiments, the multiple guide RNAs are operable linked to multiple MTSs. In some embodiments, the multiple guide RNAs are operably linked to multiple copies of the same MTS. In some embodiments, the multiple guide RNAs are operably linked to different MTSs.

In some embodiments, delivery of the guide RNA comprises spraying a composition comprising the guide RNA onto the leaves, shoot, stem, and/or meristem. In som embodiments, the composition comprising the guide RNA comprises a surfactant. In some embodiments, the composition comprising the guide RNA comprises glass beads coated with the guide RNA.

In some embodiments, delivery of the guide RNA comprises rubbing a composition comprising the guide RNA onto the leaves, shoot, stem, and/or meristem.

In some embodiments, delivery of the guide RNA comprises injecting a composition comprising the guide RNA into the stem.

In some embodiments, delivery of the guide RNA comprises leaf infiltration of a composition comprising the guide RNA into the leaf. In some embodiments, the leaf infiltration comprises forced infiltration using a needle-less syringe or vacuum pump.

In some embodiments, the guide RNA is delivered to the plant root by incubating the root with a composition comprising the guide RNA.

In some embodiments, the guide RNA is delivered to the plant root by an Agrobacterium rhizogenes transformation. In some embodiments, the Agrobacterium rhizogenes transformation produces transgenic hairy roots.

In some embodiments, the guide RNA is delivered to the plant root by injecting a composition comprising the guide RNA into the root.

In some embodiments, the composition comprising the guide RNA comprises a nuclease inhibitor, optionally, wherein the nuclease inhibitor is an RNase inhibitor.

In some embodiments, the composition comprising the guide RNA comprises a nuclease inhibitor. In some embodiments, the nuclease inhibitor comprises an RNase inhibitor.

In some embodiments, application comprises biolistic transformation of nucleic acid encoding the guide RNA into the leaf, shoot, shoot, stem, and/or meristem. In some embodiments, the biolistic transformation comprises transformation of circular DNA encoding the guide RNA.

### H. Prime Editing

Desired DNA sequence modifications can be accomplished through the use of PRIME editing (Anzalone et al. Nature 2019, 576(7785): 149-157). In some embodiments, prime editing uses (i) a Cas nickase, in some embodiments a Cas9 nickase, in other embodiments a Cas12 nickase, fused to a reverse transcriptase (nCas-RT), in some embodiments a M-MLV reverse transcriptase, and (ii) a prime editing Cas guide RNA (pegRNA) that both specifies the genome target site and has an extension that encodes the target edit within a template for the reverse transcriptase. The binding of the pegRNA directs the Cas nickase to create a single-stranded break in the DNA at the nicking site. The extension of the pegRNA binds to the nicked DNA that has an exposed 3′-hydroxyl group, priming the reverse transcriptase to produce a DNA strand that is complementary to the extension of the pegRNA. This DNA strand will include the complement to any desired edits present in the provided pegRNA extension. Mismatch repair by the cell will then resolve the mismatch between the unedited parent strand and the edited product of the reverse transcriptase, thus introducing the desired edits into the genome. Prime editing systems may also include elements to inhibit mismatch repair, or to nick the unedited parent strand to increase editing efficiency. A mobility element can be fused to the pegRNA so as not to interfere with priming of the reverse transcriptase.

In some embodiments, prime editing can also be accomplished with Cas nucleases in place of Cas nickases (Adikusuma et al. Nucleic Acids Res. 2021, 49(18): 10785-10795). In some embodiments, prime editing uses (i) a Cas nuclease, in some embodiments a Cas9 nuclease, in other embodiments a Cas12 nuclease, fused to a reverse transcriptase (Cas-RT), in some embodiments a M-MLV reverse transcriptase, and (ii) a prime editing Cas guide RNA (pegRNA) that both specifies the genome target site and has an extension that encodes the target edit within a template for the reverse transcriptase. In some embodiments, the binding of the pegRNA directs the Cas nuclease to create a double-stranded break in the DNA at the target site. The extension of the pegRNA binds to the cut DNA that has an exposed 3′-hydroxyl group, priming the reverse transcriptase to produce a DNA strand that is complementary to the extension of the pegRNA. This DNA strand will include the complement to any desired edits present in the provided pegRNA extension. Mismatch repair by the cell will then resolve the mismatch between the unedited parent strand and the edited product of the reverse transcriptase, thus introducing the desired edits into the genome. Prime editing systems may also include elements to inhibit mismatch repair, or to nick the unedited parent strand to increase editing efficiency. A mobility element can be fused to the pegRNA so as not to interfere with priming of the reverse transcriptase.

Prime editing makes precise DNA sequence modifications rather than random insertions, deletions, and substitutions (Indels), thus increasing the probability of obtaining the desired effect. Prime editing may be used to introduce any single base pair substitution as well as small deletion or insertions. Deletions of up to 80 base pairs have been produced using prime editing with a single pegRNA in human cells, and insertions of up to 40 base pairs (Anzalone et al. Nature 2019, 576:149-157). Dual pegRNA systems are also known in the art (Choi et al. Nat Biotechnol 2021, 40(2): 218-226; Lin et al. Nature Biotechnology 2021, 39(8): 923-927) and can be used to generate precise large deletions, or to improve editing efficiency for small insertions, deletions, or substitutions. Additionally, dual pegRNA systems where the extension of the pegRNAs are not complementary to the endogenous locus, but are complementary to one another, can be used to replace endogenous sequence and/or mediate larger insertions (Anzalone et al. Nat Biotechnol 2022, 40(5): 731-740).

In some embodiments, the Cas nuclease is associated with a reverse transcriptase. In some embodiments, the Cas nuclease is fused to the reverse transcriptase. In some embodiments, the guide RNA comprises at its 3′ end a priming site and an edit to be incorporated into the genomic target. In some embodiments, the Cas nuclease is a Cas nickase. In some embodiments, the Cas nickase is a Cas9 nickase or a Cas12 nickase. In some embodiments, the Cas nickase comprises mutation in one or more nuclease active sites.

### I. Delivery to the Meristem

In some embodiments, the methods provided herein involve transport of one or more components of a gene editing systems (e.g. a Cas nuclease and a guide RNA) to the meristem. Meristem transport segments travel through the plant, typically but not limited to via the phloem, and are taken up into meristematic tissues. The examples below are sequences from individual species, which sometimes work across species. For example, Arabidopsis FT-based vectors work in Nicotiana benthamiana and Arabidopsis. Vectors can also be designed based on alternative sequences, which can be based either on the species subject to genomic editing or based on a different species, sometimes a related species, sometimes a closely related species.

While the transport segment is based on a plant-transported RNA, its actual sequence may be a fragment determined by characterizing a deletion series to make a smaller sequence retaining the desired transport (phloem mobility and/or meristem cell translocation) capabilities. The initiator methionine codon or translation initiation codon of the base sequence may also be mutated in some cases.

The Flowering Locus T (FT) mRNA is useful as a meristem transport segment. SEQ ID NO: 2 shows the DNA sequence that encodes the Arabidopsis FT RNA, and SEQ ID NO: 1 is a fraction of SEQ ID NO: 2 that encodes the RNA that functions as a transport segment. Alternative useful FTs may be ZCN8 (encoded by SEQ ID NO: 3), which may work across related monocot species. Alternative useful FTs may be GmFT2a (Sun et al. PLOS One. 2011, 6(12): e29238. doi: 10.1371/journal.pone.0029238; Jiang et al. BMC Genomics. 2019 20(1): 230. doi: 10.1186/s12864-019-5577-5; Kong et al. Plant Physiol. 2010 November, 154(3): 1220-31. doi: 10.1104/pp. 110.160796; Takeshima et al. J Exp Bot. 2019 Aug. 7, 70(15): 3941-3953. doi: 10.1093/jxb/erz199), which may work across related dicot species. FT RNA molecules that can be used include: (i) RNAs set forth in SEQ ID NO: 5, 6, 7, 8, 9, 10, 11, 12, 13, 14, 15, 16, 17, 18, 19, 20, 21, 22, 23, or 24, a meristem transport-competent (MTC) ortholog thereof, a MTC variant thereof, and/or a MTC fragment thereof; (ii) allelic variants of SEQ ID NO: 5, 6, 7, 8, 9, 10, 11, 12, 13, 14, 15, 16, 17, 18, 19, 20, 21, 22, 23, or 24, a meristem transport-competent (MTC) ortholog thereof, a MTC variant thereof, and/or a MTC fragment thereof; (iii) FT RNAs from various plants set forth in US20190300890, which is incorporated herein by reference in its entirety, allelic variants thereof, and meristem transport-competent (MTC) orthologs thereof, MTC variants thereof, and/or MTC fragments thereof; and tRNA-like sequences (TLSs) (Zhang et al. Plant Cell 2016, 28:1237-1249), variants thereof, and fragments thereof. FT RNA molecules that can be used include RNAs having at least 85%, 90%, 95%, 98%, or 99% sequence identity to SEQ ID NO: 5, 6, 7, 8, 9, 10, 11, 12, 13, 14, 15, 16, 17, 18, 19, 20, 21, 22, 23, 24, or a meristem transport-competent (MTC) fragment thereof.

More generally, viral and cellular-derived RNA molecules that are useful as part of a transport segment include the mRNAs of FT, GAI, CmNACP, tomato LeT6, a KNOX gene, BEL5, or tRNA-like sequences (Ruiz-Medrano et al. Development 1999, 126:4405-4419; Kim et al. Science 2001, 293:287-289; Haywood et al. Plant J. 2005, 42:49-68; and Li et al. Sci. Rep. 2011, 1:73; Cho et al. J. Exp. Bot 2015, 66:6835-6847; Zhang et al. Plant Cell 2016, 28:1237-1249; and WO2017178633). GAI RNAs that can be used include: (i) RNAs set forth in SEQ ID NO: 26, a meristem transport-competent (MTC) ortholog thereof, a MTC variant thereof, and/or a MTC fragment thereof; (ii) allelic variants of SEQ ID NO: 26, a meristem transport-competent (MTC) ortholog thereof, a MTC variant thereof, and/or a MTC fragment thereof; and (iii) RNAs having at least 85%, 90%, 95%, 98%, or 99% sequence identity to SEQ ID NO: 26, or a meristem transport-competent (MTC) fragment thereof. CmNACP RNAs that can be used include: (i) RNAs set forth in SEQ ID NO: 25, a meristem transport-competent (MTC) ortholog thereof, a MTC variant thereof, and/or a MTC fragment thereof; (ii) allelic variants of SEQ ID NO: 25, a MTC variant thereof, and/or a MTC fragment thereof; and (iii) RNAs having at least 85%, 90%, 95%, 98%, or 99% sequence identity to SEQ ID NO: 25, or a meristem transport-competent (MTC) fragment thereof. LeT6 RNAs that can be used include: (i) RNAs set forth in SEQ ID NO: 27, a meristem transport-competent (MTC) ortholog thereof, a MTC variant thereof, and/or a MTC fragment thereof; (ii) allelic variants of SEQ ID NO: 27, a MTC variant thereof, and/or a MTC fragment thereof; and (iii) RNAs having at least 85%, 90%, 95%, 98%, or 99% sequence identity to SEQ ID NO: 27, or a meristem transport-competent (MTC) fragment thereof. BEL5 RNAs that can be used include: (i) RNAs set forth in SEQ ID NO: 28, a meristem transport-competent (MTC) ortholog thereof, a MTC variant thereof, and/or a MTC fragment thereof; (ii) allelic variants of SEQ ID NO: 28, a MTC variant thereof, and/or a MTC fragment thereof; and (iii) RNAs having at least 85%, 90%, 95%, 98%, or 99% sequence identity to SEQ ID NO: 28, or a meristem transport-competent (MTC) fragment thereof. Examples of tRNA-like RNAs that can be used include: (i) RNAs set forth in SEQ ID NO: 29, 30, a meristem transport-competent (MTC) ortholog thereof, a MTC variant thereof, and/or a MTC fragment thereof; (ii) allelic variants of SEQ ID NO: 29, 30, a MTC variant thereof, and/or a MTC fragment thereof; and (iii) RNAs having at least 85%, 90%, 95%, 98%, or 99% sequence identity to SEQ ID NO: 29, 30, or a meristem transport-competent (MTC) fragment thereof. In certain embodiments, a TLS sequence, SEQ ID NO: 29 or 30, a meristem transport-competent (MTC) ortholog thereof, a MTC variant thereof, and/or an MTC fragment thereof can comprise an RNA hairpin comprising a first stem of 8 to 12 nucleotides, at least one variable bulge, a second stem of 4 to 7 nucleotides, and a variable loop. TLS sequences suitable for RNA transport and the structural features of such RNAs are set forth in Zhang et al. Plant Cell. 2016 Jun. 28(6): 1237, doi.org/10.1105/tpc.15.01056.

Further description of biological sequences provided in the sequence listing is set forth in Table 1. RNA molecules set forth in SEQ ID NO: 9-30 are respectively encoded by the DNA molecules set forth in SEQ ID NO: 31-52.

TABLE 1
Description of biological sequences.

| SEQ ID | | | Sequence (Polynucleotide: 5′ to 3′; |
|---|---|---|---|
| NO: | TYPE | Comments | Polypeptide: N to C) |
| 1 | DNA | Fragment of | ATGTCTATAAATATAAGAGACCCTCTTATAGTAAG |
| | | NM_001334207.1 | CAGAGTTGTTGGAGACGTTCTTGATCCGTTTAATA |
| | | Arabidopsis | GATCAATCACTCTAAAGGTTACTTATGGCCAAA |
| | | thaliana PEBP | |
| | | (phosphatidylet | |
| | | hanolamine- | |
| | | binding protein) | |
| | | family protein | |
| | | (FT), mRNA | |
| 2 | DNA | NM_001334207.1 | AGTTAATGCAAATCCGAAACAGTATAAATATGTG |
| | | Arabidopsis | TAGAGGGTTCATGCCTATGATACAAATTAAAGAA |
| | | thaliana PEBP | GCAGAAACAAAAACAAGTAAAACAGAAACAATC |
| | | (phosphatidylet | AACACAGAGAAACCACCTGTTTGTTCAAGATCAA |
| | | hanolamine- | AGATGTCTATAAATATAAGAGACCCTCTTATAGTA |
| | | binding protein) | AGCAGAGTTGTTGGAGACGTTCTTGATCCGTTTAA |
| | | family protein | TAGATCAATCACTCTAAAGGTTACTTATGGCCAAA |
| | | (FT), mRNA | GAGAGGTGACTAATGGCTTGGATCTAAGGCCTTCT |
| | | | CAGGTTCAAAACAAGCCAAGAGTTGAGATTGGTG |
| | | | GAGAAGACCTCAGGAACTTCTATACTTTGGTTATG |
| | | | GTGGATCCAGATGTTCCAAGTCCTAGCAACCCTCA |
| | | | CCTCCGAGAATATCTCCATTGGTTGGTGACTGATA |
| | | | TCCCTGCTACAACTGGAACAACCTTTGGCAATGAG |
| | | | ATTGTGTGTTACGAAAATCCAAGTCCCACTGCAGG |
| | | | AATTCATCGTGTCGTGTTTATATTGTTTCGACAGC |
| | | | TTGGCAGGCAAACAGTGTATGCACCAGGGTGGCG |
| | | | CCAGAACTTCAACACTCGCGAGTTTGCTGAGATCT |
| | | | ACAATCTCGGCCTTCCCGTGGCCGCAGTTTTCTAC |
| | | | AATTGTCAGAGGGAGAGTGGCTGCGGAGGAAGAA |
| | | | GACTTTAGATGGCTTCTTCCTTTATAACCAATTGA |
| | | | TATTGCATACTCTGATGAGATTTATGCATCTATAG |
| | | | TATTTTAATTTAATAACCATTTTATGATACGAGTA |
| | | | ACGAACGGTGATGATGCCTATAGTAGTTCAATAT |
| | | | ATAAGTGTGTAATAAAAATGAGAGGGGGAGGAA |
| | | | AATGAGAGTGTTTTACTTATATAGTGTGTGATGCG |
| | | | ATAATTATATTAATCTACATGAAATGAAGTGTTAT |
| | | | ATTTATACTTTACGTGTATTCATTTCTTTTCGATGC |
| | | | AAAAATCAGGCAGTGGGAAGAATCTGCTGTTTTA |
| | | | CTTTTG |
| 3 | DNA | EU241924.1 | TTGAGAGTTCTAATAAGAGCAACGGCCAATACCA |
| | | Zea mays | TTAGCGAGTTATTTTTCTGCAATATATGTCAGCAA |
| | | ZCN8 (ZCN8) | CCGATCATTTGGTTATGGCTCGTGTCATACAGGAT |
| | | mRNA, | GTATTGGATCCCTTTACACCAACCATTCCACTAAG |
| | | complete cds | AATAACGTACAACAATAGGCTACTTCTGCCAAGT |
| | | | GCTGAGCTAAAGCCATCCGCGGTTGTAAGTAAAC |
| | | | CACGAGTCGATATCGGTGGCAGTGACATGAGGGC |
| | | | TTTCTACACCCTGGTACTGATTGACCCGGATGCCC |
| | | | CAAGTCCAAGCCATCCATCACTAAGGGAGTACTT |
| | | | GCACTGGATGGTGACAGATATTCCAGAAACAACT |
| | | | AGTGTCAACTTTGGCCAAGAGCTAATATTTTATGA |
| | | | GAGGCCGGACCCAAGATCTGGCATCCACAGGCTG |
| | | | GTATTTGTGCTGTTCCGTCAACTTGGCAGGGGGAC |
| | | | AGTTTTTGCACCAGAAATGCGCCACAACTTCAACT |
| | | | GCAGAAGCTTTGCACGGCAATATCACCTCAGCATT |
| | | | GCCACCGCTACACATTTCAACTGTCAAAGGGAAG |
| | | | GTGGATCCGGCGGAAGAAGGTTTAGGGAAGAGTA |
| | | | GAAACCATAGGCCACTGCATGGTCACACTATAGA |
| | | | AATATCATCAATAATGTGCACTATATTGAATCAAT |
| | | | GCACCACCTCTATATGCTGAATGTTATGTATCTCA |
| | | | AACTATGATTGTACTGACTTGAAAGGTTGAGAGCT |
| | | | TAGTCTCTTAGCAGAATATAGCACAATATTACTAG |
| | | | TA |
| 4 | DNA | GmFT2a CDS, | ATGCCTAGTGGAAGTAGGGATCCTCTCGTTGTTGG |
| | | the soy FT | GGGAGTAATTGGGGATGTATTGGATCCTTTTGAAT |
| | | ortholog | ATTCTATTCCTATGAGGGTTACCTACAATAACAGA |
| | | according to | GATGTCAGCAATGGATGTGAATTCAAACCCTCAC |
| | | Sun et al., 2011 | AAGTTGTCAACCAACCAAGGGTAAATATCGGTGG |
| | | and Cai et al., | TGATGACCTCAGGAACTTCTATACTTTGATTGCGG |
| | | 2018 (GenBank | TTGATCCCGATGCACCTAGCCCAAGTGACCCCAAT |
| | | ID: EU287455) | TTGAGAGAATACCTCCATTGGTTGGTGACTGATAT |
| | | | CCCAGCAACAACAGGGGCTAGTTTCGGCCATGAG |
| | | | GTTGTAACATATGAAAGTCCAAGACCAATGATGG |
| | | | GGATTCATCGTTTGGTGTTTGTGTTATTTCGTCAAC |
| | | | TGGGTAGGGAGACCGTGTATGCACCAGGATGGCG |
| | | | CCAGAATTTCAACACTAAAGAATTTGCTGAACTTT |
| | | | ACAACCTTGGATTGCCAGTTGCTGCTGTCTATTTC |
| | | | AACATTCAGAGGGAATCTGGTTCTGGTGGAAGGA |
| | | | GGTTATACTAA |
| 5 | RNA | RNA encoded | AUGUCUAUAAAUAUAAGAGACCCUCUUAUAGUA |
| | | by SEQ ID | AGCAGAGUUGUUGGAGACGUUCUUGAU |
| | | NO: 1 | CCGUUUAAUAGAUCAAUCACUCUAAAGGUUACU |
| | | | UAUGGCCAAA |
| 6 | RNA | RNA encoded | AGUUAAUGCAAAUCCGAAACAGUAUAAAUAUGU |
| | | by SEQ ID | GUAGAGGGUUCAUGCCUAUGAUACAAAUUAAAG |
| | | NO: 2 | AAGCAGAAACAAAAACAAGUAAAACAGAAACAA |
| | | | UCAACACAGAGAAACCACCUGUUUGUUCAAGAU |
| | | | CAAAGAUGUCUAUAAAUAUAAGAGACCCUCUUA |
| | | | UAGUAAGCAGAGUUGUUGGAGACGUUCUUGAUC |
| | | | CGUUUAAUAGAUCAAUCACUCUAAAGGUUACUU |
| | | | AUGGCCAAAGAGAGGUGACUAAUGGCUUGGAUC |
| | | | UAAGGCCUUCUCAGGUUCAAAACAAGCCAAGAG |
| | | | UUGAGAUUGGUGGAGAAGACCUCAGGAACUUCU |
| | | | AUACUUUGGUUAUGGUGGAUCCAGAUGUUCCAA |
| | | | GUCCUAGCAACCCUCACCUCCGAGAAUAUCUCCA |
| | | | UUGGUUGGUGACUGAUAUCCCUGCUACAACUGG |
| | | | AACAACCUUUGGCAAUGAGAUUGUGUGUUACGA |
| | | | AAAUCCAAGUCCCACUGCAGGAAUUCAUCGUGU |
| | | | CGUGUUUAUAUUGUUUCGACAGCUUGGCAGGCA |
| | | | AACAGUGUAUGCACCAGGGUGGCGCCAGAACUU |
| | | | CAACACUCGCGAGUUUGCUGAGAUCUACAAUCU |
| | | | CGGCCUUCCCGUGGCCGCAGUUUUCUACAAUUG |
| | | | UCAGAGGGAGAGUGGCUGCGGAGGAAGAAGACU |
| | | | UUAGAUGGCUUCUUCCUUUAUAACCAAUUGAUA |
| | | | UUGCAUACUCUGAUGAGAUUUAUGCAUCUAUAG |
| | | | UAUUUUAAUUUAAUAACCAUUUUAUGAUACGAG |
| | | | UAACGAACGGUGAUGAUGCCUAUAGUAGUUCAA |
| | | | UAUAUAAGUGUGUAAUAAAAAUGAGAGGGGGAG |
| | | | GAAAAUGAGAGUGUUUUACUUAUAUAGUGUGUG |
| | | | AUGCGAUAAUUAUAUUAAUCUACAUGAAAUGAA |
| | | | GUGUUAUAUUUAUACUUUACGUGUAUUCAUUUC |
| | | | UUUUCGAUGCAAAAAUCAGGCAGUGGGAAGAAU |
| | | | CUGCUGUUUUACUUUUG |
| 7 | RNA | RNA encoded | UUGAGAGUUCUAAUAAGAGCAACGGCCAAUACC |
| | | by SEQ ID | AUUAGCGAGUUAUUUUUCUGCAAUAUAUGUCAG |
| | | NO: 3 | CAACCGAUCAUUUGGUUAUGGCUCGUGUCAUAC |
| | | | AGGAUGUAUUGGAUCCCUUUACACCAACCAUUC |
| | | | CACUAAGAAUAACGUACAACAAUAGGCUACUUC |
| | | | UGCCAAGUGCUGAGCUAAAGCCAUCCGCGGUUG |
| | | | UAAGUAAACCACGAGUCGAUAUCGGUGGCAGUG |
| | | | ACAUGAGGGCUUUCUACACCCUGGUACUGAUUG |
| | | | ACCCGGAUGCCCCAAGUCCAAGCCAUCCAUCACU |
| | | | AAGGGAGUACUUGCACUGGAUGGUGACAGAUAU |
| | | | UCCAGAAACAACUAGUGUCAACUUUGGCCAAGA |
| | | | GCUAAUAUUUUAUGAGAGGCCGGACCCAAGAUC |
| | | | UGGCAUCCACAGGCUGGUAUUUGUGCUGUUCCG |
| | | | UCAACUUGGCAGGGGGACAGUUUUUGCACCAGA |
| | | | AAUGCGCCACAACUUCAACUGCAGAAGCUUUGC |
| | | | ACGGCAAUAUCACCUCAGCAUUGCCACCGCUACA |
| | | | CAUUUCAACUGUCAAAGGGAAGGUGGAUCCGGC |
| | | | GGAAGAAGGUUUAGGGAAGAGUAGAAACCAUAG |
| | | | GCCACUGCAUGGUCACACUAUAGAAAUAUCAUC |
| | | | AAUAAUGUGCACUAUAUUGAAUCAAUGCACCAC |
| | | | CUCUAUAUGCUGAAUGUUAUGUAUCUCAAACUA |
| | | | UGAUUGUACUGACUUGAAAGGUUGAGAGCUUAG |
| | | | UCUCUUAGCAGAAUAUAGCACAAUAUUACUAGU |
| | | | A |
| 8 | RNA | RNA encoded | AUGCCUAGUGGAAGUAGGGAUCCUCUCGUUGUU |
| | | by SEQ ID | GGGGGAGUAAUUGGGGAUGUAUUGGAUCCUUUU |
| | | NO: 4 | GAAUAUUCUAUUCCUAUGAGGGUUACCUACAAU |
| | | | AACAGAGAUGUCAGCAAUGGAUGUGAAUUCAAA |
| | | | CCCUCACAAGUUGUCAACCAACCAAGGGUAAAU |
| | | | AUCGGUGGUGAUGACCUCAGGAACUUCUAUACU |
| | | | UUGAUUGCGGUUGAUCCCGAUGCACCUAGCCCA |
| | | | AGUGACCCCAAUUUGAGAGAAUACCUCCAUUGG |
| | | | UUGGUGACUGAUAUCCCAGCAACAACAGGGGCU |
| | | | AGUUUCGGCCAUGAGGUUGUAACAUAUGAAAGU |
| | | | CCAAGACCAAUGAUGGGGAUUCAUCGUUUGGUG |
| | | | UUUGUGUUAUUUCGUCAACUGGGUAGGGAGACC |
| | | | GUGUAUGCACCAGGAUGGCGCCAGAAUUUCAAC |
| | | | ACUAAAGAAUUUGCUGAACUUUACAACCUUGGA |
| | | | UUGCCAGUUGCUGCUGUCUAUUUCAACAUUCAG |
| | | | AGGGAAUCUGGUUCUGGUGGAAGGAGGUUAUAC |
| | | | UAA |
| 9 | RNA | DQ865290.1 | GGGGCUUCAAAAGAGAAUUAGGUCACCUCCCAG |
| | | Cucurbita | CUCGGUUUCGACACGCCAUGCCGAGAAAUCGUG |
| | | maxima | ACCCUCUAGUCGUCGGGAGAGUGAUCGGCGACG |
| | | flowering locus | UCGUCGACUCGUUCUCGAGGUCCAUCUCGAUUA |
| | | T-like 1 (FTL1) | GGGUUGUUUACGACUCGAGGGAAGUUAACAAUG |
| | | | GGUGUGAGCUCAAACCCUCUCAAGCUGUCAACA |
| | | | AGCCAAGAGUUGAGAUUGGUGGCACUGACCUUC |
| | | | GCACCUUCUUCACUUUGGUUAUGGUGGAUCCCG |
| | | | ACGCUCCUAGCCCUAGCGAUCCCAAUCUAAGAG |
| | | | AAUACUUGCAUUGGUUAGUGACCGAUAUUCCAG |
| | | | CUACAACCGAGGCAACCUUUGGACAAGAGAUAG |
| | | | UGUGCUACGAGAAUCCAAGACCAACGGUGGGUA |
| | | | UCCACCGUUUUGUGCUGGUCUUGUUCCGGCAGC |
| | | | UCGGAAGGCAAACGGUGUAUGCUCCUGGGUGGC |
| | | | GCCAGAACUUCAACACCAGACACUUUGCAGAGC |
| | | | UUUACAAUCUUGGUUCGCCAGUCGCCGCCGUCU |
| | | | AUUUCAAUUGCCAAAGGGAAAAUGGCUCCGGUG |
| | | | GAAGGAGAAGAGCCGGCGAUGAAUGUUCAUAAA |
| | | | AACACUUCACUUCACAUUAUAUUAUCAACCAAU |
| | | | AUAUUGUAAUAACAUGGUUCACGUUUCUAUCUA |
| | | | AUAGAUUAUAUAUUUUUAAUAAGUUCGUGAAAA |
| | | | AAAAAA |
| 10 | RNA | mRNA, | GAGACAAUUACGCAUCUUUUCAGCUCUCUCACG |
| | | complete cds | UACUACCAUCCUCUCGACGCCAUGCCGAGAGACC |
| | | DQ865291.1 | GUGACCCUUUGGUCGUUGGGAGAGUCAUCGGCG |
| | | Cucurbita | ACGUUAUCGACUCGUUCACGAAGUCCAUUUCGA |
| | | maxima | UUAGGGCUACUUACAACAACAGGGAAAUUAGCA |
| | | flowering locus | AUGGCUGUGAGCUCAAACCCUCUCAAGUUGUCA |
| | | T-like 2 (FTL2) | ACCAGCCAAGAGUUGAGAUUGGUGGCACUGACC |
| | | mRNA, | UUCGCACCUUCUUCACUUUGGUUAUGGUGGAUC |
| | | complete cds | CUGAUGCUCCUAGCCCUAGUGAUCCUAAUCUAA |
| | | | GGGAAUACUUGCAUUGGUUGGUGACUGAUAUCC |
| | | | CAGCUACAACUGGAGCGAACUUUGGUCAAGAGA |
| | | | UCGUGUGCUAUGAGAGCCCAAGACCCACGGUGG |
| | | | GUAUCCAUCGUCUUGUGCUGGUGUUGUUUCGAC |
| | | | AGCUUGGAAGGCAAACGGUGUACGCUCCUGGGU |
| | | | GGCGCCAGAACUUCAACACAAGAGACUUUGCAG |
| | | | AGCUUUACAAUCUUGGCUUGCCGGUGGCAGCCG |
| | | | UUUAUUUCAAUUGCCAAAGGGAAAGUGGGUCUG |
| | | | GUGGAAGGAGAAGAACCCAAGAUGAUUUCUAAG |
| | | | CCCCACUUCACAUUAAUUAGAUUAAUAUUAUAG |
| | | | CCCCUAUCAUCUAUUAAUCCUACCUUGCUUUUA |
| | | | GAUUAACCUUUAUUUUGAGUACACCCAUGGAUC |
| | | | AUAAAUAAGCCCAAAAUGCAUUCCUAAUAUUGC |
| | | | UCUUAUACUCGUUUCGUAUGAAUCACUGUCUUU |
| | | | UCUUCUUUGUUUUUCUUGUUCGAGUGUUCAUGU |
| | | | UGUGCUUUUUUUUUCGUAUGAAUCAAAGUAGAA |
| | | | GAUCAAGAUUCGAAAAAAAAAAAAAAAAAAAA |
| 11 | RNA | DQ871590.1 | CCCUCUUGUAUUGUAUCGGUGAGGUGUGUGUGA |
| | | Vitis vinifera | UGCCUAGGGAAAGGGAUCCUCUUGUUGUUGGGC |
| | | FT-like protein | GCGUUGUCGGGGAUGUUCUGGACCCCUUUCUCA |
| | | (FT) mRNA, | GGUCCAUCACUCUGAGGGUGACCUACAAUAAUA |
| | | complete cds | GAGAAGUAGCAAAUGGCUGUGAGUUCAGACCCU |
| | | | CUCAGCUAGUCAGCCAACCUAGGGUGGACAUUG |
| | | | GAGGGGAUGACUUGAGGACCUUCUAUACUUUGG |
| | | | UUAUGGUGGACCCUGACGCUCCAAGCCCCAGUA |
| | | | AUCCGAACCUAAGGGAGUACUUACAUUGGUUGG |
| | | | UGACUGAUAUUCCAGCAACUACUGGGGCAAACU |
| | | | UCGGCCAAGAGAUUGUGUGUUAUGAGAGCCCAC |
| | | | GCCCAACAGCUGGGAUUCAUCGCUUUGUUUUUG |
| | | | UAUUGUUUCGCCAACUGGGUAGGCAGACAGUGU |
| | | | AUGCACCAGGGUGGCGCCAAAAUUUCAACACUA |
| | | | GGGACUUUGCUGAGCUUUAUAAUCUUGGUUUGC |
| | | | CUGUUGCUGCUGUUUAUUUUAACUGCCAAAGGG |
| | | | AGGGCGGCUCGGGUGGUCGAAGAUCAUAAUCAA |
| | | | UGGAUUUUGUACGCAACCUUGCGACUUACAAAG |
| | | | GC |
| 12 | RNA | AB161112.1 | AUGCCUAGGGAUAGGGACCCCCUUGUUGUUGGA |
| | | Malus x | CGAGUGGUUGGUGAUGUUUUAGACCCCUUCACA |
| | | domestica | AGGUCUGUUUCUCUGAGGGUGACCUACGGUACU |
| | | MdFT1 mRNA | AAGGAGGUUAACAAUGGUUGUGAGCUCAAACCU |
| | | for flowering | UCUGAAGUUGUCCAACAACCUAGAGCUGAUAUU |
| | | locus T like | GGUGGAGACGAUCUCAGGACUUUCUACACUCUG |
| | | protein, | GUCAUGGUGGAUCCUGAUGCACCCAGCCCAAGU |
| | | complete cds | GACCCCAACCUAAAGGAAUAUUUGCAUUGGUUG |
| | | | GUUACCGAUAUUCCAGCAACUACUGCGGCAAGC |
| | | | UUCGGGCAAGAGAUCGUGUGUUAUGAAAGUCCA |
| | | | CGGCCAACAGUGGGGAUUCAUCGCUUUGUUUUG |
| | | | GUGGUGUUUCGCCAAUUGGGUAGGCAAACGGUG |
| | | | UAUGCUCCGGGAUGGCGCCAGAACUUCAAUACC |
| | | | AGAGACUUCGCCGAGCUUUAUAAUCUUGGAUUA |
| | | | CCGGUGUCUGUCGUCUAUUUUAACUGCCAAAGG |
| | | | GAGGGCGGCUCCGGUGGAAGGAGAAGAUAA |
| 13 | RNA | AB027456.1 | GGCACGAGGAAUAGUCUUACUACUUUUGUAGGC |
| | | Citrus unshiu | UGUGUGUGUAUUUGUUUGUGCUUAGUGUUGUUG |
| | | CiFT mRNA, | AGUGUUUGUUUGUGUUUAGUGUUGUUGAUAUGU |
| | | complete cds | CUAGCAGGGAGAGAGAUCCUCUUAUUGUUGGCC |
| | | | GCGUUGUUGGUGAUGUUCUUGACAAUUUUACAA |
| | | | GAACAAUUCCAAUGAGGAUUACCUAUUCAAACA |
| | | | AGGAUGUUAAUAAUGGCCGUGAGCUCAAACCUU |
| | | | CUGAAGUUCUGAACCAGCCUAGGGCUGAAAUUG |
| | | | GUGGUGAUGAUCUUAGGACAUUUUAUACUUUGG |
| | | | UAAUGGUUGAUCCUGAUGCACCAAGCCCAAGUG |
| | | | ACCCCAGCCUUAGGGAGUAUUUGCAUUGGUUGG |
| | | | UGACUGAUAUUCCAGCAACCACAGGGGCCAGCU |
| | | | UUGGCCAAGAGAUUGUGAACUAUGAAAGCCCUA |
| | | | GGCCAACGAUGGGGAUUCACAGGUUUGUCUUUG |
| | | | UGUUGUUCCGGCAACUUGGGAGGCAGACUGUUU |
| | | | AUGCACCAGGGUGGCGUCAGAACUUCAGCACGA |
| | | | GGGAUUUUGCUGAGCUUUACAAUCUGGGACCUC |
| | | | CGGUGGCCGCUGUCUACUUCAACUGCCAGAGGG |
| | | | AGAGCGGAUCCGGCGGAAGGCCUGUCAGACGAU |
| | | | GAUCCAUACAUGCUUAAUUUGAUAUCAAAUUAC |
| | | | ACACACACACACACACACACACACACACACACAC |
| | | | ACACACACACACACACACUAUUUAUAUAUAUAU |
| | | | AUAUAUAUAUAUAUAUAU |
| 14 | RNA | AY186735.1: | AUGCCUAGAGAACGUGAUCCUCUUGUUGUUGGU |
| | | 2002-2199, | CGUGUGGUAGGGGAUGUAUUGGACCCUUUCACA |
| | | 2287-2348, | AGAACUAUUGGCCUAAGAGUUAUAUAUAGAGAU |
| | | 4490-4530, | AGAGAAGUUAAUAAUGGAUGCGAGCUUAGGCCU |
| | | 5586-5818 | UCCCAAGUUAUUAACCAGCCAAGGGUUGAAGUU |
| | | Lycopersicon | GGAGGAGAUGACCUACGUACCUUUUUCACUUUG |
| | | esculentum | GUUAUGGUGGACCCUGAUGCUCCAAGUCCGAGU |
| | | SP3D (SP3D) | GAUCCAAAUCUGAGAGAAUACCUUCACUGGUUG |
| | | gene, complete | GUCACCGAUAUUCCAGCUACCACAGGUUCAAGU |
| | | cds | UUUGGGCAAGAAAUAGUGAGCUAUGAAAGUCCA |
| | | | AGACCAUCAAUGGGAAUACAUCGAUUUGUAUUU |
| | | | GUAUUAUUCAGACAAUUAGGUCGGCAAACAGUG |
| | | | UAUGCUCCAGGAUGGCGUCAGAAUUUCAACACA |
| | | | AGAGAUUUUGCAGAACUUUAUAAUCUUGGUUUA |
| | | | CCUGUUGCUGCUGUCUAUUUUAAUUGUCAAAGA |
| | | | GAGAGUGGCAGUGGUGGACGUAGAAGAUCUGCU |
| | | | GAUUGA |
| 15 | RNA | DQ387859.1 | AUGUCAAGGGACAGAGAUCCUCUGAGCGUUGGC |
| | | Populus tremula | CGUGUUAUAGGGGACGUGCUGGACCCCUUCACA |
| | | flowering locus | AAGUCUAUCCCGCUCAGGGUCACCUACAACUCCA |
| | | T-like protein | GAGAGGUCAACAAUGGUUGCGAGCUCAAACCCU |
| | | FT1 (FT1) | CUCAGGUUGCCAACCAGCCGAGGGUUGAUAUUG |
| | | mRNA, | GCGGGGAAGAUCUAAGGACCUUCUACACUCUGG |
| | | complete cds | UUAUGGUGGACCCUGAUGCACCCAGCCCAAGUG |
| | | | ACCCCAGCCUCAGAGAAUAUUUGCAUUGGUUGG |
| | | | UGACUGAUAUUCCAGCAACAACGGGGGCAAGCU |
| | | | UUGGCCAUGAAACUGUGUGCUAUGAGAGCCCGA |
| | | | GGCCGACGAUGGGGAUUCAUCGGUUUGUUUUCG |
| | | | UCUUGUUCCGGCAACUGGGCAGGCAAACUGUGU |
| | | | AUGCCCCUGGGUGGCGCCAGAACUUCAACACCA |
| | | | GAGACUUUGCUGAGGUCUACAAUCUUGGAUCGC |
| | | | CGGUGGCUGCUGUUUAUUUCAACUGCCAGAGGG |
| | | | AGAGUGGCUCUGGUGGUAGGAGGCGAUAA |
| 16 | RNA | DQ100327.1: | AUGGCCGGGAGGGACAGGGAUCCGCUGGUUGUC |
| | | 1332-1532, | GGCAGGGUUGUGGGGGACGUGCUGGACCCCUUC |
| | | 1950-2011, | GUCCGAACCACCAACCUCAGGGUGACCUUCGGG |
| | | 2121-2391 | AACAGGGCCGUGUCCAACGGCUGCGAGCUCAAG |
| | | Hordeum | CCGUCCAUGGUCGCCCAGCAGCCGAGGGUGGAG |
| | | vulgare subsp. | GUGGGCGGCAAUGAGAUGAGGACCUUCUACACG |
| | | vulgare FT-like | CUCGUGAUGGUAGACCCAGAUGCUCCAAGUCCU |
| | | protein (FT1) | AGCGACCCCAACCUUAGAGAGUAUCUCCACUGG |
| | | gene, complete | UUGGUGACAGAUAUCCCGGGUACAACUGGGGCG |
| | | cds | UCGUUCGGGCAGGAGGUGAUGUGCUACGAGAGC |
| | | | CCUCGUCCAACCAUGGGGAUCCACCGCUUCGUGC |
| | | | UCGUGCUCUUCCAGCAGCUGGGGCGGCAGACGG |
| | | | UGUACGCCCCCGGGUGGCGCCAGAACUUCAACAC |
| | | | CAGGGACUUUGCCGAGCUCUACAACCUCGGCCA |
| | | | GCCCGUUGCCGCCGUCUACUUCAACUGCCAGCGC |
| | | | GAGGCCGGCUCCGGCGGCAGGAGGAUGUACAAU |
| | | | UGA |
| 17 | RNA | DQ297407.1: | AUGGUGGGGAGCAGCAUGCAGCGCGGGGACCCG |
| | | 955-1164, | CUGGUGGUGGGGCGGGUGAUCGGCGACGUGGUG |
| | | 1235-1296, | GACCCGUUCGUGCGGCGGGUGGCGCUGCGGGUC |
| | | 3672-3712, | GGCUACGCGUCCAGGGACGUGGCCAACGGCUGC |
| | | 3808-4031 | GAGCUCCGGCCGUCCGCCAUCGCCGACCAGCCGC |
| | | Hordeum | GCGUCGAGGUCGGCGGCCCGGACAUGCGCACCU |
| | | vulgare subsp. | UCUACACCCUGGUGAUGGUGGAUCCGGAUGCUC |
| | | vulgare FT-like | CAAGCCCCAGCGACCCCAGCCUUAGGGAGUACUU |
| | | protein (FT2) | GCACUGGCUGGUCACCGACAUCCCGGCCACGACA |
| | | gene, complete | GGAGUGUCUUUUGGUACCGAGGUUGUGUGCUAC |
| | | cds | GAGGGCCCGCGGCCGGUGCUCGGGAUCCACCGAC |
| | | | UGGUGUUCCUGCUCUUCCAGCAACUCGGCCGAC |
| | | | AGACGGUGUACGCCCCGGGGUGGCGGCAGAACU |
| | | | UCAGCACCCGCGACUUUGCCGAGCUCUACAACCU |
| | | | CGGCCUGCCCGUCGCCGCCGUCUACUUCAACUGC |
| | | | CAGAGGGAGACCGGAACCGGCGGGAGAAGGAUG |
| | | | UGA |
| 18 | RNA | AB052944.1 | UGCACCACACACAGUUCAGCUAGCAGAUCACCU |
| | | Oryza sativa | AGCUAGAUAGCUGCCUCUAUCACAGUAUAUUUG |
| | | Japonica Group | CUCCCUGCAACUUGCUGCUGCUGCAAUAGCUAG |
| | | Hd3a mRNA, | CAGCUGCAGCUAGUAAGCAAAACUAUAAACCUU |
| | | complete cds, | CAGGGUUUUUUGCAAGAUCGAUGGCCGGAAGUG |
| | | cultivar: | GCAGGGACAGGGACCCUCUUGUGGUUGGUAGGG |
| | | Nipponbare | UUGUGGGUGAUGUGCUGGACGCGUUCGUCCGGA |
| | | | GCACCAACCUCAAGGUCACCUAUGGCUCCAAGAC |
| | | | CGUGUCCAAUGGCUGCGAGCUCAAGCCGUCCAU |
| | | | GGUCACCCACCAGCCUAGGGUCGAGGUCGGCGG |
| | | | CAAUGACAUGAGGACAUUCUACACCCUUGUGAU |
| | | | GGUAGACCCAGAUGCACCAAGCCCAAGUGACCC |
| | | | UAACCUUAGGGAGUAUCUACAUUGGUUGGUCAC |
| | | | UGAUAUUCCUGGUACUACUGCAGCGUCAUUUGG |
| | | | GCAAGAGGUGAUGUGCUACGAGAGCCCAAGGCC |
| | | | AACCAUGGGGAUCCACCGGCUGGUGUUCGUGCU |
| | | | GUUCCAGCAGCUGGGGCGUCAGACAGUGUACGC |
| | | | GCCCGGGUGGCGUCAGAACUUCAACACCAAGGA |
| | | | CUUCGCCGAGCUCUACAACCUCGGCUCGCCGGUC |
| | | | GCCGCCGUCUACUUCAACUGCCAGCGCGAGGCAG |
| | | | GCUCCGGCGGCAGGAGGGUCUACCCCUAGCUAA |
| | | | CGAUGAUCCCGAUCGAUCUGCUGCAUGCUCACU |
| | | | AUCAUCAUCCAGCAUGCUAUACAUUGCAGGUUC |
| | | | AGACAAUUGAAAUGAUUCUCGACACACAACAUA |
| | | | UAUAUGAUGGUGUAAUUAAUUAUGCAAUUAAAU |
| | | | AGCUGAGCAAGGCUAAGGU |
| 19 | RNA | AB062676.1 | CCUGUCACUGUUUGGCUAGCUUAACCUUCCUGA |
| | | Oryza sativa | CAUCUAUCCUCUGGAUUGAACGGCAGGAGAUAC |
| | | Japonica Group | CUAAGCUAGCUAGCAAUCUCUAUCGAUCUGUUU |
| | | RFT1 mRNA | GUUUACAUGUUCAGUUAAAGGUUACUGAGAAAU |
| | | for FT-like | GCCUAGAGUUUUUCCGGCUAGCUUCAUAAGUUA |
| | | protein, | GUGGGUUAGCUGACCUAGAUUCAAAGUCUAAUC |
| | | complete cds | CUUUUAUUUAUUUGAUAUUAGAUAUCCUAACGU |
| | | | UUUUAGUUAGAGGUUAUUAAUUUGACAUGGCCG |
| | | | GCAGCGGCAGGGACGAUCCUCUUGUGGUUGGCA |
| | | | GGAUUGUGGGUGAUGUGCUGGAUCCAUUCGUCC |
| | | | GGAUCACUAACCUCAGUGUCAGCUAUGGUGCAA |
| | | | GGAUCGUCUCCAAUGGCUGCGAGCUCAAGCCGU |
| | | | CCAUGGUGACCCAACAGCCCAGGGUCGUGGUCG |
| | | | GUGGCAAUGACAUGAGGACGUUCUACACACUCG |
| | | | UGAUGGUAGACCCGGAUGCUCCGAGCCCAAGCA |
| | | | ACCCUAACCUUAGGGAGUAUCUACACUGGCUGG |
| | | | UCACCGAUAUUCCUGGUACCACUGGAGCAACAU |
| | | | UUGGGCAAGAGGUGAUGUGCUACGAGAGCCCAA |
| | | | GGCCAACCAUGGGGAUCCACCGGCUGGUGUUCG |
| | | | UGCUGUUCCAGCAGCUGGGGCGUCAGACGGUGU |
| | | | ACGCACCGGGGUGGCGCCAGAACUUCAGCACCA |
| | | | GGAACUUCGCCGAGCUCUACAACCUCGGCUCGCC |
| | | | GGUCGCCACCGUCUACUUCAACUGCCAGCGCGAG |
| | | | GCCGGCUCCGGCGGCAGGAGGGUCUACCCCUAGC |
| | | | UAGCUACGCAUGCCACCCGGCCUCCAUGCAUGCA |
| | | | GCAGCUAUAGCUAAGCUGAGACCUGCCUAGCUG |
| | | | UAUA |
| 20 | RNA | EU178859.1 | CACACACACACACAUAUAUAUACAGAGAAAGGU |
| | | Ipomoea nil FT- | UAGUUUUGAUCGAGGAGCUGAGCUAGCUAGGAU |
| | | like protein | GCGAAGGGGAACAGUAGACCCUUUGGUGUUGGG |
| | | (FT1) mRNA, | GCGUGUGAUCGGAGACGUUGUGGAUCCAUUCAC |
| | | complete cds | GAGGUCCGUUGAGCUUAGGGUGGUUUACAAUAA |
| | | | CGAGGUGGAUAUCAGGAAUGGGUGUGAGAUGAG |
| | | | GCCUUCUCAGCUCAUCAACCCACCUAGGGUUGA |
| | | | AAUCGGCGGACACGAUCUCCGUACUUUCUACAC |
| | | | UCUGGUUAUGGUGGAUCCUGAUGCUCCAAGUCC |
| | | | AACCUCUCCAACCCUGAGGGAAUACCUCCACUGG |
| | | | UUGGUCACUGAUAUACCAGGAACUACAGGAGCA |
| | | | AGCUUCGGCAAUGAAGCGAUAUUCUACGAGCCU |
| | | | CCAAGGCCGUCAAUGGGAAUCCACCGUUUUGUG |
| | | | UUUGUGCUUUUCCGGCAACUUGGCCGGCAGACA |
| | | | GUUUAUGCACCGGUUUGGCGCCAGAAUUUCAAC |
| | | | ACUCGAAACUUUGCUGAGAUUUACAAUCUUGGU |
| | | | UUGCCAGUGGCCGUCACUUACUUUAACGGCCAA |
| | | | AGGGAGGGUGGCACCGGCGGUCGAUCUCCGGCA |
| | | | GAGCCCUGGGCAGCCGAUUAAUUACCCUGCUCC |
| | | | UUCCCGUUAAUUUCAUGCAUGCAUGCAUGCUAU |
| | | | CUAUAGCAUAACAUACAUAUAGUAUAUAUCAUA |
| | | | AAUAAAUAAGACCACAUGCAUUAACAUGUUUAA |
| | | | UUUUCCCAUGAAUAUAUGUUAAAGUUGUUCUAG |
| | | | AAGAACUACGUACUCCAUUAUAUUACCCUUUAU |
| | | | AUAUGGCAAUGAAGAUGGUUUCAUCUCUAUUUA |
| | | | GAAGCUAAAAAAAAAAAAAAAA |
| 21 | RNA | AB027506.1 | UUUAUUGAGAUACUUGAGAUCCAAGAUAAAUAU |
| | | Arabidopsis | GUCUUUAGUCGUAGAGAUCCUCUUGUGGUCGGC |
| | | thaliana TSF | AGUGUUGUUGGAGAUGUUCUUGAUCCUUUCACG |
| | | (TWIN SISTER | AGGUUGGUCUCUCUUAAGGUCACUUAUGGCCAU |
| | | OF FT) mRNA, | AGAGAGGUUACUAAUGGCUUGGAUCUAAGGCCU |
| | | complete cds | UCUCAAGUUCUGAACAAACCAAUAGUGGAGAUU |
| | | | GGAGGAGACGACUUCAGAAAUUUCUACACCUUG |
| | | | GUUAUGGUGGAUCCAGAUGUGCCGAGUCCAAGC |
| | | | AACCCUCACCAACGAGAAUAUCUCCACUGGUUG |
| | | | GUGACUGAUAUACCUGCCACCACUGGAAAUGCC |
| | | | UUUGGCAAUGAGGUGGUGUGCUACGAGAGUCCA |
| | | | CGUCCCCCCUCGGGAAUUCAUCGUAUUGUGUUG |
| | | | GUAUUGUUCCGGCAACUCGGAAGACAAACGGUU |
| | | | UAUGCACCGGGGUGGCGCCAACAGUUCAACACU |
| | | | CGUGAGUUUGCUGAGAUCUACAAUCUUGGUCUU |
| | | | CCUGUGGCUGCCUCUUACUUCAACUGCCAGAGG |
| | | | GAGAAUGGCUGUGGGGGAAGAAGAACGUAGAUG |
| | | | CGUACCUACUUACGUUAACUAAUAAUCUAAUCG |
| | | | UAUAAUAUUCCCUUAAUGAAGUAUUUAAGCAUC |
| | | | UAUGUCAAUGUAAUAAGAAUUUAAAGAUACGAG |
| | | | CUAAAAAAAAUGAUGCAUAUGCUGACAUCGAUG |
| | | | UAAAGUAGUUUACACUUUUAAUGUAAUAACUAG |
| | | | GUUUUAACCCGCGGUACACCGCGAGACUAUUUU |
| | | | GUUUUUUUAAGAAUAAAAAUAUAAUUUGUUUAG |
| | | | UCGAUU |
| 22 | RNA | LC128590.1: | AUGGCACGGGAGAACCCUCUUGUUAUUGGUGGU |
| | | 3049-3243, | GUGAUUGGGGAUGUUCUCAACCCUUUUACAAGC |
| | | 3377-3438, | UCCGUUUCUUUGACUGUUUCAAUCAAUAAUAGG |
| | | 3830-3870, | GCGAUUAGCAAUGGCUUGGAACUCAGGCCCUCU |
| | | 4102-4322 | CAAGUUGUUAAUCGCCCUAGGGUUACUGUUGGU |
| | | Glycine max | GGUGAAGACCUAAGGACCUUCUACACUCUGGUU |
| | | FT5a gene for | AUGGUGGAUGCAGAUGCACCUAGCCCUAGCAAC |
| | | flowering locus | CCUGUCUUGAGGGAAUACCUUCACUGGAUGGUG |
| | | T, complete | ACAGAUAUUCCAGCUACCACAAAUGCAAGCUUU |
| | | cds, cultivar: | GGGAGAGAGGUUGUGUUUUAUGAGAGCCCGAAC |
| | | Toyoharuka | CCUUCAGUAGGGAUUCAUCGAAUCGUGUUCGUA |
| | | | UUGUUCCAGCAAUUGGGCAGAGACACUGUCAUC |
| | | | ACCCCAGAAUGGCGCCAUAAUUUCAAUUCCAGA |
| | | | AACUUUGCUGAAAUUAAUAACCUUGCACCUGUU |
| | | | GCAGCAGCUUAUGCCAACUGCCAAAGAGAGCGU |
| | | | GGUUGCGGUGGAAGGAGAUAUUAA |
| 23 | RNA | ZmZCN9 | AGAGCACAUCCGUAGUGUGUGCAUGCAUCACAG |
| | | NM_001112777.2 | UCACACACACACAGCAGAAGAAGAAGAAACCGA |
| | | Zea mays | ACGAGGGUUUAGCUAGCAAAAUAAACAGAAGCA |
| | | ZCN9 protein | AGCAAGCUAGCUAGAGCUAAGGAUCGAGAUCGA |
| | | (LOC100127520), | GAUCGACCGACCGACGACGAUCAGCUAGCAUGG |
| | | mRNA | CGCGCUUCGUGGAUCCGCUGGUGGUGGGGCGGG |
| | | | UGAUCGGCGAGGUGGUGGACCUGUUCGUGCCUU |
| | | | CCAUCUCCAUGACCGUCGCCUAUGAUGGCCCCAA |
| | | | GGACAUCAGCAACGGCUGCCUCCUCAAGCCGUCC |
| | | | GCCACCGCCGCGCCGCCGCUCGUCCGCAUCUCCG |
| | | | GCCGCCGCAACGACCUCUACACGCUGAUCAUGAC |
| | | | GGACCCCGAUGCGCCUAGCCCCAGCAACCCGACC |
| | | | AUGAGGGAGUACCUCCACUGGAUAGUGAUUAAC |
| | | | AUACCAGGAGGAACAGAUGCUACUAAAGGUGAG |
| | | | GAGGUGGUGGAGUACAUGGGCCCGCGGCCGCCG |
| | | | GUGGGUAUCCACCGCUACGUGCUGGUGCUGUUC |
| | | | GAGCAGAAGACGCGCGUGCACGCGGAGGCCCCC |
| | | | GGCGACCGCGCCAACUUCAAGACGCGCGCGUUCG |
| | | | CGGCGGCGCACGAGCUCGGCCUCCCCACUGCCGU |
| | | | CGUCUACUUCAACGCGCAGAAGGAGCCCGCCAGC |
| | | | CGCCGCCGCUAGCUAGCAGCUCCUCUCUGAGGCA |
| | | | UGCCAGAUGCAUGCGUGUGCGUGCAGGUGCAAC |
| | | | CACCGCACUGCCGGCGGCUACGUAUGACCGGUG |
| | | | AAUAAAAAGUUUUACUGCACCGUAAGCAUGCUC |
| | | | GCCCUGUUGCUAUUGGUAUAUGUUAGCAGUGUG |
| | | | GCAGUCUGUAUGUAGUAGCUAUUCGCUUGCAUC |
| | | | UAUGCACUCUAUGUUAGUAUGCGUACGUGUGGU |
| | | | U |
| 24 | RNA | ZmZCN10 | UGGCAAAAACCCAGCGCUUUGUGCCGCCGCCGUC |
| | | >EU241926.1 | CGCCGGCCCCUCUGCCCUUGUACGCGCACCUAGA |
| | | Zea mays | CACAUCGUCAUCGAUCAUCACACGCAAUCGACAC |
| | | ZCN10 | AAGAAGUUAAUAAACAGCCCAAGGACGCAGAGA |
| | | (ZCN10) | UCAGCUGAUCGAGAAGGACUUGUACUACUACUC |
| | | mRNA, | AGUAUUGUCGUCACAUGCACAUAUAUGUACAUA |
| | | complete cds | AAGAGCUAGCUACCUGAGCUCUACCCAAGGUCG |
| | | | CGUUGAUCGAUCGAUCAUGGCGCGGUUCGUGGA |
| | | | CCCGCUGGUGGUGGGGCGGGUGAUCGGCGAGGU |
| | | | GGUGGACCUGUUCGUGCCCUCCGUCUCCAUGACC |
| | | | GUCGCCUAUGGCCCCAAAGACAUCAGCAACGGC |
| | | | UGCCUCCUCAAGCCGUCCGCCACCGCCGCGCCGC |
| | | | CGCUCGUCCGCAUCUCCGGCCGCCGCGACGACCU |
| | | | CUACACGCUGAUCAUGACGGACCCAGAUGCGCC |
| | | | UAGCCCCAGCGACCCGACCAUGAGGGAGUACCUC |
| | | | CACUGGAUAGUGACUAACAUACCAGGAGGAACG |
| | | | GAUGCAAACAAAGGUGAGGAGGUGGUGGAGUAC |
| | | | AUGGGCCCGCGGCCGCCGGUCGGAAUCCACCGCU |
| | | | ACGUGCUGGUGCUGUUCGAGCAGAAGACGCGUG |
| | | | UGCACGCGGAGGGUCCCGGUGAGCGCGCCAACU |
| | | | UCAACACACGCGCGUUCGCGGCGGCGCACGAGCU |
| | | | CGGCCUCCCCACCGCCGUCGUGUACUUCAACGCG |
| | | | CAGAAAGAGCCGGCCAACCACCGCCGCCGCUAGC |
| | | | UAGUAGCUCCAACAAGGGCGCGCCAGCUGAGCU |
| | | | GCGUGCGUGCAACCCACCACACAGCCGCCGGCGA |
| | | | AGGCUGCCUAUAUGACCGGCGAAUAAAAAGUCU |
| | | | UACUGCACCGUCCGUAAGCGUACUCUCUGUUGG |
| | | | UAUAUGCUUGUCUUCAGGCUCUUGAGUCUAUCU |
| | | | ACUUAAAUGUGGUUACCACUGAGUAAUAGAAGC |
| | | | AGUUGGCGCUUCGAUCGAUCAUUCUAAUAUCCG |
| | | | UACGUGUCAAUCAUUCCUGUUUCCAUCAUCUUG |
| | | | CAUUUGAAGACGCAUUGGUUCUACACCAAGGUG |
| | | | U |
| 25 | RNA | CmNACP: | GACUUUUUAUUCAACAAUCUCUCUCUCUCUCUC |
| | | >FJ151402.1 | UCAACUUCCGAUCAAGUCUCUCCGCCGUCUUUUC |
| | | Cucurbita | ACCGGAGCUGACAAUUCCGAUCAUUUUUUGCUU |
| | | maxima NAC- | CCCUUAAAUUUCCGGCAUGGAGGAACCACCGCC |
| | | domain | AAACGCCUUGGAUUUGCCCCCUGGCUUCAGAUU |
| | | containing | CCACCCCACCGACGAGGAGAUCGUCACUUAUUAC |
| | | protein | CUGAUACAUAAGAUCACCGACGCCGCCUUCACU |
| | | (NACP1) | GCCACCGCCAUCGGAGAAGCUGACCUGAAUAAG |
| | | mRNA, | UGUGAACCUUGGGAUUUGCCACAUAAAGCUAAG |
| | | complete cds | AUGGGGGAAAAAGAAUGGUAUUUUUUUUGCCAG |
| | | | AGAGACCGGAAAUAUCCGACCGGGAUGAGAACG |
| | | | AACCGGGCGACUCAGACCGGUUACUGGAAAGCG |
| | | | ACCGGGAAAGACAAGGAGAUUCUCAAGGGAAGA |
| | | | ACGGUUCUGGCUGGUAUGAAGAAAACGCUGGUU |
| | | | UUUUACAAAGGAAGAGCUCCCAAAGGUGAAAAG |
| | | | ACCAAUUGGGUCAUGCAUGAAUUUCGACUCGAA |
| | | | CCCAAAUUCUUUCAGUUUCUUGGUUUUCCCAAG |
| | | | CCCAUUAAGGCUGAUUGGGUUGUAUGUCGGGUU |
| | | | UUUCACAAGAACACAACGAACACGGUCGGAGUA |
| | | | GUGAAAAAGAUUCAAACUUCUGAUUUUUCUUCU |
| | | | UCUCUCCCACCUCUAAUAGAUCCCACAACUGCUC |
| | | | AUACUCCAAUCAGUGGCAGAUUCGAUAAUGGUG |
| | | | AAGUCAACUGGAGGUUAUCAGUACCAUUCGAUA |
| | | | AUUAUGCAAAUGAUUACCAUUAUCAUCGGCCUU |
| | | | UUUCAGCGACGAAUACUGCAGUGACAAUGAUUU |
| | | | CGUCGUACCCAUCGUCUGUCCCCGACGACGAAUU |
| | | | CUUCUCAUUUGAUCAACUAGACGUCGGUGGAAC |
| | | | AAUGUCAAUGGCGGCGGCGACGACAACAACAAC |
| | | | AACAACUAUGGAGUGCAAAAUAGAACAAGUUUC |
| | | | AUGGUCAACGAUGAGCGGUGUGACACCGGAGAU |
| | | | AUCAUCGUCGAUUGACAACGAGGCAGCUCUCGA |
| | | | GUUCUGGGACUACUGAAAAUUGAAAGUAGAUGU |
| | | | UAUGAUCGAACAAUGGCGAUGCUUUGUUUUAAA |
| | | | UGGGCAUUUCCCAUAUUGAACGUUUAAACAAUG |
| | | | AUUAAUUGAUUGCUAAUUAUUAUUAUUUUUUUU |
| | | | UUUUGGUUACAUAGUCCUUUUUGGGAAGGAAUA |
| | | | UUAGAACUUUCAUGGGUUUGGUUUGUUGAUUGU |
| | | | AUUGAUAUGUAGCAAUGUGACAUUGUAUAUAGC |
| | | | UUCUUUAUCUUUUAUUUUAACCGUUGCAAA |
| 26 | RNA | GAI: | UAAUAAUCAUUUUUUUUCUUAUAACCUUCCUCU |
| | | >Y15193.1 | CUAUUUUUACAAUUUAUUUUGUUAUUAGAAGUG |
| | | Arabidopsis | GUAGUGGAGUGAAAAAACAAAUCCUAAGCAGUC |
| | | thaliana GAI | CUAACCGAUCCCCGAAGCUAAAGAUUCUUCACC |
| | | gene | UUCCCAAAUAAAGCAAAACCUAGAUCCGACAUU |
| | | | GAAGGAAAAACCUUUUAGAUCCAUCUCUGAAAA |
| | | | AAAACCAACCAUGAAGAGAGAUCAUCAUCAUCA |
| | | | UCAUCAAGAUAAGAAGACUAUGAUGAUGAAUGA |
| | | | AGAAGACGACGGUAACGGCAUGGAUGAGCUUCU |
| | | | AGCUGUUCUUGGUUACAAGGUUAGGUCAUCGGA |
| | | | AAUGGCUGAUGUUGCUCAGAAACUCGAGCAGCU |
| | | | UGAAGUUAUGAUGUCUAAUGUUCAAGAAGACGA |
| | | | UCUUUCUCAACUCGCUACUGAGACUGUUCACUA |
| | | | UAAUCCGGCGGAGCUUUACACGUGGCUUGAUUC |
| | | | UAUGCUCACCGACCUUAAUCCUCCGUCGUCUAAC |
| | | | GCCGAGUACGAUCUUAAAGCUAUUCCCGGUGAC |
| | | | GCGAUUCUCAAUCAGUUCGCUAUCGAUUCGGCU |
| | | | UCUUCGUCUAACCAAGGCGGCGGAGGAGAUACG |
| | | | UAUACUACAAACAAGCGGUUGAAAUGCUCAAAC |
| | | | GGCGUCGUGGAAACCACCACAGCGACGGCUGAG |
| | | | UCAACUCGGCAUGUUGUCCUGGUUGACUCGCAG |
| | | | GAGAACGGUGUGCGUCUCGUUCACGCGCUUUUG |
| | | | GCUUGCGCUGAAGCUGUUCAGAAGGAGAAUCUG |
| | | | ACUGUGGCGGAAGCUCUGGUGAAGCAAAUCGGA |
| | | | UUCUUAGCUGUUUCUCAAAUCGGAGCUAUGAGA |
| | | | AAAGUCGCUACUUACUUCGCCGAAGCUCUCGCG |
| | | | CGGCGGAUUUACCGUCUCUCUCCGUCGCAGAGU |
| | | | CCAAUCGACCACUCUCUCUCCGAUACUCUUCAGA |
| | | | UGCACUUCUACGAGACUUGUCCUUAUCUCAAGU |
| | | | UCGCUCACUUCACGGCGAAUCAAGCGAUUCUCG |
| | | | AAGCUUUUCAAGGGAAGAAAAGAGUUCAUGUCA |
| | | | UUGAUUUCUCUAUGAGUCAAGGUCUUCAAUGGC |
| | | | CGGCGCUUAUGCAGGCUCUUGCGCUUCGACCUG |
| | | | GUGGUCCUCCUGUUUUCCGGUUAACCGGAAUUG |
| | | | GUCCACCGGCACCGGAUAAUUUCGAUUAUCUUC |
| | | | AUGAAGUUGGGUGUAAGCUGGCUCAUUUAGCUG |
| | | | AGGCGAUUCACGUUGAGUUUGAGUACAGAGGAU |
| | | | UUGUGGCUAACACUUUAGCUGAUCUUGAUGCUU |
| | | | CGAUGCUUGAGCUUAGACCAAGUGAGAUUGAAU |
| | | | CUGUUGCGGUUAACUCUGUUUUCGAGCUUCACA |
| | | | AGCUCUUGGGACGACCUGGUGCGAUCGAUAAGG |
| | | | UUCUUGGUGUGGUGAAUCAGAUUAAACCGGAGA |
| | | | UUUUCACUGUGGUUGAGCAGGAAUCGAACCAUA |
| | | | AUAGUCCGAUUUUCUUAGAUCGGUUUACUGAGU |
| | | | CGUUGCAUUAUUACUCGACGUUGUUUGACUCGU |
| | | | UGGAAGGUGUACCGAGUGGUCAAGACAAGGUCA |
| | | | UGUCGGAGGUUUACUUGGGUAAACAGAUCUGCA |
| | | | ACGUUGUGGCUUGUGAUGGACCUGACCGAGUUG |
| | | | AGCGUCAUGAAACGUUGAGUCAGUGGAGGAACC |
| | | | GGUUCGGGUCUGCUGGGUUUGCGGCUGCACAUA |
| | | | UUGGUUCGAAUGCGUUUAAGCAAGCGAGUAUGC |
| | | | UUUUGGCUCUGUUCAACGGCGGUGAGGGUUAUC |
| | | | GGGUGGAGGAGAGUGACGGCUGUCUCAUGUUGG |
| | | | GUUGGCACACACGACCGCUCAUAGCCACCUCGGC |
| | | | UUGGAAACUCUCCACCAAUUAGAUGGUGGCUCA |
| | | | AUGAAUUGAUCUGUUGAACCGGUUAUGAUGAUA |
| | | | GAUUUCCGACCGAAGCCAAACUAAAUCCUACUG |
| | | | UUUUUCCCUUUGUCACUUGUUAAGAUCUUAUCU |
| | | | UUCAUUAUAUUAGGUAAUUGAAAAAUUUCUAAA |
| | | | UUACUCACACUGGC |
| 27 | RNA | LeT6 a tomato | AAAGAAAAAAGGAAUAUUGUGUGUUUGCUUUUU |
| | | KNOX gene: | UUUCUGACUAGUAGUAUUGCUAACUAUGUAUUC |
| | | >AF000141.1 | CAUUAAGGAUUUGCUGUGAAAAAGCCUGAUAUC |
| | | Lycopersicon | AGUAAGCAUAAAACUCGGGAGAUCACUUACACA |
| | | esculentum | CACACACACCCUCCUAAAAAAGAGAAGAGAGAU |
| | | class I knotted- | UUACUGUUAAACAGAGGUUUUUUUCCAUUUCUU |
| | | like | UUUUUUUUUCAGUGUGUGUGUGAGAGAAAGAGA |
| | | homeodomain | UGAUUUUCAUAGGCACAAACAAAUAGAAAGGAA |
| | | protein (LeT6) | CAAAAUUUAGAGUGAAGAAGAAAGUGUGUGAGA |
| | | mRNA, | GAAUAAUGGAGGGUGGUUCUAGUGGAAAUACUA |
| | | complete cds | GUACAUCUUGUUUAAUGAUGAUGGGAUAUGGAG |
| | | | AUCAUGAAAACAACAACAACAACAAUGGAAAUG |
| | | | GUAAUGGAAAUGGAAAUGGAAAUGUAACAAUUU |
| | | | GUGCUCCUCCAAUGAUGAUGAUGAUGCCUCCUC |
| | | | CUCCUCCUUCUUUAACUAACAAUAACAAUGCAG |
| | | | AAACAAGCAACAACAACAUCCUUUUUCUUCCUU |
| | | | UCAUGGACAACAACAACAACAAUAAUCCUCAAG |
| | | | AAGACAACAACUCUUCUUCUUCUUCCAUCAAGU |
| | | | CAAAGAUUAUGGCUCAUCCUCACUACCAUCGUC |
| | | | UCUUGACUGCUUAUCUCAAUUGUCAAAAGAUAG |
| | | | GAGCUCCGCCAGAAGUGGUGGCAAGGCUAGAGG |
| | | | AAAUAUGUGCCACGUCAGCAACAAUGGGCCGUA |
| | | | GCAGUAGUAGUAGUGGUGGUGGAAUCAUUGGAG |
| | | | AAGAUCCUGCACUAGAUCAGUUCAUGGAGGCUU |
| | | | AUUGUGAGAUGCUGACAAAAUAUGAACAAGAAC |
| | | | UCUCAAAACCCUUCAAGGAAGCCAUGGUUUUUC |
| | | | UUUCAAGAAUUGAGUGUCAGUUCAAAGCUUUAA |
| | | | CUCUUGCACCUAAUUCUUCUCAUGAAUCUGCUU |
| | | | UGGGCGAGGCAAUGGAUAGAAAUGGAUCAUCUG |
| | | | AUGAAGAGGUUGACGUGAAUAACAGUUUCAUCG |
| | | | ACCCCCAGGCUGAGGAUAGAGAGCUCAAAGGUC |
| | | | AAUUGUUGCGUAAGUACAGCGGUUACUUGGGAA |
| | | | GCCUUAAGCAGGAGUUCAUGAAGAAGAGGAAGA |
| | | | AAGGCAAGCUGCCUAAGGAAGCAAGGCAACAAU |
| | | | UGGUGGAUUGGUGGCUUAGACAUAUUAAAUGGC |
| | | | CAUAUCCAUCGGAAUCUCAGAAGCUUGCACUAG |
| | | | CUGAAUCAACGGGAUUGGACCAGAAGCAAAUAA |
| | | | ACAACUGGUUUAUCAAUCAAAGAAAGAGGCAUU |
| | | | GGAAACCAUCAGAAGAUAUGCAGUUUGUUGUGA |
| | | | UGGAUGCUGCUCAUCCACAUUACUAUAUGGAUA |
| | | | AUGUUCUUGCUAACCAUUUCCCAAUGGAUAUGA |
| | | | CACCCUCUCUCCUCUGAAUUAAGAUUUGUCAUU |
| | | | AUUAAUAUCAAGGAUGUUUAAUUAAUUUGCAUA |
| | | | UUACUUGUGUGCAUGUAGUAGUACAAGCUAUUG |
| | | | UGACACAAUCAACUUUUUAUUAGACCAAAUAUA |
| | | | UAAAGUGCUUGUAAUAGAUCUUUCUAUUAUCAU |
| | | | CUUUAAUUAUGGAAUUAAAUAGUUUGUACUUGC |
| | | | UAAAA |
| 28 | RNA | BEL5: | CAUGCAGAGAUAAAAAUAUAGAUCAGUCUGACA |
| | | >NM_001287992.1 | AGAAGGCAACUUCUCAAAGCUUAGAGAGCUACC |
| | | Solanum | ACCCGAAGAUAGACAGUUAGUUACAUGUACUGU |
| | | tuberosum | UAUAGAUAAAAGGAGAAAUCCGAAGAAGAAAGA |
| | | BEL1-related | AUUUUUUUUGCAGAUAUGUACUAUCAAGGAACC |
| | | homeotic | UCGGAUAAUACUAAUAUACAAGCUGAUCAUCAA |
| | | protein 5 | CAACGUCAUAAUCAUGGGAAUAGUAAUAAUAAU |
| | | (BEL5), mRNA | AAUAUUCAGACACUUUAUUUGAUGAACCCUAAC |
| | | | AAUUAUAUGCAAGGCUACACUACUUCUGACACA |
| | | | CAGCAGCAGCAGCAGUUACUUUUCCUGAAUUCU |
| | | | UCACCAGCAGCAAGCAACGCGCUUUGCCAUGCG |
| | | | AAUAUACAACACGCGCCGCUGCAACAGCAGCAC |
| | | | UUUGUCGGUGUGCCUCUUCCGGCAGUAAGUUUG |
| | | | CACGAUCAGAUCAAUCAUCAUGGACUUUUACAG |
| | | | CGCAUGUGGAACAACCAAGAUCAAUCUCAGCAG |
| | | | GUGAUAGUACCAUCGUCGACGGGGGUUUCUGCC |
| | | | ACGUCAUGUGGCGGGAUCACCACGGACUUGGCG |
| | | | UCUCAAUUGGCGUUUCAGAGGCCGAUUCCGACA |
| | | | CCACAACACCGACAGCAGCAACAACAGCAAGGCG |
| | | | GUCUAUCUCUAAGCCUUUCUCCUCAGCUACAAC |
| | | | AGCAAAUUAGUUUCAAUAACAAUAUUUCAUCCU |
| | | | CAUCACCAAGGACAAAUAAUGUUACUAUUAGGG |
| | | | GAACAUUAGAUGGAAGUUCUAGCAACAUGGUUU |
| | | | UAGGCUCUAAGUAUCUGAAAGCUGCACAAGAGC |
| | | | UUCUUGAUGAAGUUGUUAAUAUUGUUGGAAAAA |
| | | | GCAUCAAAGGAGAUGAUCAAAAGAAGGAUAAUU |
| | | | CAAUGAAUAAAGAAUCAAUGCCUUUGGCUAGUG |
| | | | AUGUCAACACUAAUAGUUCUGGUGGUGGUGAAA |
| | | | GUAGCAGCAGGCAGAAAAAUGAAGUUGCUGUUG |
| | | | AGCUUACAACUGCUCAAAGACAAGAACUUCAAA |
| | | | UGAAAAAAGCCAAGCUUCUUGCCAUGCUUGAAG |
| | | | AGGUGGAGCAAAGGUACAGACAGUACCAUCACC |
| | | | AAAUGCAAAUAAUUGUAUUAUCAUUUGAGCAAG |
| | | | UAGCAGGAAUUGGAUCAGCCAAAUCAUACACUC |
| | | | AAUUAGCUUUGCAUGCAAUUUCGAAGCAAUUCA |
| | | | GAUGCCUAAAGGAUGCAAUUGCUGAGCAAGUAA |
| | | | AGGCGACGAGCAAGAGUUUAGGUGAAGAGGAAG |
| | | | GCUUGGGAGGGAAAAUCGAAGGCUCAAGACUCA |
| | | | AAUUUGUGGACCAUCAUCUAAGGCAACAACGCG |
| | | | CGCUGCAACAGAUAGGAAUGAUGCAACCAAAUG |
| | | | CUUGGAGACCCCAAAGAGGUUUACCUGAAAGAG |
| | | | CUGUCUCUGUCCUUCGUGCUUGGCUUUUCGAGC |
| | | | AUUUUCUUCAUCCUUACCCAAAGGAUUCAGACA |
| | | | AAAUCAUGCUUGCUAAGCAAACGGGGCUAACAA |
| | | | GGAGCCAGGUGUCUAACUGGUUCAUAAAUGCUC |
| | | | GAGUUCGAUUAUGGAAGCCAAUGGUAGAAGAAA |
| | | | UGUACUUGGAAGAAGUGAAGAAUCAAGAACAAA |
| | | | ACAGUACUAAUACUUCAGGAGAUAACAAAAACA |
| | | | AAGAGACCAAUAUAAGUGCUCCAAAUGAAGAGA |
| | | | AACAUCCAAUUAUUACUAGCAGCUUAUUACAAG |
| | | | AUGGUAUUACUACUACUCAAGCAGAAAUUUCUA |
| | | | CCUCAACUAUUUCAACUUCCCCUACUGCAGGUGC |
| | | | UUCACUUCAUCAUGCUCACAAUUUCUCCUUCCU |
| | | | UGGUUCAUUCAACAUGGAUAAUACUACUACUAC |
| | | | UGUUGAUCAUAUUGAAAACAACGCGAAAAAGCA |
| | | | AAGAAAUGACAUGCACAAGUUUUCUCCAAGUAG |
| | | | UAUUCUUUCAUCUGUUGACAUGGAAGCCAAAGC |
| | | | UAGAGAAUCAUCAAAUAAAGGGUUUACUAAUCC |
| | | | UUUAAUGGCAGCAUACGCGAUGGGAGAUUUUGG |
| | | | AAGGUUUGAUCCUCAUGAUCAACAAAUGACCGC |
| | | | GAAUUUUCAUGGAAAUAAUGGUGUCUCUCUUAC |
| | | | UUUAGGACUUCCUCCUUCUGAAAACCUAGCCAU |
| | | | GCCAGUGAGCCAACAAAAUUACCUUUCUAAUGA |
| | | | CUUGGGAAGUAGGUCUGAAAUGGGGAGUCAUUA |
| | | | CAAUAGAAUGGGAUAUGAAAACAUUGAUUUUCA |
| | | | GAGUGGGAAUAAGCGAUUUCCGACUCAACUAUU |
| | | | ACCAGAUUUUGUUACAGGUAAUCUAGGAACAUG |
| | | | AAUACCAGAAAGUCUCGUAUUGAUAGCUGAAAA |
| | | | GAUAAAAGGAAGUUAGGGAUACUCUUAUAUUGU |
| | | | GUGAGGCCUUCUGGCCCAAGUCGGAGGACCCAA |
| | | | UUUGAUACAACCUAUCAUAGGAGAAAAGAAGUG |
| | | | GAGACUAAAUUAAAGUAACAAAAUUUUAAAGCA |
| | | | CACUUUCUAGUAUAUAUACUUCUUUUUUUUAUA |
| | | | GUAUAGAAAAGAAGAGAUUUUGUGCUUUAGUGU |
| | | | AUAGAUAGAGUCUACUUAGUAUAGGUUAUACUU |
| | | | CUAGUUCCUUGAGAAGAUUGAUACAACUAGUAG |
| | | | UAUUUUUUUUCUUUUGGGUUGGCUUGGAGUACU |
| | | | AUUUUAAGUUAUUGGAAACUAGCUAUAGUAAAU |
| | | | GUUGUAAAGUUGUGAUAUUGUUCCUCUCAAUUU |
| | | | GCAUAUAAUUUGAAAUAUUUUGUACCUACUAGC |
| | | | UAGUCUCUAAAUUAUGUUUCCAUUGCUUGUAAU |
| | | | UGCAAUUUUAUUUGAAUUUUGUGCUAUCAUUAU |
| | | | UAGAUUAGCAAAAAAAAAAAAAAAAAA |
| 29 | RNA | AT5G57885.1 | AUCAGAGUGGCGCAGCGGAAGCGUGGUGGGCCC |
| | | (tRNA-Met) | AUAACCCACAGGUCCCAGGAUCGAAAC |
| | | | CUGGCUCUGAUA |
| 30 | RNA | AT1G71700 | GCACCAGUGGUCUAGUGGCAUGAUAGUACCCUG |
| | | (tRNA-Gly) | CCACGGUACAGACCCGGGUUCAAUUCC |
| | | | CGGCUGGUGCA |
| 31 | DNA | DQ865290.1 | GGGGCTTCAAAAGAGAATTAGGTCACCTCCCAGC |
| | | Cucurbita | TCGGTTTCGACACGCCATGCCGAGAAATCGTGAC |
| | | maxima | CCTCTAGTCGTCGGGAGAGTGATCGGCGACGTCG |
| | | flowering locus | TCGACTCGTTCTCGAGGTCCATCTCGATTAGGGTT |
| | | T-like 1 (FTL1) | GTTTACGACTCGAGGGAAGTTAACAATGGGTGTG |
| | | mRNA, | AGCTCAAACCCTCTCAAGCTGTCAACAAGCCAAG |
| | | complete cds | AGTTGAGATTGGTGGCACTGACCTTCGCACCTTCT |
| | | | TCACTTTGGTTATGGTGGATCCCGACGCTCCTAGC |
| | | | CCTAGCGATCCCAATCTAAGAGAATACTTGCATTG |
| | | | GTTAGTGACCGATATTCCAGCTACAACCGAGGCA |
| | | | ACCTTTGGACAAGAGATAGTGTGCTACGAGAATC |
| | | | CAAGACCAACGGTGGGTATCCACCGTTTTGTGCTG |
| | | | GTCTTGTTCCGGCAGCTCGGAAGGCAAACGGTGT |
| | | | ATGCTCCTGGGTGGCGCCAGAACTTCAACACCAG |
| | | | ACACTTTGCAGAGCTTTACAATCTTGGTTCGCCAG |
| | | | TCGCCGCCGTCTATTTCAATTGCCAAAGGGAAAAT |
| | | | GGCTCCGGTGGAAGGAGAAGAGCCGGCGATGAAT |
| | | | GTTCATAAAAACACTTCACTTCACATTATATTATC |
| | | | AACCAATATATTGTAATAACATGGTTCACGTTTCT |
| | | | ATCTAATAGATTATATATTTTTAATAAGTTCGTGA |
| | | | AAAAAAAAA |
| 32 | DNA | DQ865291.1 | GAGACAATTACGCATCTTTTCAGCTCTCTCACGTA |
| | | Cucurbita | CTACCATCCTCTCGACGCCATGCCGAGAGACCGTG |
| | | maxima | ACCCTTTGGTCGTTGGGAGAGTCATCGGCGACGTT |
| | | flowering locus | ATCGACTCGTTCACGAAGTCCATTTCGATTAGGGC |
| | | T-like 2 (FTL2) | TACTTACAACAACAGGGAAATTAGCAATGGCTGT |
| | | mRNA, | GAGCTCAAACCCTCTCAAGTIGTCAACCAGCCAA |
| | | complete cds | GAGTTGAGATTGGTGGCACTGACCTTCGCACCTTC |
| | | | TTCACTTTGGTTATGGTGGATCCTGATGCTCCTAG |
| | | | CCCTAGTGATCCTAATCTAAGGGAATACTTGCATT |
| | | | GGTTGGTGACTGATATCCCAGCTACAACTGGAGC |
| | | | GAACTTTGGTCAAGAGATCGTGTGCTATGAGAGC |
| | | | CCAAGACCCACGGTGGGTATCCATCGTCTTGTGCT |
| | | | GGTGTTGTTTCGACAGCTTGGAAGGCAAACGGTG |
| | | | TACGCTCCTGGGTGGCGCCAGAACTTCAACACAA |
| | | | GAGACTTTGCAGAGCTTTACAATCTTGGCTTGCCG |
| | | | GTGGCAGCCGTTTATTTCAATTGCCAAAGGGAAA |
| | | | GTGGGTCTGGTGGAAGGAGAAGAACCCAAGATGA |
| | | | TTTCTAAGCCCCACTTCACATTAATTAGATTAATA |
| | | | TTATAGCCCCTATCATCTATTAATCCTACCTTGCTT |
| | | | TTAGATTAACCTTTATTTTGAGTACACCCATGGAT |
| | | | CATAAATAAGCCCAAAATGCATTCCTAATATTGCT |
| | | | CTTATACTCGTTTCGTATGAATCACTGTCTTTTCTT |
| | | | CTTTGTTTTTCTTGTTCGAGTGTTCATGTTGTGCTT |
| | | | TTTTTTTCGTATGAATCAAAGTAGAAGATCAAGAT |
| | | | TCGAAAAAAAAAAAAAAAAAAAA |
| 33 | DNA | DQ871590.1 | CCCTCTTGTATTGTATCGGTGAGGTGTGTGTGATG |
| | | Vitis vinifera | CCTAGGGAAAGGGATCCTCTTGTTGTTGGGCGCGT |
| | | FT-like protein | TGTCGGGGATGTTCTGGACCCCTTTCTCAGGTCCA |
| | | (FT) mRNA, | TCACTCTGAGGGTGACCTACAATAATAGAGAAGT |
| | | complete cds | AGCAAATGGCTGTGAGTTCAGACCCTCTCAGCTA |
| | | | GTCAGCCAACCTAGGGTGGACATTGGAGGGGATG |
| | | | ACTTGAGGACCTTCTATACTTTGGTTATGGTGGAC |
| | | | CCTGACGCTCCAAGCCCCAGTAATCCGAACCTAA |
| | | | GGGAGTACTTACATTGGTTGGTGACTGATATTCCA |
| | | | GCAACTACTGGGGCAAACTTCGGCCAAGAGATTG |
| | | | TGTGTTATGAGAGCCCACGCCCAACAGCTGGGAT |
| | | | TCATCGCTTTGTTTTTGTATTGTTTCGCCAACTGGG |
| | | | TAGGCAGACAGTGTATGCACCAGGGTGGCGCCAA |
| | | | AATTTCAACACTAGGGACTTTGCTGAGCTTTATAA |
| | | | TCTTGGTTTGCCTGTTGCTGCTGTTTATTTTAACTG |
| | | | CCAAAGGGAGGGCGGCTCGGGTGGTCGAAGATCA |
| | | | TAATCAATGGATTTTGTACGCAACCTTGCGACTTA |
| | | | CAAAGGC |
| 34 | DNA | AB161112.1 | ATGCCTAGGGATAGGGACCCCCTTGTTGTTGGACG |
| | | Malus x | AGTGGTTGGTGATGTTTTAGACCCCTTCACAAGGT |
| | | domestica | CTGTTTCTCTGAGGGTGACCTACGGTACTAAGGAG |
| | | MdFT1 mRNA | GTTAACAATGGTTGTGAGCTCAAACCTTCTGAAGT |
| | | for flowering | TGTCCAACAACCTAGAGCTGATATTGGTGGAGAC |
| | | locus T like | GATCTCAGGACTTTCTACACTCTGGTCATGGTGGA |
| | | protein, | TCCTGATGCACCCAGCCCAAGTGACCCCAACCTA |
| | | complete cds | AAGGAATATTTGCATTGGTTGGTTACCGATATTCC |
| | | | AGCAACTACTGCGGCAAGCTTCGGGCAAGAGATC |
| | | | GTGTGTTATGAAAGTCCACGGCCAACAGTGGGGA |
| | | | TTCATCGCTTTGTTTTGGTGGTGTTTCGCCAATTGG |
| | | | GTAGGCAAACGGTGTATGCTCCGGGATGGCGCCA |
| | | | GAACTTCAATACCAGAGACTTCGCCGAGCTTTATA |
| | | | ATCTTGGATTACCGGTGTCTGTCGTCTATTTTAACT |
| | | | GCCAAAGGGAGGGCGGCTCCGGTGGAAGGAGAA |
| | | | GATAA |
| 35 | DNA | AB027456.1 | GGCACGAGGAATAGTCTTACTACTTTTGTAGGCTG |
| | | Citrus unshiu | TGTGTGTATTTGTTTGTGCTTAGTGTTGTTGAGTGT |
| | | CiFT mRNA, | TTGTTTGTGTTTAGTGTTGTTGATATGTCTAGCAG |
| | | complete cds | GGAGAGAGATCCTCTTATTGTTGGCCGCGTTGTTG |
| | | | GTGATGTTCTTGACAATTTTACAAGAACAATTCCA |
| | | | ATGAGGATTACCTATTCAAACAAGGATGTTAATA |
| | | | ATGGCCGTGAGCTCAAACCTTCTGAAGTTCTGAAC |
| | | | CAGCCTAGGGCTGAAATTGGTGGTGATGATCTTA |
| | | | GGACATTTTATACTTTGGTAATGGTTGATCCTGAT |
| | | | GCACCAAGCCCAAGTGACCCCAGCCTTAGGGAGT |
| | | | ATTTGCATTGGTTGGTGACTGATATTCCAGCAACC |
| | | | ACAGGGGCCAGCTTTGGCCAAGAGATTGTGAACT |
| | | | ATGAAAGCCCTAGGCCAACGATGGGGATTCACAG |
| | | | GTTTGTCTTTGTGTTGTTCCGGCAACTTGGGAGGC |
| | | | AGACTGTTTATGCACCAGGGTGGCGTCAGAACTTC |
| | | | AGCACGAGGGATTTTGCTGAGCTTTACAATCTGGG |
| | | | ACCTCCGGTGGCCGCTGTCTACTTCAACTGCCAGA |
| | | | GGGAGAGCGGATCCGGCGGAAGGCCTGTCAGACG |
| | | | ATGATCCATACATGCTTAATTTGATATCAAATTAC |
| | | | ACACACACACACACACACACACACACACACACAC |
| | | | ACACACACACACACACACTATTTATATATATATAT |
| | | | ATATATATATATATAT |
| 36 | DNA | AY186735.1: | ATGCCTAGAGAACGTGATCCTCTTGTTGTTGGTCG |
| | | 2002-2199, | TGTGGTAGGGGATGTATTGGACCCTTTCACAAGA |
| | | 2287-2348, | ACTATTGGCCTAAGAGTTATATATAGAGATAGAG |
| | | 4490-4530, | AAGTTAATAATGGATGCGAGCTTAGGCCTTCCCA |
| | | 5586-5818 | AGTTATTAACCAGCCAAGGGTTGAAGTTGGAGGA |
| | | Lycopersicon | GATGACCTACGTACCTTTTTCACTTTGGTTATGGT |
| | | esculentum | GGACCCTGATGCTCCAAGTCCGAGTGATCCAAAT |
| | | SP3D (SP3D) | CTGAGAGAATACCTTCACTGGTTGGTCACCGATAT |
| | | gene, complete | TCCAGCTACCACAGGTTCAAGTTTTGGGCAAGAA |
| | | cds | ATAGTGAGCTATGAAAGTCCAAGACCATCAATGG |
| | | | GAATACATCGATTTGTATTTGTATTATTCAGACAA |
| | | | TTAGGTCGGCAAACAGTGTATGCTCCAGGATGGC |
| | | | GTCAGAATTTCAACACAAGAGATTTTGCAGAACTT |
| | | | TATAATCTTGGTTTACCTGTTGCTGCTGTCTATTTT |
| | | | AATTGTCAAAGAGAGAGTGGCAGTGGTGGACGTA |
| | | | GAAGATCTGCTGATTGA |
| 37 | DNA | DQ387859.1 | ATGTCAAGGGACAGAGATCCTCTGAGCGTTGGCC |
| | | Populus tremula | GTGTTATAGGGGACGTGCTGGACCCCTTCACAAA |
| | | flowering locus | GTCTATCCCGCTCAGGGTCACCTACAACTCCAGAG |
| | | T-like protein | AGGTCAACAATGGTTGCGAGCTCAAACCCTCTCA |
| | | FT1 (FT1) | GGTTGCCAACCAGCCGAGGGTTGATATTGGCGGG |
| | | mRNA, | GAAGATCTAAGGACCTTCTACACTCTGGTTATGGT |
| | | complete cds | GGACCCTGATGCACCCAGCCCAAGTGACCCCAGC |
| | | | CTCAGAGAATATTTGCATTGGTTGGTGACTGATAT |
| | | | TCCAGCAACAACGGGGGCAAGCTTTGGCCATGAA |
| | | | ACTGTGTGCTATGAGAGCCCGAGGCCGACGATGG |
| | | | GGATTCATCGGTTTGTTTTCGTCTTGTTCCGGCAA |
| | | | CTGGGCAGGCAAACTGTGTATGCCCCTGGGTGGC |
| | | | GCCAGAACTTCAACACCAGAGACTTTGCTGAGGT |
| | | | CTACAATCTTGGATCGCCGGTGGCTGCTGTTTATT |
| | | | TCAACTGCCAGAGGGAGAGTGGCTCTGGTGGTAG |
| | | | GAGGCGATAA |
| 38 | DNA | >DQ100327.1: | ATGGCCGGGAGGGACAGGGATCCGCTGGTTGTCG |
| | | 1332-1532, | GCAGGGTTGTGGGGGACGTGCTGGACCCCTTCGT |
| | | 1950-2011, | CCGAACCACCAACCTCAGGGTGACCTTCGGGAAC |
| | | 2121-2391 | AGGGCCGTGTCCAACGGCTGCGAGCTCAAGCCGT |
| | | Hordeum | CCATGGTCGCCCAGCAGCCGAGGGTGGAGGTGGG |
| | | vulgare subsp. | CGGCAATGAGATGAGGACCTTCTACACGCTCGTG |
| | | vulgare FT-like | ATGGTAGACCCAGATGCTCCAAGTCCTAGCGACC |
| | | protein (FT1) | CCAACCTTAGAGAGTATCTCCACTGGTTGGTGACA |
| | | gene, complete | GATATCCCGGGTACAACTGGGGCGTCGTTCGGGC |
| | | cds | AGGAGGTGATGTGCTACGAGAGCCCTCGTCCAAC |
| | | | CATGGGGATCCACCGCTTCGTGCTCGTGCTCTTCC |
| | | | AGCAGCTGGGGCGGCAGACGGTGTACGCCCCCGG |
| | | | GTGGCGCCAGAACTTCAACACCAGGGACTTTGCC |
| | | | GAGCTCTACAACCTCGGCCAGCCCGTTGCCGCCGT |
| | | | CTACTTCAACTGCCAGCGCGAGGCCGGCTCCGGC |
| | | | GGCAGGAGGATGTACAATTGA |
| 39 | DNA | DQ297407.1: | ATGGTGGGGAGCAGCATGCAGCGCGGGGACCCGC |
| | | 955-1164, | TGGTGGTGGGGGGGGTGATCGGCGACGTGGTGGA |
| | | 1235-1296, | CCCGTTCGTGCGGCGGGTGGCGCTGCGGGTCGGC |
| | | 3672-3712, | TACGCGTCCAGGGACGTGGCCAACGGCTGCGAGC |
| | | 3808-4031 | TCCGGCCGTCCGCCATCGCCGACCAGCCGCGCGTC |
| | | Hordeum | GAGGTCGGCGGCCCGGACATGCGCACCTTCTACA |
| | | vulgare subsp. | CCCTGGTGATGGTGGATCCGGATGCTCCAAGCCCC |
| | | vulgare FT-like | AGCGACCCCAGCCTTAGGGAGTACTTGCACTGGC |
| | | protein (FT2) | TGGTCACCGACATCCCGGCCACGACAGGAGTGTC |
| | | gene, complete | TTTTGGTACCGAGGTTGTGTGCTACGAGGGCCCGC |
| | | cds | GGCCGGTGCTCGGGATCCACCGACTGGTGTTCCTG |
| | | | CTCTTCCAGCAACTCGGCCGACAGACGGTGTACG |
| | | | CCCCGGGGTGGCGGCAGAACTTCAGCACCCGCGA |
| | | | CTTTGCCGAGCTCTACAACCTCGGCCTGCCCGTCG |
| | | | CCGCCGTCTACTTCAACTGCCAGAGGGAGACCGG |
| | | | AACCGGCGGGAGAAGGATGTGA |
| 40 | DNA | AB052944.1 | TGCACCACACACAGTTCAGCTAGCAGATCACCTA |
| | | Oryza sativa | GCTAGATAGCTGCCTCTATCACAGTATATTTGCTC |
| | | Japonica Group | CCTGCAACTTGCTGCTGCTGCAATAGCTAGCAGCT |
| | | Hd3a mRNA, | GCAGCTAGTAAGCAAAACTATAAACCTTCAGGGT |
| | | complete cds, | TTTTTGCAAGATCGATGGCCGGAAGTGGCAGGGA |
| | | cultivar: Nippon | CAGGGACCCTCTTGTGGTTGGTAGGGTTGTGGGTG |
| | | bare | ATGTGCTGGACGCGTTCGTCCGGAGCACCAACCTC |
| | | | AAGGTCACCTATGGCTCCAAGACCGTGTCCAATG |
| | | | GCTGCGAGCTCAAGCCGTCCATGGTCACCCACCA |
| | | | GCCTAGGGTCGAGGTCGGCGGCAATGACATGAGG |
| | | | ACATTCTACACCCTTGTGATGGTAGACCCAGATGC |
| | | | ACCAAGCCCAAGTGACCCTAACCTTAGGGAGTAT |
| | | | CTACATTGGTTGGTCACTGATATTCCTGGTACTAC |
| | | | TGCAGCGTCATTTGGGCAAGAGGTGATGTGCTAC |
| | | | GAGAGCCCAAGGCCAACCATGGGGATCCACCGGC |
| | | | TGGTGTTCGTGCTGTTCCAGCAGCTGGGGCGTCAG |
| | | | ACAGTGTACGCGCCCGGGTGGCGTCAGAACTTCA |
| | | | ACACCAAGGACTTCGCCGAGCTCTACAACCTCGG |
| | | | CTCGCCGGTCGCCGCCGTCTACTTCAACTGCCAGC |
| | | | GCGAGGCAGGCTCCGGCGGCAGGAGGGTCTACCC |
| | | | CTAGCTAACGATGATCCCGATCGATCTGCTGCATG |
| | | | CTCACTATCATCATCCAGCATGCTATACATTGCAG |
| | | | GTTCAGACAATTGAAATGATTCTCGACACACAAC |
| | | | ATATATATGATGGTGTAATTAATTATGCAATTAAA |
| | | | TAGCTGAGCAAGGCTAAGGT |
| 41 | DNA | AB062676.1 | CCTGTCACTGTTTGGCTAGCTTAACCTTCCTGACA |
| | | Oryza sativa | TCTATCCTCTGGATTGAACGGCAGGAGATACCTAA |
| | | Japonica Group | GCTAGCTAGCAATCTCTATCGATCTGTTTGTTTAC |
| | | RFT1 mRNA | ATGTTCAGTTAAAGGTTACTGAGAAATGCCTAGA |
| | | for FT-like | GTTTTTCCGGCTAGCTTCATAAGTTAGTGGGTTAG |
| | | protein, | CTGACCTAGATTCAAAGTCTAATCCTTTTATTTATT |
| | | complete cds | TGATATTAGATATCCTAACGTTTTTAGTTAGAGGT |
| | | | TATTAATTTGACATGGCCGGCAGCGGCAGGGACG |
| | | | ATCCTCTTGTGGTTGGCAGGATTGTGGGTGATGTG |
| | | | CTGGATCCATTCGTCCGGATCACTAACCTCAGTGT |
| | | | CAGCTATGGTGCAAGGATCGTCTCCAATGGCTGC |
| | | | GAGCTCAAGCCGTCCATGGTGACCCAACAGCCCA |
| | | | GGGTCGTGGTCGGTGGCAATGACATGAGGACGTT |
| | | | CTACACACTCGTGATGGTAGACCCGGATGCTCCG |
| | | | AGCCCAAGCAACCCTAACCTTAGGGAGTATCTAC |
| | | | ACTGGCTGGTCACCGATATTCCTGGTACCACTGGA |
| | | | GCAACATTTGGGCAAGAGGTGATGTGCTACGAGA |
| | | | GCCCAAGGCCAACCATGGGGATCCACCGGCTGGT |
| | | | GTTCGTGCTGTTCCAGCAGCTGGGGCGTCAGACG |
| | | | GTGTACGCACCGGGGTGGCGCCAGAACTTCAGCA |
| | | | CCAGGAACTTCGCCGAGCTCTACAACCTCGGCTCG |
| | | | CCGGTCGCCACCGTCTACTTCAACTGCCAGCGCGA |
| | | | GGCCGGCTCCGGCGGCAGGAGGGTCTACCCCTAG |
| | | | CTAGCTACGCATGCCACCCGGCCTCCATGCATGCA |
| | | | GCAGCTATAGCTAAGCTGAGACCTGCCTAGCTGT |
| | | | ATA |
| 42 | DNA | EU178859.1 | CACACACACACACATATATATACAGAGAAAGGTT |
| | | Ipomoea nil FT- | AGTTTTGATCGAGGAGCTGAGCTAGCTAGGATGC |
| | | like protein | GAAGGGGAACAGTAGACCCTTTGGTGTTGGGGCG |
| | | (FT1) mRNA, | TGTGATCGGAGACGTTGTGGATCCATTCACGAGGT |
| | | complete cds | CCGTTGAGCTTAGGGTGGTTTACAATAACGAGGT |
| | | AB027506.1 | GGATATCAGGAATGGGTGTGAGATGAGGCCTTCT |
| | | | CAGCTCATCAACCCACCTAGGGTTGAAATCGGCG |
| | | | GACACGATCTCCGTACTTTCTACACTCTGGTTATG |
| | | | GTGGATCCTGATGCTCCAAGTCCAACCTCTCCAAC |
| | | | CCTGAGGGAATACCTCCACTGGTTGGTCACTGATA |
| | | | TACCAGGAACTACAGGAGCAAGCTTCGGCAATGA |
| | | | AGCGATATTCTACGAGCCTCCAAGGCCGTCAATG |
| | | | GGAATCCACCGTTTTGTGTTTGTGCTTTTCCGGCA |
| | | | ACTTGGCCGGCAGACAGTTTATGCACCGGTTTGGC |
| | | | GCCAGAATTTCAACACTCGAAACTTTGCTGAGATT |
| | | | TACAATCTTGGTTTGCCAGTGGCCGTCACTTACTT |
| | | | TAACGGCCAAAGGGAGGGTGGCACCGGCGGTCGA |
| | | | TCTCCGGCAGAGCCCTGGGCAGCCGATTAATTACC |
| | | | CTGCTCCTTCCCGTTAATTTCATGCATGCATGCAT |
| | | | GCTATCTATAGCATAACATACATATAGTATATATC |
| | | | ATAAATAAATAAGACCACATGCATTAACATGTTT |
| | | | AATTTTCCCATGAATATATGTTAAAGTTGTTCTAG |
| | | | AAGAACTACGTACTCCATTATATTACCCTTTATAT |
| | | | ATGGCAATGAAGATGGTTTCATCTCTATTTAGAAG |
| | | | CTAAAAAAAAAAAAAAAA |
| 43 | DNA | Arabidopsis | TTTATTGAGATACTTGAGATCCAAGATAAATATGT |
| | | thaliana TSF | CTTTAGTCGTAGAGATCCTCTTGTGGTCGGCAGTG |
| | | (TWIN SISTER | TTGTTGGAGATGTTCTTGATCCTTTCACGAGGTTG |
| | | OF FT) mRNA, | GTCTCTCTTAAGGTCACTTATGGCCATAGAGAGGT |
| | | complete cds | TACTAATGGCTTGGATCTAAGGCCTTCTCAAGTTC |
| | | | TGAACAAACCAATAGTGGAGATTGGAGGAGACGA |
| | | | CTTCAGAAATTTCTACACCTTGGTTATGGTGGATC |
| | | | CAGATGTGCCGAGTCCAAGCAACCCTCACCAACG |
| | | | AGAATATCTCCACTGGTTGGTGACTGATATACCTG |
| | | | CCACCACTGGAAATGCCTTTGGCAATGAGGTGGT |
| | | | GTGCTACGAGAGTCCACGTCCCCCCTCGGGAATTC |
| | | | ATCGTATTGTGTTGGTATTGTTCCGGCAACTCGGA |
| | | | AGACAAACGGTTTATGCACCGGGGTGGCGCCAAC |
| | | | AGTTCAACACTCGTGAGTTTGCTGAGATCTACAAT |
| | | | CTTGGTCTTCCTGTGGCTGCCTCTTACTTCAACTGC |
| | | | CAGAGGGAGAATGGCTGTGGGGGAAGAAGAACG |
| | | | TAGATGCGTACCTACTTACGTTAACTAATAATCTA |
| | | | ATCGTATAATATTCCCTTAATGAAGTATTTAAGCA |
| | | | TCTATGTCAATGTAATAAGAATTTAAAGATACGA |
| | | | GCTAAAAAAAATGATGCATATGCTGACATCGATG |
| | | | TAAAGTAGTTTACACTTTTAATGTAATAACTAGGT |
| | | | TTTAACCCGCGGTACACCGCGAGACTATTTTGTTT |
| | | | TTTTAAGAATAAAAATATAATTTGTTTAGTCGATT |
| 44 | DNA | LC128590.1: | ATGGCACGGGAGAACCCTCTTGTTATTGGTGGTGT |
| | | 3049-3243, | GATTGGGGATGTTCTCAACCCTTTTACAAGCTCCG |
| | | 3377-3438, | TTTCTTTGACTGTTTCAATCAATAATAGGGCGATT |
| | | 3830-3870, | AGCAATGGCTTGGAACTCAGGCCCTCTCAAGTTGT |
| | | 4102-4322 | TAATCGCCCTAGGGTTACTGTTGGTGGTGAAGACC |
| | | Glycine max | TAAGGACCTTCTACACTCTGGTTATGGTGGATGCA |
| | | FT5a gene for | GATGCACCTAGCCCTAGCAACCCTGTCTTGAGGG |
| | | flowering locus | AATACCTTCACTGGATGGTGACAGATATTCCAGCT |
| | | T, complete | ACCACAAATGCAAGCTTTGGGAGAGAGGTTGTGT |
| | | cds, cultivar: | TTTATGAGAGCCCGAACCCTTCAGTAGGGATTCAT |
| | | Toyoharuka | CGAATCGTGTTCGTATTGTTCCAGCAATTGGGCAG |
| | | | AGACACTGTCATCACCCCAGAATGGCGCCATAAT |
| | | | TTCAATTCCAGAAACTTTGCTGAAATTAATAACCT |
| | | | TGCACCTGTTGCAGCAGCTTATGCCAACTGCCAAA |
| | | | GAGAGCGTGGTTGCGGTGGAAGGAGATATTAA |
| 45 | DNA | ZmZCN9 | AGAGCACATCCGTAGTGTGTGCATGCATCACAGT |
| | | NM_001112777.2 | CACACACACACAGCAGAAGAAGAAGAAACCGAA |
| | | Zea mays | CGAGGGTTTAGCTAGCAAAATAAACAGAAGCAAG |
| | | ZCN9 protein | CAAGCTAGCTAGAGCTAAGGATCGAGATCGAGAT |
| | | (LOC100127520), | CGACCGACCGACGACGATCAGCTAGCATGGCGCG |
| | | mRNA | CTTCGTGGATCCGCTGGTGGTGGGGGGGGTGATC |
| | | | GGCGAGGTGGTGGACCTGTTCGTGCCTTCCATCTC |
| | | | CATGACCGTCGCCTATGATGGCCCCAAGGACATC |
| | | | AGCAACGGCTGCCTCCTCAAGCCGTCCGCCACCG |
| | | | CCGCGCCGCCGCTCGTCCGCATCTCCGGCCGCCGC |
| | | | AACGACCTCTACACGCTGATCATGACGGACCCCG |
| | | | ATGCGCCTAGCCCCAGCAACCCGACCATGAGGGA |
| | | | GTACCTCCACTGGATAGTGATTAACATACCAGGA |
| | | | GGAACAGATGCTACTAAAGGTGAGGAGGTGGTGG |
| | | | AGTACATGGGCCCGCGGCCGCCGGTGGGTATCCA |
| | | | CCGCTACGTGCTGGTGCTGTTCGAGCAGAAGACG |
| | | | CGCGTGCACGCGGAG |
| 46 | DNA | ZmZCN10 | TGGCAAAAACCCAGCGCTTTGTGCCGCCGCCGTCC |
| | | >EU241926.1 | GCCGGCCCCTCTGCCCTTGTACGCGCACCTAGACA |
| | | Zea mays | CATCGTCATCGATCATCACACGCAATCGACACAA |
| | | ZCN10 | GAAGTTAATAAACAGCCCAAGGACGCAGAGATCA |
| | | (ZCN10) | GCTGATCGAGAAGGACTTGTACTACTACTCAGTAT |
| | | mRNA, | TGTCGTCACATGCACATATATGTACATAAAGAGCT |
| | | complete cds | AGCTACCTGAGCTCTACCCAAGGTCGCGTTGATCG |
| | | | ATCGATCATGGCGCGGTTCGTGGACCCGCTGGTG |
| | | | GTGGGGGGGGTGATCGGCGAGGTGGTGGACCTGT |
| | | | TCGTGCCCTCCGTCTCCATGACCGTCGCCTATGGC |
| | | | CCCAAAGACATCAGCAACGGCTGCCTCCTCAAGC |
| | | | CGTCCGCCACCGCCGCGCCGCCGCTCGTCCGCATC |
| | | | TCCGGCCGCCGCGACGACCTCTACACGCTGATCAT |
| | | | GACGGACCCAGATGCGCCTAGCCCCAGCGACCCG |
| | | | ACCATGAGGGAGTACCTCCACTGGATAGTGACTA |
| | | | ACATACCAGGAGGAACGGATGCAAACAAAGGTG |
| | | | AGGAGGTGGTGGAGTACATGGGCCCGCGGCCGCC |
| | | | GGTCGGAATCCACCGCTACGTGCTGGTGCTGTTCG |
| | | | AGCAGAAGACGCGTGTGCACGCGGAGGGTCCCGG |
| | | | TGAGCGCGCCAACTTCAACACACGCGCGTTCGCG |
| | | | GCGGCGCACGAGCTCGGCCTCCCCACCGCCGTCG |
| | | | TGTACTTCAACGCGCAGAAAGAGCCGGCCAACCA |
| | | | CCGCCGCCGCTAGCTAGTAGCTCCAACAAGGGCG |
| | | | CGCCAGCTGAGCTGCGTGCGTGCAACCCACCACA |
| | | | CAGCCGCCGGCGAAGGCTGCCTATATGACCGGCG |
| | | | AATAAAAAGTCTTACTGCACCGTCCGTAAGCGTA |
| | | | CTCTCTGTTGGTATATGCTTGTCTTCAGGCTCTTGA |
| | | | GTCTATCTACTTAAATGTGGTTACCACTGAGTAAT |
| | | | AGAAGCAGTTGGCGCTTCGATCGATCATTCTAATA |
| | | | TCCGTACGTGTCAATCATTCCTGTTTCCATCATCTT |
| | | | GCATTTGAAGACGCATTGGTTCTACACCAAGGTGT |
| 47 | DNA | CmNACP: | GACTTTTTATTCAACAATCTCTCTCTCTCTCTCTCA |
| | | >FJ151402.1 | ACTTCCGATCAAGTCTCTCCGCCGTCTTTTCACCG |
| | | Cucurbita | GAGCTGACAATTCCGATCATTTTTTGCTTCCCTTA |
| | | maxima NAC- | AATTTCCGGCATGGAGGAACCACCGCCAAACGCC |
| | | domain | TTGGATTTGCCCCCTGGCTTCAGATTCCACCCCAC |
| | | containing | CGACGAGGAGATCGTCACTTATTACCTGATACATA |
| | | protein | AGATCACCGACGCCGCCTTCACTGCCACCGCCATC |
| | | (NACP1) | GGAGAAGCTGACCTGAATAAGTGTGAACCTTGGG |
| | | mRNA, | ATTTGCCACATAAAGCTAAGATGGGGGAAAAAGA |
| | | complete cds | ATGGTATTTTTTTTGCCAGAGAGACCGGAAATATC |
| | | | CGACCGGGATGAGAACGAACCGGGCGACTCAGAC |
| | | | CGGTTACTGGAAAGCGACCGGGAAAGACAAGGA |
| | | | GATTCTCAAGGGAAGAACGGTTCTGGCTGGTATG |
| | | | AAGAAAACGCTGGTTTTTTACAAAGGAAGAGCTC |
| | | | CCAAAGGTGAAAAGACCAATTGGGTCATGCATGA |
| | | | ATTTCGACTCGAACCCAAATTCTTTCAGTTTCTTG |
| | | | GTTTTCCCAAGCCCATTAAGGCTGATTGGGTTGTA |
| | | | TGTCGGGTTTTTCACAAGAACACAACGAACACGG |
| | | | TCGGAGTAGTGAAAAAGATTCAAACTTCTGATTTT |
| | | | TCTTCTTCTCTCCCACCTCTAATAGATCCCACAACT |
| | | | GCTCATACTCCAATCAGTGGCAGATTCGATAATGG |
| | | | TGAAGTCAACTGGAGGTTATCAGTACCATTCGATA |
| | | | ATTATGCAAATGATTACCATTATCATCGGCCTTTT |
| | | | TCAGCGACGAATACTGCAGTGACAATGATTTCGTC |
| | | | GTACCCATCGTCTGTCCCCGACGACGAATTCTTCT |
| | | | CATTTGATCAACTAGACGTCGGTGGAACAATGTC |
| | | | AATGGCGGCGGCGACGACAACAACAACAACAACT |
| | | | ATGGAGTGCAAAATAGAACAAGTTTCATGGTCAA |
| | | | CGATGAGCGGTGTGACACCGGAGATATCATCGTC |
| | | | GATTGACAACGAGGCAGCTCTCGAGTTCTGGGAC |
| | | | TACTGAAAATTGAAAGTAGATGTTATGATCGAAC |
| | | | AATGGCGATGCTTTGTTTTAAATGGGCATTTCCCA |
| | | | TATTGAACGTTTAAACAATGATTAATTGATTGCTA |
| | | | ATTATTATTATTTTTTTTTTTTGGTTACATAGTCCT |
| | | | TTTTGGGAAGGAATATTAGAACTTTCATGGGTTTG |
| | | | GTTTGTTGATTGTATTGATATGTAGCAATGTGACA |
| | | | TTGTATATAGCTTCTTTATCTTTTATTTTAACCGTT |
| | | | GCAAA |
| 48 | DNA | GAI: | TAATAATCATTTTTTTTCTTATAACCTTCCTCTCTA |
| | | >Y15193.1 | TTTTTACAATTTATTTTGTTATTA |
| | | Arabidopsis | GAAGTGGTAGTGGAGTGAAAAAACAAATCCTAAG |
| | | thaliana GAI | CAGTCCTAACCGATCCCCGAAGCTAAAGATTCTTC |
| | | gene | ACCTTCCCAAATAAAGCAAAACCTAGATCCGACA |
| | | | TTGAAGGAAAAACCTTTTAGATCCATCTCTGAAAA |
| | | | AAAACCAACCATGAAGAGAGATCATCATCATCAT |
| | | | CATCAAGATAAGAAGACTATGATGATGAATGAAG |
| | | | AAGACGACGGTAACGGCATGGATGAGCTTCTAGC |
| | | | TGTTCTTGGTTACAAGGTTAGGTCATCGGAAATGG |
| | | | CTGATGTTGCTCAGAAACTCGAGCAGCTTGAAGTT |
| | | | ATGATGTCTAATGTTCAAGAAGACGATCTTTCTCA |
| | | | ACTCGCTACTGAGACTGTTCACTATAATCCGGCGG |
| | | | AGCTTTACACGTGGCTTGATTCTATGCTCACCGAC |
| | | | CTTAATCCTCCGTCGTCTAACGCCGAGTACGATCT |
| | | | TAAAGCTATTCCCGGTGACGCGATTCTCAATCAGT |
| | | | TCGCTATCGATTCGGCTTCTTCGTCTAACCAAGGC |
| | | | GGCGGAGGAGATACGTATACTACAAACAAGCGGT |
| | | | TGAAATGCTCAAACGGCGTCGTGGAAACCACCAC |
| | | | AGCGACGGCTGAGTCAACTCGGCATGTTGTCCTG |
| | | | GTTGACTCGCAGGAGAACGGTGTGCGTCTCGTTCA |
| | | | CGCGCTTTTGGCTTGCGCTGAAGCTGTTCAGAAGG |
| | | | AGAATCTGACTGTGGCGGAAGCTCTGGTGAAGCA |
| | | | AATCGGATTCTTAGCTGTTTCTCAAATCGGAGCTA |
| | | | TGAGAAAAGTCGCTACTTACTTCGCCGAAGCTCTC |
| | | | GCGCGGCGGATTTACCGTCTCTCTCCGTCGCAGAG |
| | | | TCCAATCGACCACTCTCTCTCCGATACTCTTCAGA |
| | | | TGCACTTCTACGAGACTTGTCCTTATCTCAAGTTC |
| | | | GCTCACTTCACGGCGAATCAAGCGATTCTCGAAG |
| | | | CTTTTCAAGGGAAGAAAAGAGTTCATGTCATTGAT |
| | | | TTCTCTATGAGTCAAGGTCTTCAATGGCCGGCGCT |
| | | | TATGCAGGCTCTTGCGCTTCGACCTGGTGGTCCTC |
| | | | CTGTTTTCCGGTTAACCGGAATTGGTCCACCGGCA |
| | | | CCGGATAATTTCGATTATCTTCATGAAGTTGGGTG |
| | | | TAAGCTGGCTCATTTAGCTGAGGCGATTCACGTTG |
| | | | AGTTTGAGTACAGAGGATTTGTGGCTAACACTTTA |
| | | | GCTGATCTTGATGCTTCGATGCTTGAGCTTAGACC |
| | | | AAGTGAGATTGAATCTGTTGCGGTTAACTCTGTTT |
| | | | TCGAGCTTCACAAGCTCTTGGGACGACCTGGTGCG |
| | | | ATCGATAAGGTTCTTGGTGTGGTGAATCAGATTAA |
| | | | ACCGGAGATTTTCACTGTGGTTGAGCAGGAATCG |
| | | | AACCATAATAGTCCGATTTTCTTAGATCGGTTTAC |
| | | | TGAGTCGTTGCATTATTACTCGACGTTGTTTGACT |
| | | | CGTTGGAAGGTGTACCGAGTGGTCAAGACAAGGT |
| | | | CATGTCGGAGGTTTACTTGGGTAAACAGATCTGCA |
| | | | ACGTTGTGGCTTGTGATGGACCTGACCGAGTTGAG |
| | | | CGTCATGAAACGTTGAGTCAGTGGAGGAACCGGT |
| | | | TCGGGTCTGCTGGGTTTGCGGCTGCACATATTGGT |
| | | | TCGAATGCGTTTAAGCAAGCGAGTATGCTTTTGGC |
| | | | TCTGTTCAACGGCGGTGAGGGTTATCGGGTGGAG |
| | | | GAGAGTGACGGCTGTCTCATGTTGGGTTGGCACA |
| | | | CACGACCGCTCATAGCCACCTCGGCTTGGAAACTC |
| | | | TCCACCAATTAGATGGTGGCTCAATGAATTGATCT |
| | | | GTTGAACCGGTTATGATGATAGATTTCCGACCGAA |
| | | | GCCAAACTAAATCCTACTGTTTTTCCCTTTGTCACT |
| | | | TGTTAAGATCTTATCTTTCATTATATTAGGTAATTG |
| | | | AAAAATTTCTAAATTACTCACACTGGC |
| 49 | DNA | LeT6 a tomato | AAAGAAAAAAGGAATATTGTGTGTTTGCTTTTTTT |
| | | KNOX gene: | TCTGACTAGTAGTATTGCTAACTATGTATTCCATT |
| | | >AF000141.1 | AAGGATTTGCTGTGAAAAAGCCTGATATCAGTAA |
| | | Lycopersicon | GCATAAAACTCGGGAGATCACTTACACACACACA |
| | | esculentum | CACCCTCCTAAAAAAGAGAAGAGAGATTTACTGT |
| | | class I knotted- | TAAACAGAGGTTTTTTTCCATTTCTTTTTTTTTTTC |
| | | like | AGTGTGTGTGTGAGAGAAAGAGATGATTTTCATA |
| | | homeodomain | GGCACAAACAAATAGAAAGGAACAAAATTTAGA |
| | | protein (LeT6) | GTGAAGAAGAAAGTGTGTGAGAGAATAATGGAG |
| | | mRNA, | GGTGGTTCTAGTGGAAATACTAGTACATCTTGTTT |
| | | complete cds | AATGATGATGGGATATGGAGATCATGAAAACAAC |
| | | | AACAACAACAATGGAAATGGTAATGGAAATGGAA |
| | | | ATGGAAATGTAACAATTTGTGCTCCTCCAATGATG |
| | | | ATGATGATGCCTCCTCCTCCTCCTTCTTTAACTAAC |
| | | | AATAACAATGCAGAAACAAGCAACAACAACATCC |
| | | | TTTTTCTTCCTTTCATGGACAACAACAACAACAAT |
| | | | AATCCTCAAGAAGACAACAACTCTTCTTCTTCTTC |
| | | | CATCAAGTCAAAGATTATGGCTCATCCTCACTACC |
| | | | ATCGTCTCTTGACTGCTTATCTCAATTGTCAAAAG |
| | | | ATAGGAGCTCCGCCAGAAGTGGTGGCAAGGCTAG |
| | | | AGGAAATATGTGCCACGTCAGCAACAATGGGCCG |
| | | | TAGCAGTAGTAGTAGTGGTGGTGGAATCATTGGA |
| | | | GAAGATCCTGCACTAGATCAGTTCATGGAGGCTT |
| | | | ATTGTGAGATGCTGACAAAATATGAACAAGAACT |
| | | | CTCAAAACCCTTCAAGGAAGCCATGGTTTTTCTTT |
| | | | CAAGAATTGAGTGTCAGTTCAAAGCTTTAACTCTT |
| | | | GCACCTAATTCTTCTCATGAATCTGCTTTGGGCGA |
| | | | GGCAATGGATAGAAATGGATCATCTGATGAAGAG |
| | | | GTTGACGTGAATAACAGTTTCATCGACCCCCAGGC |
| | | | TGAGGATAGAGAGCTCAAAGGTCAATTGTTGCGT |
| | | | AAGTACAGCGGTTACTTGGGAAGCCTTAAGCAGG |
| | | | AGTTCATGAAGAAGAGGAAGAAAGGCAAGCTGCC |
| | | | TAAGGAAGCAAGGCAACAATTGGTGGATTGGTGG |
| | | | CTTAGACATATTAAATGGCCATATCCATCGGAATC |
| | | | TCAGAAGCTTGCACTAGCTGAATCAACGGGATTG |
| | | | GACCAGAAGCAAATAAACAACTGGTTTATCAATC |
| | | | AAAGAAAGAGGCATTGGAAACCATCAGAAGATAT |
| | | | GCAGTTTGTTGTGATGGATGCTGCTCATCCACATT |
| | | | ACTATATGGATAATGTTCTTGCTAACCATTTCCCA |
| | | | ATGGATATGACACCCTCTCTCCTCTGAATTAAGAT |
| | | | TTGTCATTATTAATATCAAGGATGTTTAATTAATT |
| | | | TGCATATTACTTGTGTGCATGTAGTAGTACAAGCT |
| | | | ATTGTGACACAATCAACTTTTTATTAGACCAAATA |
| | | | TATAAAGTGCTTGTAATAGATCTTTCTATTATCAT |
| | | | CTTTAATTATGGAATTAAATAGTTTGTACTTGCTA |
| | | | AAA |
| 50 | DNA | BEL5: | CATGCAGAGATAAAAATATAGATCAGTCTGACAA |
| | | >NM_001287992.1 | GAAGGCAACTTCTCAAAGCTTAGAGAGCTACCAC |
| | | Solanum | CCGAAGATAGACAGTTAGTTACATGTACTGTTATA |
| | | tuberosum | GATAAAAGGAGAAATCCGAAGAAGAAAGAATTTT |
| | | BEL1-related | TTTTGCAGATATGTACTATCAAGGAACCTCGGATA |
| | | homeotic | ATACTAATATACAAGCTGATCATCAACAACGTCAT |
| | | protein 5 | AATCATGGGAATAGTAATAATAATAATATTCAGA |
| | | (BEL5), mRNA | CACTTTATTTGATGAACCCTAACAATTATATGCAA |
| | | | GGCTACACTACTTCTGACACACAGCAGCAGCAGC |
| | | | AGTTACTTTTCCTGAATTCTTCACCAGCAGCAAGC |
| | | | AACGCGCTTTGCCATGCGAATATACAACACGCGC |
| | | | CGCTGCAACAGCAGCACTTTGTCGGTGTGCCTCTT |
| | | | CCGGCAGTAAGTTTGCACGATCAGATCAATCATC |
| | | | ATGGACTTTTACAGCGCATGTGGAACAACCAAGA |
| | | | TCAATCTCAGCAGGTGATAGTACCATCGTCGACG |
| | | | GGGGTTTCTGCCACGTCATGTGGCGGGATCACCAC |
| | | | GGACTTGGCGTCTCAATTGGCGTTTCAGAGGCCGA |
| | | | TTCCGACACCACAACACCGACAGCAGCAACAACA |
| | | | GCAAGGCGGTCTATCTCTAAGCCTTTCTCCTCAGC |
| | | | TACAACAGCAAATTAGTTTCAATAACAATATTTCA |
| | | | TCCTCATCACCAAGGACAAATAATGTTACTATTAG |
| | | | GGGAACATTAGATGGAAGTTCTAGCAACATGGTT |
| | | | TTAGGCTCTAAGTATCTGAAAGCTGCACAAGAGC |
| | | | TTCTTGATGAAGTTGTTAATATTGTTGGAAAAAGC |
| | | | ATCAAAGGAGATGATCAAAAGAAGGATAATTCAA |
| | | | TGAATAAAGAATCAATGCCTTTGGCTAGTGATGTC |
| | | | AACACTAATAGTTCTGGTGGTGGTGAAAGTAGCA |
| | | | GCAGGCAGAAAAATGAAGTTGCTGTTGAGCTTAC |
| | | | AACTGCTCAAAGACAAGAACTTCAAATGAAAAAA |
| | | | GCCAAGCTTCTTGCCATGCTTGAAGAGGTGGAGC |
| | | | AAAGGTACAGACAGTACCATCACCAAATGCAAAT |
| | | | AATTGTATTATCATTTGAGCAAGTAGCAGGAATTG |
| | | | GATCAGCCAAATCATACACTCAATTAGCTTTGCAT |
| | | | GCAATTTCGAAGCAATTCAGATGCCTAAAGGATG |
| | | | CAATTGCTGAGCAAGTAAAGGCGACGAGCAAGAG |
| | | | TTTAGGTGAAGAGGAAGGCTTGGGAGGGAAAATC |
| | | | GAAGGCTCAAGACTCAAATTTGTGGACCATCATCT |
| | | | AAGGCAACAACGCGCGCTGCAACAGATAGGAATG |
| | | | ATGCAACCAAATGCTTGGAGACCCCAAAGAGGTT |
| | | | TACCTGAAAGAGCTGTCTCTGTCCTTCGTGCTTGG |
| | | | CTTTTCGAGCATTTTCTTCATCCTTACCCAAAGGA |
| | | | TTCAGACAAAATCATGCTTGCTAAGCAAACGGGG |
| | | | CTAACAAGGAGCCAGGTGTCTAACTGGTTCATAA |
| | | | ATGCTCGAGTTCGATTATGGAAGCCAATGGTAGA |
| | | | AGAAATGTACTTGGAAGAAGTGAAGAATCAAGAA |
| | | | CAAAACAGTACTAATACTTCAGGAGATAACAAAA |
| | | | ACAAAGAGACCAATATAAGTGCTCCAAATGAAGA |
| | | | GAAACATCCAATTATTACTAGCAGCTTATTACAAG |
| | | | ATGGTATTACTACTACTCAAGCAGAAATTTCTACC |
| | | | TCAACTATTTCAACTTCCCCTACTGCAGGTGCTTC |
| | | | ACTTCATCATGCTCACAATTTCTCCTTCCTTGGTTC |
| | | | ATTCAACATGGATAATACTACTACTACTGTTGATC |
| | | | ATATTGAAAACAACGCGAAAAAGCAAAGAAATG |
| | | | ACATGCACAAGTTTTCTCCAAGTAGTATTCTTTCA |
| | | | TCTGTTGACATGGAAGCCAAAGCTAGAGAATCAT |
| | | | CAAATAAAGGGTTTACTAATCCTTTAATGGCAGCA |
| | | | TACGCGATGGGAGATTTTGGAAGGTTTGATCCTCA |
| | | | TGATCAACAAATGACCGCGAATTTTCATGGAAAT |
| | | | AATGGTGTCTCTCTTACTTTAGGACTTCCTCCTTCT |
| | | | GAAAACCTAGCCATGCCAGTGAGCCAACAAAATT |
| | | | ACCTTTCTAATGACTTGGGAAGTAGGTCTGAAATG |
| | | | GGGAGTCATTACAATAGAATGGGATATGAAAACA |
| | | | TTGATTTTCAGAGTGGGAATAAGCGATTTCCGACT |
| | | | CAACTATTACCAGATTTTGTTACAGGTAATCTAGG |
| | | | AACATGAATACCAGAAAGTCTCGTATTGATAGCT |
| | | | GAAAAGATAAAAGGAAGTTAGGGATACTCTTATA |
| | | | TTGTGTGAGGCCTTCTGGCCCAAGTCGGAGGACCC |
| | | | AATTTGATACAACCTATCATAGGAGAAAAGAAGT |
| | | | GGAGACTAAATTAAAGTAACAAAATTTTAAAGCA |
| | | | CACTTTCTAGTATATATACTTCTTTTTTTTATAGTA |
| | | | TAGAAAAGAAGAGATTTTGTGCTTTAGTGTATAG |
| | | | ATAGAGTCTACTTAGTATAGGTTATACTTCTAGTT |
| | | | CCTTGAGAAGATTGATACAACTAGTAGTATTTTTT |
| | | | TTCTTTTGGGTTGGCTTGGAGTACTATTTTAAGTTA |
| | | | TTGGAAACTAGCTATAGTAAATGTTGTAAAGTTGT |
| | | | GATATTGTTCCTCTCAATTTGCATATAATTTGAAA |
| | | | TATTTTGTACCTACTAGCTAGTCTCTAAATTATGTT |
| | | | TCCATTGCTTGTAATTGCAATTTTATTTGAATTTTG |
| | | | TGCTATCATTATTAGATTAGCAAAAAAAAAAAAA |
| | | | AAAAA |
| 51 | DNA | AT5G57885.1 | ATCAGAGTGGCGCAGCGGAAGCGTGGTGGGCCCA |
| | | (tRNA-Met) | TAACCCACAGGTCCCAGGATCGAAACCTGGCTCT |
| | | | GATA |
| 52 | DNA | AT1G71700 | GCACCAGTGGTCTAGTGGCATGATAGTACCCTGCC |
| | | (tRNA-Gly) | ACGGTACAGACCCGGGTTCAATTCCCGGCTGGTG |
| | | | CA |
| 53 | PRO | FnCas12a | ISEDLLQNYSDVYFKLKKSDDDNLQKDFKSAKDTIK |
| | | (UniProtKB/ | KQISEYIKDSEKFKNLFNQNLIDAKKGQESDLILWLK |
| | | Swiss-Prot: | QSKDNGIELFKANSDITDIDEALEIIKSFKGWTTYFKG |
| | | A0Q7Q2.1); | FHENRKNVYSSNDIPTSIIYRIVDDNLPKFLENKAKY |
| | | US20160208243; | ESLKDKAPEAINYEQIKKDLAEELTFDIDYKTSEVNQ |
| | | and WO | RVFSLDEVFEIANFNNYLNQSGITKFNTIIGGKFVNG |
| | | 2017/189308) | ENTKRKGINEYINLYSQQINDKTLKKYKMSVLFKQI |
| | | | LSDTESKSFVIDKLEDDSDVVTTMQSFYEQIAAFKTV |
| | | | EEKSIKETLSLLFDDLKAQKLDLSKIYFKNDKSLTDL |
| | | | SQQVEDDYSVIGTAVLEYITQQIAPKNLDNPSKKEQE |
| | | | LIAKKTEKAKYLSLETIKLALEEFNKHRDIDKQCRFE |
| | | | EILANFAAIPMIFDEIAQNKDNLAQISIKYQNQGKKD |
| | | | LLQASAEDDVKAIKDLLDQTNNLLHKLKIFHISQSED |
| | | | KANILDKDEHFYLVFEECYFELANIVPLYNKIRNYIT |
| | | | QKPYSDEKFKLNFENSTLANGWDKNKEPDNTAILFI |
| | | | KDDKYYLGVMNKKNNKIFDDKAIKENKGEGYKKIV |
| | | | YKLLPGANKMLPKVFFSAKSIKFYNPSEDILRIRNHS |
| | | | THTKNGSPQKGYEKFEFNIEDCRKFIDFYKQSISKHP |
| | | | EWKDFGFRFSDTQRYNSIDEFYREVENQGYKLTFEN |
| | | | ISESYIDSVVNQGKLYLFQIYNKDFSAYSKGRPNLHT |
| | | | LYWKALFDERNLQDVVYKLNGEAELFYRKQSIPKKI |
| | | | THPAKEAIANKNKDNPKKESVFEYDLIKDKRFTEDK |
| | | | FFFHCPITINFKSSGANKFNDEINLLLKEKANDVHILS |
| | | | IDRGERHLAYYTLVDGKGNIIKQDTFNIIGNDRMKT |
| | | | NYHDKLAAIEKDRDSARKDWKKINNIKEMKEGYLS |
| | | | QVVHEIAKLVIEYNAIVVFEDLNFGFKRGRFKVEKQ |
| | | | VYQKLEKMLIEKLNYLVFKDNEFDKTGGVLRAYQL |
| | | | TAPFETFKKMGKQTGIIYYVPAGFTSKICPVTGFVNQ |
| | | | LYPKYESVSKSQEFFSKFDKICYNLDKGYFEFSFDYK |
| | | | NFGDKAAKGKWTIASFGSRLINFRNSDKNHNWDTR |
| | | | EVYPTKELEKLLKDYSIEYGHGECIKAAICGESDKKF |
| | | | FAKLTSVLNTILQMRNSKTGTELDYLISPVADVNGN |
| | | | FFDSRQAPKNMPQDADANGAYHIGLKGLMLLGRIK |
| | | | NNQEGKKLNLVIKNEEYFEFVQNRNN |
| 54 | RNA | FnCas12a direct | GGGUCUAAGAACUUUAAAUAAUUUCUACUGUUG |
| | | repeat (Fonfara | UACAU |
| | | et al. | |
| | | Nature 2016, | |
| | | 532: 517-521. | |
| | | US2016- | |
| | | 0208243; WO | |
| | | 2017/189308) | |
| 55 | PRO | LbCpf1 (from | AASKLEKFTNCYSLSKTLRFKAIPVGKTQENIDNKRL |
| | | Lachnospiraceae | LVEDEKRAEDYKGVKKLLDRYYLSFINDVLHSIKLK |
| | | bacterium | NLNNYISLFRKKTRTEKENKELENLEINLRKEIAKAF |
| | | ND2006; | KGAAGYKSLFKKDIIETILPEAADDKDEIALVNSENG |
| | | UniProtKB: | FTTAFTGFFDNRENMFSEEAKSTSIAFRCINENLTRYI |
| | | A0A182DWE3) | SNMDIFEKVDAIFDKHEVQEIKEKILNSDYDVEDFFE |
| | | | GEFFNFVLTQEGIDVYNAIIGGFVTESGEKIKGLNEYI |
| | | | NLYNAKTKQALPKFKPLYKQVLSDRESLSFYGEGYT |
| | | | SDEEVLEVFRNTLNKNSEIFSSIKKLEKLFKNFDEYSS |
| | | | AGIFVKNGPAISTISKDIFGEWNLIRDKWNAEYDDIH |
| | | | LKKKAVVTEKYEDDRRKSFKKIGSFSLEQLQEYADA |
| | | | DLSVVEKLKEIIIQKVDEIYKVYGSSEKLFDADFVLE |
| | | | KSLKKNDAVVAIMKDLLDSVKSFENYIKAFFGEGKE |
| | | | TNRDESFYGDFVLAYDILLKVDHIYDAIRNYVTQKP |
| | | | YSKDKFKLYFQNPQFMGGWDKDKETDYRATILRYG |
| | | | SKYYLAIMDKKYAKCLQKIDKDDVNGNYEKINYKL |
| | | | LPGPNKMLPKVFFSKKWMAYYNPSEDIQKIYKNGTF |
| | | | KKGDMFNLNDCHKLIDFFKDSISRYPKWSNAYDFNF |
| | | | SETEKYKDIAGFYREVEEQGYKVSFESASKKEVDKL |
| | | | VEEGKLYMFQIYNKDFSDKSHGTPNLHTMYFKLLF |
| | | | DENNHGQIRLSGGAELFMRRASLKKEELVVHPANSP |
| | | | IANKNPDNPKKTTTLSYDVYKDKRFSEDQYELHIPIA |
| | | | INKCPKNIFKINTEVRVLLKHDDNPYVIGIDRGERNL |
| | | | LYIVVVDGKGNIVEQYSLNEIINNFNGIRIKTDYHSLL |
| | | | DKKEKERFEARQNWTSIENIKELKAGYISQVVHKICE |
| | | | LVEKYDAVIALEDLNSGFKNSRVKVEKQVYQKFEK |
| | | | MLIDKLNYMVDKKSNPCATGGALKGYQITNKFESF |
| | | | KSMSTQNGFIFYIPAWLTSKIDPSTGFVNLLKTKYTSI |
| | | | ADSKKFISSFDRIMYVPEEDLFEFALDYKNFSRTDAD |
| | | | YIKKWKLYSYGNRIRIFAAAKKNNVFAWEEVCLTS |
| | | | AYKELFNKYGINYQQGDIRALLCEQSDKAFYSSFMA |
| | | | LMSLMLQMRNSITGRTDVDFLISPVKNSDGIFYDSR |
| | | | NYEAQENAILPKNADANGAYNIARKVLWAIGQFKK |
| | | | AEDEKLDKVKIAISNKEWLEYAQTSVK |
| 56 | RNA | LbCpf1 DR | GUUUCAAAGAUUAAAUAAUUUCUACUAAGUGUA |
| | | (from | GAU |
| | | Lachnospiraceae | |
| | | bacterium | |
| | | ND2006; | |
| | | Zetsche et al., | |
| | | doi.org/10.1101/ | |
| | | 134015) | |
| 57 | PRO | Cas12j-1protein | MADTPTLFTQFLRHHLPGQRFRKDILKQAGRILANK |
| | | (Pausch et al., | GEDATIAFLRGKSEESPPDFQPPVKCPIIACSRPLTEW |
| | | 2020 Science 17 | PIYQASVAIQGYVYGQSLAEFEASDPGCSKDGLLGW |
| | | July 2020: Vol. | FDKTGVCTDYFSVQGLNLIFQNARKRYIGVQTKVTN |
| | | 369, Issue 6501, | RNEKRHKKLKRINAKRIAEGLPELTSDEPESALDETG |
| | | pp. 333-337) | HLIDPPGLNTNIYCYQQVSPKPLALSEVNQLPTAYAG |
| | | | YSTSGDDPIQPMVTKDRLSISKGQPGYIPEHQRALLS |
| | | | QKKHRRMRGYGLKARALLVIVRIQDDWAVIDLRSL |
| | | | LRNAYWRRIVQTKEPSTITKLLKLVTGDPVLDATRM |
| | | | VATFTYKPGIVQVRSAKCLKNKQGSKLFSERYLNET |
| | | | VSVTSIDLGSNNLVAVATYRLVNGNTPELLQRFTLPS |
| | | | HLVKDFERYKQAHDTLEDSIQKTAVASLPQGQQTEI |
| | | | RMWSMYGFREAQERVCQELGLADGSIPWNVMTAT |
| | | | STILTDLFLARGGDPKKCMFTSEPKKKKNSKQVLYK |
| | | | IRDRAWAKMYRTLLSKETREAWNKALWGLKRGSP |
| | | | DYARLSKRKEELARRCVNYTISTAEKRAQCGRTIVA |
| | | | LEDLNIGFFHGRGKQEPGWVGLFTRKKENRWLMQA |
| | | | LHKAFLELAHHRGYHVIEVNPAYTSQTCPVCRHCDP |
| | | | DNRDQHNREAFHCIGCGFRGNADLDVATHNIAMVA |
| | | | ITGESLKRARGSVASKTPQPLAAE |
| 58 | RNA | Cas12j-2 DR | GGAGAGAUCUCAAACGAUUGCUCGAUUAGUCGA |
| | | sequence | GAC |
| | | (Pausch et al. | |
| | | Science 2020, | |
| | | 369(6501): | |
| | | 333-337) | |
| 59 | PRO | Cas12j-2 | MPKPAVESEFSKVLKKHFPGERFRSSYMKRGGKILA |
| | | protein (Pausch | AQGEEAVVAYLQGKSEEEPPNFQP |
| | | et al. | PAKCHVVTKSRDFAEWPIMKASEAIQRYIYALSTTE |
| | | Science 2020, | RAACKPGKSSESHAAWFAATGVSNHGYSHVQGLNL |
| | | 369(6501): | IFDHTLGRYDGVLKKVQLRNEKARARLESINASRAD |
| | | 333-337) | EGLPEIKAEEEEVATNETGHLLQPPGINPSFYVYQTIS |
| | | | PQAYRPRDEIVLPPEYAGYVRDPNAPIPLGVVRNRC |
| | | | DIQKGCPGYIPEWQREAGTAISPKTGKAVTVPGLSPK |
| | | | KNKRMRRYWRSEKEKAQDALLVTVRIGTDWVVID |
| | | | VRGLLRNARWRTIAPKDISLNALLDLFTGDPVIDVR |
| | | | RNIVTFTYTLDACGTYARKWTLKGKQTKATLDKLT |
| | | | ATQTVALVAIDLGQTNPISAGISRVTQENGALQCEPL |
| | | | DRFTLPDDLLKDISAYRIAWDRNEEELRARSVEALPE |
| | | | AQQAEVRALDGVSKETARTQLCADFGLDPKRLPWD |
| | | | KMSSNTTFISEALLSNSVSRDQVFFTPAPKKGAKKK |
| | | | APVEVMRKDRTWARAYKPRLSVEAQKLKNEALWA |
| | | | LKRTSPEYLKLSRRKEELCRRSINYVIEKTRRRTQCQI |
| | | | VIPVIEDLNVRFFHGSGKRLPGWDNFFTAKKENRWF |
| | | | IQGLHKAFSDLRTHRSFYVFEVRPERTSITCPKCGHC |
| | | | EVGNRDGEAFQCLSCGKTCNADLDVATHNLTQVAL |
| | | | TGKTMPKREEPRDAQGTAPARKTKKASKSKAPPAE |
| | | | REDQTPAQEPSQTS |
| 60 | RNA | Cas12j-2 DR | GUCGGAACGCUCAACGAUUGCCCCUCACGAGGG |
| | | sequence | GAC |
| | | (Pausch et al. | |
| | | Science 2020, | |
| | | 369(6501): | |
| | | 333-337) | |
| 61 | PRO | Cas12j-3 | MEKEITELTKIRREFPNKKFSSTDMKKAGKLLKAEG |
| | | protein (Pausch | PDAVRDFLNSCQEIIGDFKPPVKTNIVSISRPFEEWPV |
| | | et al. Science | SMVGRAIQEYYFSLTKEELESVHPGTSSEDHKSFFNI |
| | | 2020, | TGLSNYNYTSVQGLNLIFKNAKAIYDGTLVKANNK |
| | | 369(6501): | NKKLEKKFNEINHKRSLEGLPIITPDFEEPFDENGHLN |
| | | 333-337) | NPPGINRNIYGYQGCAAKVFVPSKHKMVSLPKEYEG |
| | | | YNRDPNLSLAGFRNRLEIPEGEPGHVPWFQRMDIPE |
| | | | GQIGHVNKIQRFNFVHGKNSGKVKFSDKTGRVKRY |
| | | | HHSKYKDATKPYKFLEESKKVSALDSILAIITIGDDW |
| | | | VVFDIRGLYRNVFYRELAQKGLTAVQLLDLFTGDPV |
| | | | IDPKKGVVTFSYKEGVVPVFSQKIVPRFKSRDTLEKL |
| | | | TSQGPVALLSVDLGQNEPVAARVCSLKNINDKITLD |
| | | | NSCRISFLDDYKKQIKDYRDSLDELEIKIRLEAINSLE |
| | | | TNQQVEIRDLDVFSADRAKANTVDMFDIDPNLISWD |
| | | | SMSDARVSTQISDLYLKNGGDESRVYFEINNKRIKRS |
| | | | DYNISQLVRPKLSDSTRKNLNDSIWKLKRTSEEYLK |
| | | | LSKRKLELSRAVVNYTIRQSKLLSGINDIVIILEDLDV |
| | | | KKKFNGRGIRDIGWDNFFSSRKENRWFIPAFHKAFS |
| | | | ELSSNRGLCVIEVNPAWTSATCPDCGFCSKENRDGI |
| | | | NFTCRKCGVSYHADIDVATLNIARVAVLGKPMSGP |
| | | | ADRERLGDTKKPRVARSRKTMKRKDISNSTVEAMV |
| | | | TA |
| 62 | RNA | Cas12j-3 DR | ACCAAAACGACUAUUGAUUGCCCAGUACGCUGG |
| | | sequence | GAC |
| | | (Pausch et al. | |
| | | Science 2020, | |
| | | 369(6501): | |
| | | 333-337) | |

The meristem transport-competence (MTC) potential can be determined for any variants, fragments, and/or orthologs of the aforementioned FT, GAI, CmNACP, LeT6 a tomato KNOX gene, BEL5, or tRNA-like RNAs. A side-by-side comparison with a known MTS as a positive control is useful. As such, a number of configurations can be used. One approach is to fuse candidate sequences to guide sequences of characterized editing potential for a species of interest. RNA sequences can be introduced into the phloem of an individual plant that expresses or translates at least in the meristem a nuclease capable of associating with the guide sequence and producing the intended genomic alteration. The RNA sequences can be expressed in vitro and introduced into the phloem as purified molecules. For example, a concentrated solution of RNA molecules of interest can be applied to a mechanically injured plant tissue, such as a cut or abraded leaf, stem, or meristem dome. RNAs can be coated on particles, such as micro or nano-scale particles such as gold or tungsten, for biolistic delivery. Alternatively, the RNA sequences could be incorporated into RNA viruses introduced in the plants (Jackson et al. Front. Plant Sci. 2012, 3:127; Ali et al. Mol. Plant 2015, 8:1288-1291; Cody et al. Plant Physiol. 2017, 175:23-35; Ali et al. Virus Res. 2018, 244:333-337; Gao et al. New Phytol. 2019, 223:2120-2133) or the MTC can be assayed by introducing RNAs by grafting, i.e. the RNA molecules can be expressed in the rootstock of a grafted plant, and their effect observed in the scion (Zhang et al. Plant Cell, 2016, 28:1237-1249; Huang et al. Plant Physiol. 2018, 178:783-794). MTS candidates can be assayed for longer and/or more complex RNA molecules, or mixtures of RNA molecules, that comprise not only guide or processable guide regions, but also nuclease-encoding sequences. A clear readout of MTC is detection of the expected genomic alterations in progeny plants, which can be done by sequencing of the target genomic region, or even by whole genome sequencing. But alternative readouts can be designed that may be more convenient in some cases. For example, the guide sequences may be directed to disrupt or repair a reporter gene, such as a transgene encoding a fluorescent polypeptide. The expected genetic changes can then be evaluated in the treated plants by measuring changes in the reporter. Another convenient genomic alteration target in many species is phytoene desaturase (PDS), with the albino phenotype of the mutant serving as a readout.

In some embodiments, the meristem transport segment (MTS) comprises a Flowering Locus T (FT)-derived sequence, a tRNA like sequence (TLS), a meristem transport component (MTC); or an RNA hairpin comprising a first stem of 8 to 12 nucleotides, at least one variable bulge, a second stem of 4 to 7 nucleotides, and a variable loop. In some embodiments, the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24. In some embodiments, the TLS comprises the nucleotide sequence set forth in SEQ ID NO: 29 or 30.

In some embodiments, the nucleic acid encoding the MTS is located 3′ of the nucleic acid encoding the Cas nuclease and/or 3′ of the nucleic acid encoding the guide RNA. In some embodiments, the nucleic acid encoding the MTS is located 5′ of the nucleic acid encoding the Cas nuclease and/or 5′ of the nucleic acid encoding the guide RNA.

In some embodiments, the plant further comprises nucleic acid encoding a detectable marker fused to a nucleic acid encoding an MTS.

### J. Genome Modifications

The reagents and methods described provide a relatively easy and convenient solution for producing plants, plant parts, plant tissues, and plant cells with altered genomes, i.e., individuals and/or individual cells with designed DNA sequence modifications (e.g. Indels or epigenetic alterations). The methods provided herein can be applied to edit one or more genomic regions selected independently from the group consisting of a gene, an array of tandemly duplicated genes, a multigene family, an enhancer, a suppressor, a promoter, a termination sequence, a splice acceptor sequence, a splice donor sequence, an intron, an exon, an siRNA, a sequence encoding a non-coding RNA, a microRNA, a transgene, and a quantitative trait locus (QTL). In some embodiments, the edit results in the insertion or deletion of nucleotides at or near the target sequence. In some embodiments, the edit results in an insertion of at least 1, 2, 3, 4, 5, 6, 7, 8, 9, 10, 15, 20, 25, 30, 35, or 40 nucleotides at or near the target sequence. In some embodiments, the edit results in a deletion of at least 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 12500, 15000, 17500, 20000, 22500, or 25000 nucleotides at or near the target sequence. In some embodiments, the edit results in a nucleotide substitution at or near the target sequence. In some embodiments, the edit results in a substitution of at least 1, 2, 3, 4, 5, 6, 7, 8, 9, 10, 15, 20, 25, 30, 35, 40, 45, 50, 60, 70, 80, 90, or 100 nucleotides at or near the target sequence. In most embodiments, the methods and systems rely on DNA or RNA molecules produced with established molecular biology techniques. The DNA or RNA molecules, which comprise genome-editing reagents, are then introduced into a plant and taken up into meristematic cells. The meristematic cell genomes are thus altered, and the DNA sequence modifications (e.g. Indels or epigenetic alterations) are carried into germline cells and subsequent generations.

Very often, mutated seeds from plants edited with the reagents and methods described here are collected for phenotypic characterization. In some cases, pollen from edited plants is used in crosses with other individuals, or mutated individuals are pollinated with pollen of unedited plants or wildtype plants.

The embodiments described methods and reagents can have many advantages over other known solutions. The techniques presented generally bypass callus induction or tissue culture that are necessary for alternative or widely practiced genome editing procedures, thus speeding up (i.e., accelerating) and lowering or reducing the cost of the process of producing plants with targeted DNA sequence modifications. Epigenetic resetting (i.e., interference) is also eliminated. The editing can be performed in individuals of an elite genetic background, making lengthy backcrossing schemes unnecessary.

Plants comprising the RNA molecules that comprise a Cas nuclease and/or guide RNA(s) that are operably linked to MTS sequences are also provided herein. In certain embodiments, such RNA molecules will be present at detectable concentrations in the plants for only a certain period of time following a stimulus. For example, the concentrations of RNA molecules comprising guide RNAs separated by processing elements comprising direct repeats (DR, i.e., pre-crRNAs comprising a full-length direct repeat (full-DR-crRNA)) which are capable of being processed (i.e., cleaved) by an RNA-guided nuclease are expected to decrease over time when the RNA-guided nuclease is also present in the plant. The concentrations of RNA molecules comprising guide RNAs separated by processing elements comprising direct repeats which are capable of being processed by an RNA-guided nuclease are also expected to be decreased in tissues where the RNA-guided nuclease is located. Nonetheless, the unprocessed RNA molecules can be detected by a variety of techniques that include reverse transcription polymerase chain reaction (RT-PCR) assays where oligonucleotide primers and optionally detection probes which specifically amplify and detect the unprocessed RNA molecule comprising the Cas nuclease and/or guide RNA(s) that are operably linked to MTS sequences are used. Such plants can comprise any of the RNA molecules or combinations of RNA molecules present in the compositions provided herein that are used to contact the plants. In certain embodiments, an active form of the RNA guided nuclease is predominantly localized in meristem tissue of the plant. In certain embodiments, the RNA-guided nuclease can be encoded by an RNA molecule that optionally further comprises an operably linked MTS sequence. In certain embodiments, the RNA-guided nuclease can be encoded by DNA that is operably linked to promoters that include a root-preferred or root-specific promoter which is active in root cells. In certain embodiments, the RNA-guided nuclease can be encoded by DNA that is operably linked to constitutively active promoters. DNA encoding the RNA-guided nuclease can be provided in a transgene that is stably integrated in the genome of the plant, in DNA that is not integrated into the plant genome, or in DNA provided in a viral vector (e.g., a geminivirus replicon). Geminivirus DNA replicons suitable for delivery of DNA molecules encoding an RNA-guided nuclease to plants include a Beet Yellow Dwarf Virus replicon (Baltes et al. Plant Cell 2014, 26(1): 151-63; doi: 10.1105/tpc.113.119792).

In certain embodiments, an MTS is operably linked to a CRISPR Cas system comprising a plurality of guide RNAs (e.g., 2, 3, 4, or more guide RNAs) separated by processing elements to provide for gene editing at a plurality of genomic locations targeted by each guide RNA. In certain embodiments, the plurality of guide RNAs are separated by processing elements comprising direct repeats (DR; i.e., pre-crRNAs comprising a full-length direct repeat (full-DR-crRNA)) which are capable of being processed (i.e., cleaved) by an RNA-guided nuclease. Examples of such DRs include the Cas12a DR (e.g., SEQ ID NO: 54 or 56) which can be cleaved by a Cas12a guided nuclease (e.g., SEQ ID NO: 53 or 55, respectively). Cleavage of RNAs comprising Cas12a DRs by Cas12a has been described (Fonfara et al. Nature 2016, 532:517-521, doi.org/10.1038/nature17945); US20160208243; WO 2017/189308). Other examples of such DRs include the Cas12j DRs (e.g., SEQ ID NO: 58, 60, or 62) which can be cleaved by a Cas12j guided nuclease ((e.g., SEQ ID NO: 57, 59, or 61, respectively). In such embodiments, the crRNA portion of the DR can remain as a part of the gRNA after processing and can be recognized by the RNA guided nuclease to provide for editing of genomic DNA recognized via hybridization of the gRNA to the targeted genomic site.

In some embodiments, the meristem is part of a plant scion grafted onto a rootstock. In other embodiments, the meristem is part of a non-grafted plant.

### II. Targets of Genomic Modification

Embodiments of the polynucleotides, compositions, engineered systems, and methods disclosed herein are useful in editing or effecting a sequence-specific modification of a target DNA sequence or target gene in a DNA molecule, a chromosome, or a genome. In embodiments, the target sequence or target gene includes coding sequence (DNA encoding a polypeptide, such as a structural protein or an enzyme), non-coding sequence, or both coding and non-coding sequence.

### A. Identification of Targets

There are numerous plant-endogenous targets (i.e., DNA sequence targets) for genome editing. The methods presented here can be applied to edit one or more genomic regions selected independently from the group consisting of a gene, an array of tandemly duplicated genes, a multigene family, an enhancer, a suppressor, a transcription factor binding site, a protein binding site, a promoter, a termination sequence, a splice acceptor sequence, a splice donor sequence, an intron, an exon, an siRNA, a sequence encoding a non-coding RNA, a microRNA, a transgene, an intergenic region, a genic region, a heterochromatic region, a euchromatic region, a region of methylated DNA, and a quantitative trait locus (QTL).

The method of the present invention may be used to introduce edits to affect any phenotype, quality, or trait of the organism. For instance, the methods herein may be used to introduce edits to the genome that affect yield, overall fitness, biomass, photosynthetic efficiency, nutrient use efficiency, heat tolerance, drought tolerance, herbicide tolerance, or disease resistance of a plant.

The methods presented here can be applied to a promoter bashing or fine-tuning approach, to create a range of phenotypes based on promoter alterations of a gene of a certain sequence or gene of interest (Rodriguez-Leal et al. Cell 2017, 171(2): 470-480). For example, a target gene may be selected that has a current, baseline level of expression in a target plant species. Guide RNAs may be produced that target different regions of the promoter of this target gene. Multiple lines of the elite germplasm may be generated containing distinct edits in the target gene promoter using the methods provided herein. For example, one line may have deleted a transcription factor binding site; a second line may have introduced a single base pair substitution in the transcription factor binding site; a third line may have introduced two base pair substitutions in the transcription factor binding site. The differentially edited promoters can be assessed for phenotype, including sub-organismal level phenotype such as RNA expression level, gene transcript splicing ratio, ribosomal occupancy, allele specific expression, metabolite abundance, protein modifications, micro RNA or small RNA abundance, protein abundance, or translational efficiency, and/or organismal level phenotype such as yield, overall fitness, biomass, photosynthetic efficiency, nutrient use efficiency, heat tolerance, drought tolerance, herbicide tolerance, disease resistance, salt tolerance, insect resistance, resistance against parasitic weeds, improved plant nutritional value, improved forage digestibility, increased grain yield, cytoplasmic male sterility, altered fruit ripening, increased storage life of plants or plant parts, reduced allergen production, and increased or decreased lignin content. In some embodiments, the edit results in increased transcription compared to the baseline level of expression in a target plant species. In some embodiments, the edit results in decreased transcription compared to the baseline level of expression in a target plant species. The optimal allele may be selected based on sub-organismal phenotype and/or organismal phenotype.

Any defective, deleterious, non-optimal, or underperforming allele found in elite germplasm can be edited to a non-deleterious or more optimal allele. In some embodiments, a target to be modified is a genetic variant that is known in the art to be deleterious. In some embodiments, a target to be modified is identified by a linkage study or an association study, such as a genome-wide association study (GWAS) or a transcriptome-wide association study (TWAS). In some embodiments, a target to be modified is identified through the use of statistical models, machine learning, or artificial intelligence. Deleterious genetic variants may be identified through analysis of factors including, but not limited to, evolutionary conservation (See e.g. Chun and Fay Genome Res 2009, 19:1553-1561; Rodgers-Melnick et al. PNAS 2015, 112:3823-3828), functional impact of amino acid change (See e.g. Ng et al. NAR 2003, 31:3812-3814; Adzhubei et al. Nat Methods 2010, 7:248-249), functional impact of protein conformation and/or stability (See e.g. Rosetta, a computational protein design platform from Cyrus Bio Inc.), adjacency to selective sweep regions (See e.g. Hufford et al. Nat Gen 2012, 44:808-813), and outlier status of a sub-organismal level phenotype such as RNA expression level, gene transcript splicing ratio, ribosomal occupancy, allele specific expression, metabolite abundance, protein modifications, micro RNA or small RNA abundance, protein abundance, or translational efficiency (See e.g. Zhao et al. AJHG 2016, 98:299-309).

Editing of coding sequences can be made using the methods disclosed herein to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. Proceedings of the World Congress on Vegetable Protein Utilization in Human Foods and Animal Feedstuffs, ed. Applewhite (American Oil Chemists Society, Champaign, Ill.) 1989, pp. 497-502; herein incorporated by reference); corn (Pedersen et al. J. Biol. Chem. 1986, 261:6279; Kirihara et al. Gene 1988, 71:359; both of which are herein incorporated by reference); and rice (Musumura et al. Plant Mol. Biol. 1989, 12:123, herein incorporated by reference). Other agronomically important genes encode latex, Floury 2, growth factors, seed storage factors, and transcription factors.

The methods disclosed herein can be used to modify herbicide resistance traits including genes coding for resistance to herbicides that act to inhibit the action of acetolactate synthase (ALS), in particular the sulfonylurea-type herbicides (e.g., the acetolactate synthase (ALS) gene containing DNA sequence modifications leading to such resistance, in particular the S4 and/or Hra modifications), genes coding for resistance to herbicides that act to inhibit action of glutamine synthase, such as phosphinothricin or basta (e.g., the bar gene); glyphosate (e.g., the EPSPS gene and the GAT gene; see, for example, U.S. Publication No. 20040082770 and WO 03/092360); or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron. Additional herbicide resistance traits are described for example in U.S. Patent Application 2016/0208243, herein incorporated by reference.

Sterility genes can also be modified and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development. Additional sterility traits are described for example in U.S. Patent Application 2016/0208243, herein incorporated by reference.

Genome editing can also be used to make haploid inducer lines as disclosed in WO2018086623 and US20190292553.

The quality of grain can be altered by modifying genes encoding traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be altered by modifying a gene or that could increase for example, starch for ethanol production, or provide expression of proteins. Another important commercial use of modified plants is the production of polymers and bioplastics such as described in U.S. Pat. No. 5,602,321. Genes such as beta-Ketothiolase, PHBase (polyhydroxyburyrate synthase), and acetoacetyl-CoA reductase (see Schubert et al. J. Bacteriol 1988, 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The methods disclosed herein can also be used for modification of native plant gene expression to achieve desirable plant traits, such as an agronomically desirable trait. Such traits include, for example, disease resistance, herbicide tolerance, drought tolerance, salt tolerance, insect resistance, resistance against parasitic weeds, improved plant nutritional value, improved forage digestibility, increased grain yield, cytoplasmic male sterility, altered fruit ripening, increased storage life of plants or plant parts, reduced allergen production, and increased or decreased lignin content. Genes capable of conferring these desirable traits are disclosed in U.S. Patent Application 2016/0208243, herein incorporated by reference.

In some embodiments, edits generated by the methods provided herein are evaluated for changes in phenotype on a sub-organismal level, including evaluation of RNA expression level, gene transcript splicing ratio, ribosomal occupancy, allele specific expression, metabolite abundance, protein modifications, micro RNA or small RNA abundance, protein abundance, and/or translational efficiency. In some embodiments, edits generated by the methods provided herein are evaluated for changes in phenotype on an organismal level, including yield, overall fitness, biomass, photosynthetic efficiency, nutrient use efficiency, heat tolerance, drought tolerance, herbicide tolerance, disease resistance, salt tolerance, insect resistance, resistance against parasitic weeds, improved plant nutritional value, improved forage digestibility, increased grain yield, cytoplasmic male sterility, altered fruit ripening, increased storage life of plants or plant parts, reduced allergen production, and increased or decreased lignin content. The optimal allele and/or edits may be selected based on sub-organismal phenotype and/or organismal phenotype.

The present disclosure may be used for genomic editing of any plant species, including, but not limited to, monocots and dicots (i.e., monocotyledons and dicotyledons, respectively). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), sesame (Sesamum spp.), flax (Linum usitatissimum), cannabis (Cannabis spp.), a vegetable crop, a forage crop, an industrial crop, a woody crop, a biomass crop, an ornamental, and a conifer.

In some embodiments, the graft is a heterograft. In other embodiments, the graft is a homograft. In some embodiments, the scion and the rootstock are different plant species. In some embodiments, the scion and the rootstock are the same plant species. In some embodiments, the scion and/or rootstock is a dicot. In some embodiments, the scion and/or rootstock is a monocot. In some embodiments, the scion is soy, canola, alfalfa, corn, oat, sorghum, sugarcane, banana, or wheat.

In some embodiments, the meristem is edited. In some embodiments, the genome of a meristem of a plant scion grafted onto a rootstock is edited.

### III. Delivery

### A. Vectors

Vectors are used to deliver nucleic acids to plant cells. In some embodiments, the vector is capable of autonomous replication within the host cell. In other embodiments, the vector is integrated into the genome of the host cell and replicated with the host genome. In some embodiments, termed “expression vectors”, the genes of the vector are expressed or are capable of being expressed under certain conditions. In some embodiments, the vector contains one or more regulatory elements operably linked to a gene. In some embodiments, the vector contains a promoter. In some embodiments, the promoter is a constitutive promoter, a conditional promoter, an inducible promoter, or a temporally or spatially specific promoter (e.g., a tissue specific promoter, a developmentally regulated promoter, or a cell cycle regulated promoter). In some embodiments, a vector is introduced to a host cell to produce RNA transcripts, proteins, or peptides within the host cell, as encoded by the contained nucleic acid.

In some embodiments of the method, the nucleic acid described herein can contained within any suitable plant transformation plasmid or vector. In some embodiments, the plant transformation plasmid or vector further comprises a selectable or screenable marker, such as but not limited to a fluorescent protein.

In embodiments of the method, the engineered system or a component thereof is delivered via at least one viral vector selected from the group consisting of adenoviruses, lentiviruses, adeno-associated viruses, retroviruses, geminiviruses, begomoviruses, tobamoviruses, potex viruses, comoviruses, wheat streak mosaic virus, barley stripe mosaic virus, bean yellow dwarf virus, bean pod mottle virus, cabbage leaf curl virus, beet curly top virus, tobacco yellow dwarf virus, tobacco rattle virus, potato virus X, and cowpea mosaic virus. In embodiments of the method, the engineered system or a component thereof is delivered via at least one bacterial vector capable of transforming a plant cell and selected from the group consisting of Agrobacterium sp., Rhizobium sp., Sinorhizobium (Ensifer) sp., Mesorhizobium sp., Bradyrhizobium sp., Azobacter sp., and Phyllobacterium sp. In some embodiments, a viral vector may be delivered to a plant by transformation with Agrobacterium.

In another embodiment, a T-DNA vector is used to deliver at least one nucleic acid to plant cells. In some embodiments, a T-DNA binary vector is used. In some embodiments, a T-DNA superbinary vector system is used. In other embodiments, a T-DNA ternary vector system is used. In some embodiments, the T-DNA system further comprises an additional virulence gene cluster. In some embodiments, the T-DNA system further comprises an accessory plasmid or virulence helper plasmid. In some embodiments, the T-DNA vector is an Agrobacterium vector.

In some embodiments, the T-DNA vector is an Agrobacterium rhizogenes vector. Agrobacterium rhizogenes, also known as Rhizobium rhizogenes, is a gram-negative soil bacteria that is capable of infecting the roots of a variety of plant species. Transformation of cells of the plant root with the Ri (root inducing) plasmid of the bacteria results in random integration of the genes from the Ri plasmid into the plant cell genome. This leads to expression of the genes from the Ri plasmid in the cells of the root, resulting in the host plant producing branching root overgrowth at the site of infection in what is known as “hairy root syndrome”. Replacement of the genes of the Ri plasmid with the desired transformation product, while maintaining the virulence genes, results in the ability to produce transgenic roots that are express the genes of the desired transformation product.

In some embodiments, the nucleic acid encoding the Cas nuclease and the nucleic acid encoding the guide RNA are provided in the same vector. In some embodiments, the nucleic acid encoding the Cas nuclease and the nucleic acid encoding the guide RNA are provided in different vectors. In some embodiments, the vector is a viral vector. In some embodiments, the vector is a viral vector or a T-DNA vector.

### B. Delivery of Genomic Modification System

The polynucleotides, ribonucleoproteins, DNA expression systems, engineered systems, and vectors (collectively referred to here as “genome editing reagents”) that are aspects of the invention can be delivered to a plant cell using various techniques and agents. In some embodiments, the plant cell is a cell of a rootstock. In some embodiments, the plant cell is a cell of a grafted scion. In some embodiments, the plant cell is a cell of a seed (including mature seed and immature seed). In some embodiments, the plant cell is a cell of a plant cutting. In some embodiments, the plant cell is a cell of a plant cell culture. In some embodiments, the plant cell is a cell of a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants). In some embodiments, the cell is a non-regenerable cell. In embodiments, one or more treatments is employed to deliver genome editing reagents into a plant cell or plant protoplast, e.g., through barriers such as a cell wall or a plasma membrane or nuclear envelope or other lipid bilayer. In an embodiment, genome editing reagents are delivered directly, for example by direct contact of the polynucleotide composition with a plant cell or plant protoplast. A genome editing reagent-containing composition in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant cell or plant protoplast (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing reagent-containing composition, whereby the genome editing reagent is delivered to the plant cell or plant protoplast. In embodiments, the genome editing reagent-containing composition is delivered using negative or positive pressure, for example, using vacuum infiltration or application of hydrodynamic or fluid pressure. In embodiments, the genome editing reagent-containing composition is introduced into a plant cell or plant protoplast e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in U.S. Published Patent Application 2014/0287509, incorporated by reference in its entirety herein. Other techniques useful for delivering the genome editing reagent-containing composition to a plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In embodiments, the genome editing reagent-containing composition is provided to a plant cell or plant protoplast by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the gRNA; see, e.g., Broothaerts et al. Nature 2005, 433:629-633. Any of these techniques or a combination thereof are alternatively employed on the plant part or tissue or intact plant (or seed) from which a plant cell or plant protoplast is optionally subsequently obtained or isolated; in embodiments, the genome editing reagent-containing composition is delivered in a separate step after the plant cell or plant protoplast has been obtained or isolated.

In embodiments, a treatment employed in delivery of a genome editing reagent to a plant cell or plant protoplast is carried out under a specific thermal regime, which can involve one or more appropriate temperatures, e.g., chilling or cold stress (exposure to temperatures below that at which normal growth of the plant cell or plant protoplast occurs), or heating or heat stress (exposure to temperatures above that at which normal growth of the plant cell or plant protoplast occurs), or treating at a combination of different temperatures. In embodiments, a specific thermal regime is carried out on a plant cell or plant protoplast, or on a plant or plant part from which a plant cell or plant protoplast is subsequently obtained or isolated, in one or more steps separate from the genome editing reagent delivery. In some embodiments, a specific thermal regime is carried out on a plant cell, wherein the plant cell is a cell of a rootstock. In some embodiments, a specific thermal regime is carried out on a plant cell, wherein the plant cell is a cell of a grafted scion. In some embodiments, a specific thermal regime is carried out on a plant cell, wherein the plant cell is a cell of a seed (including mature seed and immature seed). In some embodiments, a specific thermal regime is carried out on a plant cell, wherein the plant cell is a cell of a plant cutting. In some embodiments, a specific thermal regime is carried out on a plant cell, wherein the plant cell is a cell of a plant cell culture. In some embodiments, a specific thermal regime is carried out on a plant cell, wherein the plant cell is a cell of a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants).

In embodiments, a whole plant or plant part or seed, or an isolated plant cell or plant protoplast, or the plant or plant part from which a plant cell or plant protoplast is obtained or isolated, is treated with one or more delivery agents which can include at least one chemical, enzymatic, or physical agent, or a combination thereof. In embodiments, a genome editing reagent-containing composition further includes one or more one chemical, enzymatic, or physical agent for delivery. In some embodiments, the treated plant cell is a cell of a rootstock. In some embodiments, the treated plant cell is a cell of a grafted scion. In some embodiments, the treated plant cell is a cell of a seed (including mature seed and immature seed). In some embodiments, the treated plant cell is a cell of a plant cutting. In some embodiments, the treated plant cell is a cell of a plant cell culture. In some embodiments, the treated plant cell is a cell of a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants). Treatment with the chemical, enzymatic or physical agent can be carried out simultaneously with the genome editing reagent delivery, or in one or more separate steps that precede or follow the genome editing reagent delivery. In embodiments, a chemical, enzymatic, or physical agent, or a combination of these, is associated or complexed with a genome editing reagent composition; examples of such associations or complexes include those involving non-covalent interactions (e.g., ionic or electrostatic interactions, hydrophobic or hydrophilic interactions, formation of liposomes, micelles, or other heterogeneous composition) and covalent interactions (e.g., peptide bonds, bonds formed using cross-linking agents). In non-limiting examples, a genome editing reagent is provided as a liposomal complex with a cationic lipid, or as a complex with a carbon nanotube, or as a fusion protein between the nuclease and a cell-penetrating peptide. Examples of agents useful for delivering a genome editing reagent include the various cationic liposomes and polymer nanoparticles reviewed by Zhang et al. (2007) J Controlled Release, 123:1-10, and the cross-linked multilamellar liposomes described in U.S. Patent Application Publication 2014/0356414 A1, incorporated by reference in its entirety herein.

Compositions comprising: (i) RNA molecules comprising an MTS operably linked to a Cas nuclease and/or guide RNA(s); (ii) nucleic acids encoding RNA guided nucleases; and/or (iii) donor DNA templates can further comprise components that include:
(a) solvents (e.g., water, dimethylsulfoxide, dimethylformamide, acetonitrile, N-pyrrolidine, pyridine, hexamethylphosphoramide, alcohols, alkanes, alkenes, dioxanes, polyethylene glycol, and other solvents miscible or emulsifiable with water or that will dissolve phosphonucleotides in non-aqueous systems);
(b) fluorocarbons (e.g., perfluorodecalin, perfluoromethyldecalin);
(c) glycols or polyols (e.g., propylene glycol, polyethylene glycol);
(d) surfactants, including cationic surfactants, anionic surfactants, non-ionic surfactants, and amphiphilic surfactants, e.g., alkyl or aryl sulfates, phosphates, sulfonates, or carboxylates; primary, secondary, or tertiary amines; quaternary ammonium salts; sultaines, betaines; cationic lipids; phospholipids; tallowamine; bile acids such as cholic acid; saponins or glycosylated triterpenoids or glycosylated sterols (e.g., saponin commercially available as catalogue number 47036-50g-F, Sigma-Aldrich, St. Louis, MO); long chain alcohols; organosilicone surfactants including nonionic organosilicone surfactants such as trisiloxane ethoxylate surfactants or a silicone polyether copolymer such as a copolymer of polyalkylene oxide modified heptamethyl trisiloxane and allyloxypolypropylene glycol methylether (commercially available as SILWET L-77TM brand surfactant having CAS Number 27306-78-1 and EPA Number CAL. REG. NO. 5905-50073-AA, Momentive Performance Materials, Inc., Albany, N.Y.); specific examples of useful surfactants include sodium lauryl sulfate, the Tween series of surfactants, Triton-X100, Triton-X114, CHAPS and CHAPSO, Tergitol-type NP-40, and Nonidet P-40;
(e) lipids, lipoproteins, lipopolysaccharides;
(f) acids, bases, caustic agents; buffers;
(g) peptides, proteins, or enzymes (e.g., cellulase, pectolyase, maceroenzyme, pectinase), including cell-penetrating or pore-forming peptides (e.g., (BO100) 2K8, Genscript; poly-lysine, poly-arginine, or poly-homoarginine peptides; gamma zein, see US Patent Application publication 2011/0247100, incorporated herein by reference in its entirety; transcription activator of human immunodeficiency virus type 1 (“HIV-1 Tat”) and other Tat proteins, see, e.g., www[dot]lifetein[dot]com/Cell_Penetrating_Peptides[dot]html and Järver Mol. Therapy-Nucleic Acids 2012, 1: e27,1-17); octa-arginine or nona-arginine; poly-homoarginine (see Unnamalai et al. FEBS Letters 2004, 566:307-310); see also the database of cell-penetrating peptides CPPsite 2.0 publicly available at webs[dot]iiitd[dot]edu[dot]in/Raghava/cppsite (Kardani and Bolhassani J Mol Biol 2021, 433(11): 166703)
(h) RNase inhibitors;
(i) cationic branched or linear polymers such as chitosan, poly-lysine, DEAE-dextran, polyvinylpyrrolidone (“PVP”), or polyethylenimine (“PEI”, e.g., PEI, branched, MW 25,000, CAS #9002-98-6; PEI, linear, MW 5000, CAS #9002-98-6; PEI linear, MW 2500, CAS #9002-98-6);
(j) dendrimers (see, e.g., US Patent Application Publication 2011/0093982, incorporated herein by reference in its entirety);
(k) counter-ions, amines or polyamines (e.g., spermine, spermidine, putrescine), osmolytes, buffers, and salts (e.g., calcium phosphate, ammonium phosphate);
(l) polynucleotides (e.g., non-specific double-stranded DNA, salmon sperm DNA);
(m) transfection agents (e.g., Lipofectin®, Lipofectamine®, and Oligofectamine®, and Invivofectamine® (all from Thermo Fisher Scientific, Waltham, MA), PepFect (see Ezzat et al. Nucleic Acids Res. 2011, 39:5284-5298), TransIt® transfection reagents (Mirus Bio, LLC, Madison, WI), and poly-lysine, poly-homoarginine, and poly-arginine molecules including octo-arginine and nono-arginine as described in Lu et al. J. Agric. Food Chem. 2010, 58:2288-2294);
(n) antibiotics, including non-specific DNA double-strand-break-inducing agents (e.g., phleomycin, bleomycin, talisomycin);
(o) antioxidants (e.g., glutathione, dithiothreitol, ascorbate); and/or
(p) chelating agents (e.g., EDTA, EGTA).

In embodiments, the chemical agent is provided simultaneously with the genome editing reagent. In embodiments, the genome editing reagent is covalently or non-covalently linked or complexed with one or more chemical agent; for example, a polynucleotide genome editing reagent can be covalently linked to a peptide or protein (e.g., a cell-penetrating peptide or a pore-forming peptide) or non-covalently complexed with cationic lipids, polycations (e.g., polyamines), or cationic polymers (e.g., PEI). In embodiments, the genome editing reagent is complexed with one or more chemical agents to form, e.g., a solution, liposome, micelle, emulsion, reverse emulsion, suspension, colloid, or gel.

In embodiments, the physical agent is at least one selected from the group consisting of particles or nanoparticles (e.g., particles or nanoparticles made of materials such as carbon, silicon, silicon carbide, gold, tungsten, polymers, or ceramics) in various size ranges and shapes, magnetic particles or nanoparticles (e.g., silenceMag Magnetotransfection™ agent, OZ Biosciences, San Diego, Calif.), abrasive or scarifying agents, needles or microneedles, matrices, and grids. In embodiments, particulates and nanoparticulates are useful in delivery of the polynucleotide composition or the nuclease or both. Useful particulates and nanoparticles include those made of metals (e.g., gold, silver, tungsten, iron, cerium), ceramics (e.g., aluminum oxide, silicon carbide, silicon nitride, tungsten carbide), polymers (e.g., polystyrene, polydiacetylene, and poly(3,4-ethylenedioxythiophene) hydrate), semiconductors (e.g., quantum dots), silicon (e.g., silicon carbide), carbon (e.g., graphite, graphene, graphene oxide, or carbon nanosheets, nanocomplexes, or nanotubes), and composites (e.g., polyvinylcarbazole/graphene, polystyrene/graphene, platinum/graphene, palladium/graphene nanocomposites). In embodiments, such particulates and nanoparticulates are further covalently or non-covalently functionalized, or further include modifiers or cross-linked materials such as polymers (e.g., linear or branched polyethylenimine, poly-lysine), polynucleotides (e.g., DNA or RNA), polysaccharides, lipids, polyglycols (e.g., polyethylene glycol, thiolated polyethylene glycol), polypeptides or proteins, and detectable labels (e.g., a fluorophore, an antigen, an antibody, or a quantum dot). In various embodiments, such particulates and nanoparticles are neutral, or carry a positive charge, or carry a negative charge. Embodiments of compositions including particulates include those formulated, e.g., as liquids, colloids, dispersions, suspensions, aerosols, gels, and solids. Embodiments include nanoparticles affixed to a surface or support, e.g., an array of carbon nanotubes vertically aligned on a silicon or copper wafer substrate. Embodiments include polynucleotide compositions including particulates (e.g., gold or tungsten or magnetic particles) delivered by a Biolistic-type technique or with magnetic force. The size of the particles used in Biolistics is generally in the “microparticle” range, for example, gold microcarriers in the 0.6, 1.0, and 1.6 micrometer size ranges (see, e.g., instruction manual for the Helios® Gene Gun System, Bio-Rad, Hercules, Calif.; Randolph-Anderson et al. (2015) “Sub-micron gold particles are superior to larger particles for efficient Biolistic® transformation of organelles and some cell types”, Bio-Rad US/EG Bulletin 2015), but successful Biolistics delivery using larger (40 nanometer) nanoparticles has been reported in cultured animal cells; see O'Brian and Lummis (2011) BMC Biotechnol., 11:66-71. Other embodiments of useful particulates are nanoparticles, which are generally in the nanometer (nm) size range or less than 1 micrometer, e.g., with a diameter of less than about 1 nm, less than about 3 nm, less than about 5 nm, less than about 10 nm, less than about 20 nm, less than about 40 nm, less than about 60 nm, less than about 80 nm, and less than about 100 nm. Specific, non-limiting embodiments of nanoparticles commercially available (all from Sigma-Aldrich Corp., St. Louis, Mo.) include gold nanoparticles with diameters of 5, 10, or 15 nm; silver nanoparticles with particle sizes of 10, 20, 40, 60, or 100 nm; palladium “nanopowder” of less than 25 nm particle size; single-, double-, and multi-walled carbon nanotubes, e.g., with diameters of 0.7-1.1, 1.3-2.3, 0.7-0.9, or 0.7-1.3 nm, or with nanotube bundle dimensions of 2-10 nm by 1-5 micrometers, 6-9 nm by 5 micrometers, 7-15 nm by 0.5-10 micrometers, 7-12 nm by 0.5-10 micrometers, 110-170 nm by 5-9 micrometers, 6-13 nm by 2.5-20 micrometers. Embodiments include genome editing reagent-containing compositions including materials such as gold, silicon, cerium, or carbon, e.g., gold or gold-coated nanoparticles, silicon carbide whiskers, carborundum, porous silica nanoparticles, gelatin/silica nanoparticles, nanoceria or cerium oxide nanoparticles (CNPs), carbon nanotubes (CNTs) such as single-, double-, or multi-walled carbon nanotubes and their chemically functionalized versions (e.g., carbon nanotubes functionalized with amide, amino, carboxylic acid, sulfonic acid, or polyethylene glycol moieties), and graphene or graphene oxide or graphene complexes; see, for example, Wong et al. (2016) Nano Lett., 16:1161-1172; Giraldo et al. (2014) Nature Materials, 13:400-409; Shen et al. (2012) Theranostics, 2:283-294; Kim et al. (2011) Bioconjugate Chem., 22:2558-2567; Wang et al. (2010) J. Am. Chem. Soc. Comm., 132:9274-9276; Zhao et al. (2016) Nanoscale Res. Lett., 11:195-203; and Choi et al. (2016) J. Controlled Release, 235:222-235. See also, for example, the various types of particles and nanoparticles, their preparation, and methods for their use, e.g., in delivering polynucleotides and polypeptides to cells, disclosed in U.S. Patent Application Publications 2010/0311168, 2012/0023619, 2012/0244569, 2013/0145488, 2013/0185823, 2014/0096284, 2015/0040268, 2015/0047074, and 2015/0208663, all of which are incorporated herein by reference in their entirety.

In embodiments, a genome editing reagent is delivered to plant cells or plant protoplasts prepared or obtained from a plant, plant part, or plant tissue that has been treated with the polynucleotide compositions (and optionally the nuclease). In some embodiments, the treated plant cell is a cell of a rootstock. In some embodiments, the treated plant cell is a cell of a grafted scion. In some embodiments, the treated plant cell is a cell of a seed (including mature seed and immature seed). In some embodiments, the treated plant cell is a cell of a plant cutting. In some embodiments, the treated plant cell is a cell of a plant cell culture. In some embodiments, the treated plant cell is a cell of a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants). In embodiments, one or more one chemical, enzymatic, or physical agent, separately or in combination with the genome editing reagent, is provided/applied at a location in the plant or plant part other than the plant location, part, or tissue from which the plant cell or plant protoplast is obtained or isolated. In embodiments, the genome editing reagent is applied to adjacent or distal cells or tissues and is transported (e.g., through the vascular system or by cell-to-cell movement) to the meristem from which plant cells or plant protoplasts are subsequently isolated. In embodiments, a genome editing reagent-containing composition is applied by soaking a seed or seed fragment or zygotic or somatic embryo in the genome editing reagent-containing composition, whereby the genome editing reagent is delivered to the seed or seed fragment or zygotic or somatic embryo from which plant cells or plant protoplasts are subsequently isolated. In embodiments, a flower bud or shoot tip is contacted with a genome editing reagent-containing composition, whereby the genome editing reagent is delivered to cells in the flower bud or shoot tip from which plant cells or plant protoplasts are subsequently isolated. In embodiments, a genome editing reagent-containing composition is applied to the surface of a plant or of a part of a plant (e.g., a leaf surface), whereby the genome editing reagent is delivered to tissues of the plant from which plant cells or plant protoplasts are subsequently isolated. In embodiments a whole plant or plant tissue is subjected to particle- or nanoparticle-mediated delivery (e.g., Biolistics or carbon nanotube or nanoparticle delivery) of a genome editing reagent-containing composition, whereby the genome editing reagent is delivered to cells or tissues from which plant cells or plant protoplasts are subsequently isolated.

Compositions comprising: (i) RNA molecules comprising an MTS operably linked to a Cas nuclease and/or guide RNA(s); (ii) nucleic acids encoding RNA guided nucleases; and/or (iii) donor DNA templates can be delivered to the plant and/or meristem cells of the plant by particle mediated delivery, and any other direct method of delivery, such as but not limiting to, Agrobacterium-mediated transformation, polyethylene glycol (PEG)-mediated transfection to protoplasts, whiskers mediated transformation, electroporation, particle bombardment, and/or by use of cell-penetrating peptides. In some embodiments, the plant cell to which the composition is delivered is a cell of a rootstock. In some embodiments, the plant cell to which the composition is delivered is a cell of a grafted scion. In some embodiments, the plant cell to which the composition is delivered is a cell of a seed (including mature seed and immature seed). In some embodiments, the plant cell to which the composition is delivered is a cell of a plant cutting. In some embodiments, the plant cell to which the composition is delivered is a cell of a plant cell culture. In some embodiments, the plant cell to which the composition is delivered is a cell of a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants).

In certain embodiments, plants are contacted either simultaneously or sequentially with one, two, three or more RNA molecules in one or more compositions where at least one of the RNA molecules comprises a guide RNA fused to an MTS. In some embodiments, the composition contacts a rootstock. In some embodiments, the composition contacts a grafted scion. In some embodiments, the composition contacts a seed (including mature seed and immature seed). In some embodiments, the composition contacts a plant cutting. In some embodiments, the composition contacts a plant cell culture. In some embodiments, the composition contacts a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants). In certain embodiments, plants are contacted either simultaneously or sequentially with one, two, three or more RNA molecules in one or more compositions where at least one of the RNA molecules comprises an RNA encoding a Cas nuclease fused to an MTS. In certain embodiments, one of the RNA molecules comprises a guide RNA fused to an MTS and a second RNA molecule comprises RNA encoding an RNA guided Cas nuclease and optionally an MTS, where the RNA guided Cas nuclease can process the RNA comprising the guide RNA to release a functional guide RNA. In certain embodiments, one of the RNA molecules comprises at least one guide RNA fused to an MTS and a second RNA molecule comprises RNA encoding an RNA guided nuclease and optionally an MTS, where the RNA guided nuclease cannot process the RNA comprising the guide RNA to release a functional guide RNA (e.g., processing elements present in the RNA molecule comprising the gRNA and the MTS are not recognized by the RNA-guided nuclease). In certain embodiments, guide RNAs of the first and second RNA molecule are flanked by or comprise processing elements (e.g., DRs) which are processed by different RNA-guided nuclease (e.g., a Cas12a nuclease can process the first RNA molecule and a Cas12j nuclease can process the second RNA molecule). In certain embodiments, the guide RNA(s) of the first RNA molecule distinct from the guide RNA(s) of the second RNA molecule. Such distinct gRNAs provided by the first RNA molecule can provide for genome editing at one or more first genomic sites in a meristem cell while the distinct gRNAs provided by the second RNA molecule can provide for genome editing at one or more second genomic sites in a meristem cell. Such contacting the plant with RNA molecules in a composition can occur sequentially such that the first gRNA(s) are delivered, allowed sufficient time (e.g., about 6, 12, 18, or 20 to about 24, 30, or 36 hours) to effect desired genome edits, followed by contacting the plant with the second RNA molecules in a second composition to deliver the second gRNA(s) to effect additional desired genome edits, where such desired genome edits are effected by providing the gRNA(s) and an RNA guided nuclease in at least the meristem cell. Without seeking to be limited by theory, it is believed that cutting chromosomes at multiple location simultaneously is cytotoxic and that such cytotoxicity can be mitigated by delivering a limited number of guide RNAs at different times (e.g., about 6, 12, 18, or 20 to about 24, 30, or 36 hours apart). In certain embodiments, a plant can be contacted by one or more RNA molecules that comprise at least one gRNA fused to an MTS, optionally along with an RNA encoding RNA guided Cas nuclease, permitted a sufficient period of time to accumulate the RNA molecule in the meristem cells (e.g., about 6, 12, 18 or 20 to about 24, 30, or 36 hours apart), and then contacted with a different mixture of one or more RNA molecules that comprise at least one different gRNA fused to an MTS, optionally along with an RNA encoding an RNA guided Cas nuclease, where the RNA guided Cas nuclease can process the RNA comprising the guide RNA to release a functional guide RNA and/or effect a desired genomic edit with the gRNA in the meristem cells.

Guide RNAs can be provided to at least the meristem cell by a variety of methods that include stable expression with an integrated transgene, expression from a viral vector, or transient expression such as by introducing an RNA that encodes the gRNA or a DNA that encodes the gRNA that is operably linked to an MTS. In certain embodiments, the gRNA is predominantly localized in meristem tissue of the plant. Delivery of RNAs encoding the gRNA(s) or DNA(s) that encode those gRNA(s) to the plant and/or meristem cells of the plant can be achieved by particle mediated delivery, and any other direct method of delivery, such as but not limited to, Agrobacterium-mediated transformation, polyethylene glycol (PEG)-mediated transfection to protoplasts, whiskers mediated transformation, electroporation, particle bombardment, and/or by use of cell-penetrating peptides. In some embodiments, the gRNA(s) are delivered to a rootstock. In some embodiments, the gRNA(s) are delivered to a grafted scion. In some embodiments, the gRNA(s) are delivered to a seed (including mature seed and immature seed). In some embodiments, the gRNA(s) are delivered to a plant cutting. In some embodiments, the gRNA(s) are delivered to a plant cell culture. In some embodiments, the gRNA(s) are delivered to a plant organ (e.g., intact nodal bud, shoot apex or shoot apical meristem, root apex or root apical meristem, lateral meristem, intercalary meristem, zygotic embryo, somatic embryo, ovule, pollen, microspore, anther, hypocotyl, cotyledon, leaf, petiole, stem, tuber, root, flowers, fruits, shoots, and explants).

In some embodiments, a guide RNA for the Cas nuclease is applied to a leaf, a shoot, a stem, and/or meristem of the plant. In some embodiments, a composition comprising the guide RNA for the Cas nuclease is applied to a leaf, a shoot, a stem, and/or meristem of the plant. In some embodiments, the composition comprising the guide RNA comprises a nuclease inhibitor. In some embodiments, the composition comprising the guide RNA comprises an RNase inhibitor.

In some embodiments, delivery of the guide RNA comprises spraying a the guide RNA.

In some embodiments, delivery of the guide RNA comprises rubbing a composition comprising the guide RNA onto the leaves, shoot, stem, and/or meristem.

In some embodiments, delivery of the guide RNA comprises injecting a composition comprising the guide RNA into the stem.

In some embodiments, delivery of the guide RNA comprises leaf infiltration of a composition comprising the guide RNA into a leaf. In some embodiments, the leaf infiltration comprises forced infiltration using a needle-less syringe or vacuum pump.

In some embodiments, delivery of a guide RNA for the Cas nuclease comprises biolistic transformation of nucleic acid encoding the guide RNA into the leaf, shoot, stem, and/or meristem. In some embodiments, the biolistic transformation comprises transformation of circular DNA encoding the guide RNA.

In other embodiments, a guide RNA for the Cas nuclease is delivered to the roots of the plant. In some embodiments, a composition comprising the guide RNA for the Cas nuclease is applied to the roots. In some embodiments, the composition comprising the guide RNA comprises a nuclease inhibitor. In some embodiments, the composition comprising the guide RNA comprises an RNase inhibitor.

In some embodiments, the guide RNA is delivered to the plant root by incubating the root with a composition comprising the guide RNA.

In some embodiments, a guide RNA for the Cas nuclease is delivered to the plant root by Agrobacterium rhizogenes transformation.

RNA guided nucleases can be provided to at least the meristem cell by a variety of methods that include stable expression with an integrated transgene, expression from a viral vector, or transient expression such as by introducing an RNA that encodes the RNA guided nuclease or an RNA that encodes the RNA guided nuclease that is operably linked to an MTS. In certain embodiments, an active form of the RNA guided nuclease is predominantly localized in root tissue of the plant. In certain embodiments, the RNA guided nuclease can be operably linked to a vegetative stage, root-preferred or root-specific promoter including but not limited to those disclosed in U.S. Pat. Nos. 8,058,419; 10,533,184; Khandal et al. Plant Biotechnol J 2020, 18:2225-2240; Xu et al. Plant Biotechnol J 2020, 18:1585-1597; and James et al. Front Plant Sci 2022, 13:1009487.

In some embodiments, a plant expressing transgenically a Cas polypeptide may be genomically edited by delivery of a second RNA containing only guide RNAs suitable for the transgenically expressed Cas polypeptide.

The RNA sequences are generally made and assembled at first in DNA form as RNA expressing vectors using recombinant DNA technology. RNA expression is performed in vitro, and the RNA purified according to well established methods. Addition of 5′ caps and polyA tails to mRNAs can be performed according to methods established in the literature. Alternatively, some RNAs designed as described can be purchased from commercial providers.

A substantially purified RNA composition is understood to comprise a high concentration of an RNA molecule of interest, although in some cases it may comprise two distinct RNAs. For example, one RNA may comprise a Cas nuclease while another may comprise a corresponding guide or guide array. In addition, a substantially purified RNA composition may comprise other added components, such as a pH buffer, salt, surfactants, and/or RNase inhibitors.

Plants can be effectively contacted with the RNA vectors in many ways. Often it will be convenient to load them into the phloem of plants through the leaves, for example by nicking a leaf and submerging the injured tissue into a solution of substantially purified RNAs. Other avenues are also possible, such as by injection into the stems with a needle or use of a handheld biolistics device. In some embodiments, a surfactant is added to the purified RNA, and the liquid is applied to a tissue like embryonic shoot, leaf, stem, or inflorescence, with or without slight injury such as scratching.

The RNAs are often applied at the vegetative stage of the life cycle of a plant, so as to reach vegetative meristems before they convert to floral meristems. In some cases, however, it may be convenient to apply the vectors, RNA molecules, or compositions comprising the RNA molecules or vectors, to floral meristems, especially at early stages of differentiation. In certain embodiments, a soybean plant is contacted at the vegetative stage with a composition comprising the RNA molecules or vectors at vegetative stage Ve, V1, or V2 to about the V4 V (n) stage where 1, 2, 3, 4, or n is the number of trifoliate leaves (Soybean Growth and Development, M. Licht, 2014, Iowa State University Extension and Outreach, P M 1945). In certain embodiments, a maize plant is contacted at the vegetative stage with a composition comprising the RNA molecules or vectors at vegetative stage Ve, V1, or V2 to about the V4 V(n) stage (Corn Growth Stages, M. Licht, Iowa State University Extension and Outreach, on the https internet site “crops[dot]extension[dot]iastate[dot]edu/encyclopedia/corn-growth-stages”).

### EMBODIMENTS

1. A method of editing a genomic target in a scion comprising
grafting the scion onto a rootstock comprising nucleic acid encoding a Cas9 nickase or Cas12 nuclease and nucleic acid encoding a guide RNA for the Cas9 nickase or Cas12 nuclease, wherein the nucleic acid encoding the guide RNA and the nucleic acid encoding the Cas9 nickase or the Cas12 nuclease are fused to nucleic acid encoding a meristem transport segment (MTS).

2. The method of embodiment 1 wherein the Cas9 nickase or Cas12 nuclease is associated with a reverse transcriptase.

3. The method of embodiment 2, wherein the Cas9 nickase or Cas12 nuclease is fused to the reverse transcriptase.

4. The method of any one of embodiments 1-3, wherein the guide RNA comprises at its 3′ end a priming site and an edit to be incorporated into the genomic target.

5. The method of any one of embodiments 1-4, wherein the Cas12 nuclease is a Cas12 nickase.

6. The method of embodiment 5, wherein the Cas12 nickase comprises mutation in one or more nuclease active sites compared to a wildtype Cas12.

7. The method of any one of embodiments 1-6, wherein RNA encoding the Cas9 nickase or Cas12 nuclease and the guide RNA are transported from the rootstock to the scion by the plant vascular system.

8. The method of any one of embodiments 1-7, wherein RNA encoding the Cas9 nickase or Cas12 nuclease and the guide RNA are transported to the scion through the phloem.

9. The method of any one of embodiments 1-8, wherein RNA encoding the Cas9 nickase or Cas12 nuclease is translated in the scion.

10. The method of any one of embodiments 1-9, wherein the genome of a cell in a meristem of the scion is edited.

11. The method of any one of embodiments 1-10, wherein the nucleic acid encoding the Cas9 nickase or Cas12 nuclease and the nucleic acid encoding the guide RNA are provided to the rootstock in the same vector.

12. The method of any one of embodiments 1-10, wherein the nucleic acid encoding the Cas9 nickase or Cas12 nuclease and the nucleic acid encoding the guide RNA are provided to the rootstock in different vectors.

13. The method of embodiment 11 or embodiment 12, wherein the vector is a viral vector or a T-DNA vector.

14. The method of any one of embodiments 1-13, wherein the scion and the rootstock are different plant species.

15. The method of any one of embodiments 1-13, wherein the scion and the rootstock are the same plant species.

16. The method of any one of embodiments 1-15, wherein the scion and/or rootstock is a dicot.

17. The method of any one of embodiments 1-15, wherein the scion and/or rootstock is a monocot.

18. The method of any one of embodiments 1-17, wherein the scion is soy, canola, alfalfa, corn, oat, sorghum, sugarcane, banana or wheat.

19. The method of any one of embodiments 1-18, wherein the MTS comprises:
i. a Flowering Locus T (FT)-derived sequence, a tRNA like sequence (TLS), a meristem transport component (MTC); or
ii. an RNA hairpin comprising a first stem of 8 to 12 nucleotides, at least one variable bulge, a second stem of 4 to 7 nucleotides, and a variable loop.

20. The method of embodiment 19, wherein the MTS comprises an FT-derived sequence, and wherein the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24.

21. The method of embodiment 19, wherein the MTS comprises a TLS, and wherein the TLS comprises the nucleotide sequence set forth in SEQ ID NO: 29 or 30.

22. The method of any one of embodiments 1-21, wherein the nucleic acid encoding the MTS is located 3′ of the nucleic acid encoding the Cas9 nickase or Cas12 nuclease and/or 3′ of the nucleic acid encoding the guide RNA.

23. The method of any one of embodiments 1-21, wherein the nucleic acid encoding the MTS is located 5′ of the nucleic acid encoding the Cas9 nickase or Cas12 nuclease and/or 5′ of the nucleic acid encoding the guide RNA.

24. The method of any one of embodiments 1-23, wherein the nucleic acid encoding the Cas9 nickase or Cas12 nuclease is operably linked to a promoter.

25. The method of embodiment 24, wherein the promoter is active in roots and/or phloem companion cells.

26. The method of embodiment 24, wherein the promoter is the promoter of a gene selected from Arabidopsis WRKY6, chickpea WRKY31, carrot MYB113, corn GLU1, strawberry RB7-type TIP-2, and banana TIP2-2, or the promoter of an orthologous gene thereof.

27. The method of embodiment 24, wherein the promoter is selected from a promoter from a Flowering Locus T (FT) gene, a promoter from a Fabaceaen FOR1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter.

28. The method of any one of embodiments 1-27, wherein the nucleic acid encoding the Cas9 nickase or Cas12 nuclease is codon-optimized for expression in dicots.

29. The method of any one of embodiments 1-27, wherein the nucleic acid encoding the Cas9 nickase or Cas12 nuclease is codon-optimized for expression in monocots.

30. The method of any one of embodiments 1-27, wherein the nucleic acid encoding the Cas9 nickase or Cas12 nuclease is codon-optimized for expression in corn, soy or wheat.

31. The method of any one of embodiments 1-30, wherein the nucleic acid encoding the guide RNA is operably linked to a promoter.

32. The method of embodiment 31, wherein the promoter is an RNA polymerase II promoter or an RNA polymerase III promoter.

33. The method of embodiment 32, wherein the RNA polymerase II promoter or RNA polymerase III promoter is endogenous to the species of the rootstock.

34. The method of any one of embodiments 1-33, wherein the nucleic acid encoding the guide RNA and the MTS is located between two ribozyme sequences.

35. The method of embodiment 34, wherein each of the ribozyme sequences is independently selected from a hammerhead ribozyme sequence, a HDV ribozyme sequence, a Csy4 sequence, and a tRNA processing enzyme sequence.

36. The method of any one of embodiments 1-35, wherein the nucleic acid encoding the guide RNA and the MTS further comprises a hammerhead ribozyme sequence 5′ to the nucleic acid encoding the guide RNA and the MTS, and an HDV ribozyme 3′ to the nucleic acid encoding the guide RNA and the MTS.

37. The method of any one of embodiments 1-36, wherein the nucleic acid encoding the guide RNA and the MTS further comprises a terminator.

38. The method of embodiment 37, wherein the terminator is a U6 terminator.

39. The method of any one of embodiments 1-38, wherein the rootstock comprises nucleic acid encoding two, three, four, five, or more than five guide RNAs.

40. The method of embodiment 39, wherein the nucleic acid encoding each of the two, three, four, five, or more than five guide RNAs is joined to a nucleic acid encoding an MTS.

41. The method of any one of embodiments 1-40, wherein the Cas12 nuclease is selected from Cas12a (Cpf1), Cas12e (CasX), Cas12d (CasY), Cas12h, Cas12i, and Cas12j.

42. The method of any one of embodiments 1-41, wherein the rootstock further comprises nucleic acid encoding a detectable marker fused to a nucleic acid encoding an MTS.

43. The method of any one of embodiments 1-42, further comprising retrieving a progeny of the scion, wherein the progeny has an altered genome.

44. The method of any one of embodiments 1-43, wherein two or more guide RNAs are encoded by a single precursor RNA.

45. The method of embodiment 44, wherein the two or more guide RNAs are each flanked by a direct repeat.

46. An edited plant produced by the method of any one of embodiments 1-45.

47. A rootstock comprising:
nucleic acid encoding a Cas9 nickase or Cas12 nuclease, and
nucleic acid encoding a guide RNA for the Cas9 nickase or Cas12 nuclease,
wherein the nucleic acid encoding the guide RNA and the nucleic acid encoding the Cas9 nickase or Cas12 nuclease are fused to nucleic acid encoding a meristem transport segment (MTS).

48. The rootstock of embodiment 47, wherein the MTS comprises:
(i) a Flowering Locus T (FT)-derived sequence, a tRNA like sequence (TLS), a meristem transport component (MTC); or
(ii) an RNA hairpin comprising a first stem of 8 to 12 nucleotides, at least one variable bulge, a second stem of 4 to 7 nucleotides, and a variable loop.

49. The rootstock of embodiment 48, wherein the MTS comprises an FT-derived sequence, and wherein the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24.

50. The rootstock of embodiment 48, wherein the MTS comprises a TLS, and wherein the TLS comprises the nucleotide sequence set forth in SEQ ID NO: 29 or 30.

51. The rootstock of any one of embodiments 47-50, wherein the nucleic acid encoding the MTS is located 3′ of the nucleic acid encoding the Cas9 nickase or Cas12 nuclease and/or 3′ of nucleic acid encoding the guide RNA.

52. The rootstock of any one of embodiments 47-50, wherein the nucleic acid encoding the MTS is located 5′ of the nucleic acid encoding the Cas9 nickase or Cas12 nuclease and/or 5′ of the nucleic acid encoding the guide RNA.

53. The rootstock of any one of embodiments 47-52, wherein the nucleic acid encoding the Cas9 nickase or Cas12 nuclease is operably linked to a promoter.

54. The rootstock of embodiment 53, wherein the promoter is active in roots and/or phloem companion cells.

55. The rootstock of embodiment 53, wherein the promoter is the promoter of a gene selected from Arabidopsis WRKY6, chickpea WRKY31, carrot MYB113, corn GLU1, strawberry RB7-type TIP-2, and banana TIP2-2, or the promoter of an orthologous gene thereof.

56. The rootstock of embodiment 53, wherein the promoter is selected from a promoter from a Flowering Locus T (FT) gene, a promoter from a Fabaceaen FOR1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter.

57. The rootstock of any one of embodiments 47-56, wherein the nucleic acid encoding the Cas9 nickase or Cas12 nuclease is codon-optimized for expression in dicots.

58. The rootstock of any one of embodiments 47-56, wherein the nucleic acid encoding the Cas9 nickase or Cas12 nuclease is codon-optimized for expression in monocots.

59. The rootstock of any one of embodiments 47-58, wherein the nucleic acid encoding the Cas9 nickase or Cas12 nuclease is codon-optimized for expression in corn, soy or wheat.

60. The rootstock of any one of embodiments 47-59, wherein the nucleic acid encoding the guide RNA is operably linked to a promoter.

61. The rootstock of embodiment 60, wherein the promoter is an RNA polymerase II promoter or an RNA polymerase III promoter.

62. The rootstock of embodiment 61, wherein the RNA polymerase II promoter or RNA polymerase III promoter is endogenous to the species of the rootstock.

63. The rootstock of any one of embodiments 47-62, wherein the nucleic acid encoding the guide RNA and the MTS is located between two ribozyme sequences.

64. The rootstock of embodiment 63, wherein each of the ribozyme sequences is independently selected from a hammerhead ribozyme sequence, a HDV ribozyme sequence, a Csy4 sequence, and a tRNA processing enzyme sequence.

65. The rootstock of any one of embodiments 47-62, wherein the nucleic acid encoding the guide RNA and the MTS further comprises a hammerhead ribozyme sequence 5′ to the nucleic acid encoding the guide RNA and the MTS, and a HDV ribozyme 3′ to the nucleic acid encoding the guide RNA and the MTS.

66. The rootstock of any one of embodiments 47-65, wherein the nucleic acid encoding the guide RNA and the MTS further comprises a terminator.

67. The rootstock of embodiment 66, wherein the terminator is a U6 terminator.

68. The rootstock of any one of embodiments 47-67, wherein the rootstock comprises nucleic acid encoding two, three, four, five, or more than five guide RNAs.

69. The rootstock of embodiment 68, wherein the nucleic acid encoding each of the two, three, four, five, or more than five guide RNAs is joined to an MTS.

70. The rootstock of any one of embodiments 47-69, wherein the Cas12 nuclease is selected from Cas12a (Cpf1), Cas12e (CasX), Cas12d (CasY), Cas12h, Cas12i, and Cas12j.

71. An edited plant genome of the plant of embodiment 46.

72. A non-regenerable plant cell, tissue, or plant part of the plant of embodiment 46.

### EXAMPLES

### Example 1—Transgenic Expression of Mobile Genome Editing Reagents in Root Stocks

A nucleic acid encoding a CRISPR-Cas nuclease is codon-optimized for expression in soybean. Additional features to further increase nuclease activity include disrupting the protein coding sequence with multiple introns (Grützner et al. Plant Commun. 2021, 2:100135), adding a transcriptional enhancer in the T-DNA of the agrobacterium binary vector (Nuccio et al. Recent Adv. Gene. Expr. Enabling Technol. Crop Plants. Springer New York, 2015:41-77), incorporating a translational enhancer in the 5′-UTR (Gallie and Walbot Nucleic Acids Res 1992, 20:4631-4638), placing a species-specific Kozak sequence at the translation start codon (Kozak Annu Rev Cell Biol 1992, 8:197-225), flanking the coding sequence with optimal nuclear localization signals (Lyck et al. Planta 1997, 202:117-125; Kosugi et al. J Biol Chem 2009, 284:478-485) and utilization of promoters that are highly active in root tissue (Khandal et al. Plant Biotechnol J 2020, 18:2225-2240; Xu et al. Plant Biotechnol J 2020, 18:1585-1597; James et al. Front Plant Sci 2022, 13:1009487) or in particular phloem companion cells (Schmidt et al. Front Plant Sci 2019, 10:1666). A meristem transport segment, like the 102 bp Arabidopsis FT element (Li et al. J Virol 2009, 83:3540-3548; Jackson and Hong Front Plant Sci 2012, 3:127), is fused to the 3′-UTR just after the translation stop codon and before the transcriptional terminator sequence. There are a variety of meristem transport segments to choose from including those based on tRNA sequence (Zhang et al. Plant Cell 2016, 28:1237-1249) or those derived from genes that produce mobile RNAs (Thieme et al. Nat Plants 2015, 1:1-9). The MTS-tagged CRISPR-Cas nuclease is incorporated into a T-DNA vector.

A meristem transport segment, like the 102 bp Arabidopsis FT element (Li et al. J Virol 2009, 83:3540-3548; Li et al. Sci Rep 2011, 1:73) is fused to the 5′- or 3′-terminus of the companion guide RNA or guide RNA array to the CRISPR Cas nuclease and expressed from a suitable RNA polymerase III promoter (Hassan et al. Trends Plant Sci 2021, 26:1133-1152). This construct is incorporated into the same T-DNA vector that includes the gene encoding the MTS-tagged CRISPR-Cas nucleic acid. The guide RNA or guide RNA array DNA sequence can be expressed from an RNA polymerase II promoter if it is flanked by a hammerhead ribozyme at the 5′-terminus and an HDV ribozyme at the 3′-terminus (Gao and Zhao J Integr Plant Biol 2014, 56:343-349). The meristem transport segment must be situated between the two ribozymes.

The T-DNA can also include a reporter gene such as a fluorescent protein fused to a meristem transport segment, like the 102 bp Arabidopsis FT element (Li et al. J Virol 2009, 83:3540-3548; Li et al. Sci Rep 2011, 1:73) to enable tracking of meristem transport segment function in planta. A guide RNA targeting a non-essential or harmless sequence in the rootstock genome may also be included to assess CRISPR system function and aid in the selection of suitable MTS-tagged CRISPR Cas Editor plant lines. Guide RNA(s) whose action might produce a harmless but visible signal in target gene lines, such as an obvious trichome phenotype (Wang et al. Plant Biotechnol J 2019, 17:1706-1722), can also be linked to the meristem transport segment to enable assessment of CRISPR system function in target plants.

The MTS-tagged CRISPR system is transformed into a suitable line—and transformants are selected based on the presence of the T-DNA, fluorescent protein activity and/or CRISPR system activity. A transgenic plant with a transgene that expresses a CRISPR-Cas nuclease is termed an “Editor”. A transgenic plant with a transgene that expresses a mobile CRISPR-Cas nuclease is termed an “MTS-tagged CRISPR Cas Editor”. The regenerates are recovered and grown to maturity to collect seed. Progeny from ideal regenerants are tested for T-DNA heritability and transgene stability. These lines are propagated as needed.

To edit target germplasm the seed for both the MTS-tagged CRISPR Cas Editor line and the target line(s) are germinated on germination paper or by planting in soil. About 5-7 days later the shoots of the target line(s) are grafted to the roots of the MTS-tagged CRISPR Cas Editor line(s) using standard procedures developed for soybean (Bezdicek et al. Agron J 1972, 64:558-558), monocots like corn and wheat (Reeves et al. Nature 2022, 602:280-286), or the species of interest (Warschefsky et al. Trends Plant Sci 2016, 21:418-437). The grafted shoot is then monitored for evidence of fluorescence if a mobile reporter is present in the MTS-tagged CRISPR Cas Editor line, for phenotypic readout, and/or for the presence of the intended edits in new growth of each grafted target plant. Grafted target scions with the intended edits are self-pollinated or crossed to a suitable parent and grown to maturity. The harvested seed are evaluated for inheritance of the intended edits.

This method enables editing of any germplasm that is graft compatible with the MTS-tagged CRISPR Cas Editor line regardless of its transformability. Edited germplasm produced this way will not inherit the transgenes used to produce the Cas nuclease or the guide RNA of the CRISPR Cas system. An additional benefit is that edits can be rapidly propagated into elite commercial lines simultaneously and in a single generation, greatly reducing the time required to produce marketable material.

### Example 2—Transgenic Expression of Mobile Genome Editing Reagents in Hairy Root Stocks

A T-DNA containing an MTS-tagged CRISPR-Cas nuclease and at least one guide RNA as described in Example 1 is transformed directly into Agrobacterium rhizogenes, which is used to infect a rootstock plant to produce hairy roots (Hao et al. Curr Biochem Eng 2021, 7:31-37; Song et al. Curr Protoc 2021, 1: e195). A variety of soybean cultivars are susceptible and produce transgenic hairy roots. The transgenic hairy roots produce the MTS-tagged Cas nuclease and at least one guide RNA which are transported to the shoot apical meristem to modify the stem cells that give rise to the reproductive structures.

The transformed plants are transferred to soil and grown to maturity. The shoot is monitored for evidence of fluorescence, if a mobile reporter is present in the transformed T-DNA, for phenotypic readout, and/or for the presence of the intended edits in new growth of each transgenic plant. Plants with the intended edits are self-pollinated or crossed to a suitable parent and grown to maturity. The harvested seed are evaluated for inheritance of the intended edits.

### Example 3—Transgenic Expression of a Cas Using a Constitutive Promoter Combined with Delivery of MTS-Tagged Guide RNAs

Multiple heritable edits can be introduced into an Editor line constitutively expressing a CRISPR Cas nuclease. A T-DNA containing a CRISPR-Cas nuclease is designed as in Example 1, but utilizing promoters that are highly active in most plant tissues (Binet et al. Plant Mol Biol 1991, 17:395-407; Christensen and Quail Transgenic Res 1996, 5:213-218; Hernandez-Garcia et al. Plant Cell Rep 2009, 28:837-849; Amack and Antunes Curr Plant Biol 2020, 24:100179).

The T-DNA can also include a reporter gene such as a fluorescent protein (Schnitzler et al. Mar Biotechnol 2008, 10:328-342) to enable assessment of T-DNA function in planta. A guide RNA targeting a non-essential or harmless sequence in the Editor plant genome may also be included to assess CRISPR system function and aid in the selection of suitable Editor plant lines. Guide RNA(s) whose action might produce a harmless but visible signal in target gene lines, such as an obvious trichome phenotype (Wang et al. Plant Biotechnol J 2019, 17:1706-1722) to enable assessment of CRISPR system function in target plants can also be used.

MTS-tagged guide RNAs or guide RNA arrays are produced using in vitro transcription (Huang and Yu Curr Protoc Mol Biol 2013, 102:4.15.1-4.15.14) for application to Editor lines. A meristem transport segment, like the 102 bp Arabidopsis FT element (Li et al. J Virol 2009, 83:3540-3548; Li et al. Sci Rep 2011, 1:73) is fused to the 5′- or 3′-terminus of the companion guide RNA or guide RNA array to the CRISPR Cas nuclease and expressed from a suitable RNA polymerase promoter suitable for runoff in vitro transcription, like the T7, T3 or Sp6 promoter. The guide RNA or guide RNA array DNA sequence can be flanked by a hammerhead ribozyme at the 5′-terminus and an HDV ribozyme at the 3′-terminus (Gao and Zhao J Integr Plant Biol 2014, 56:343-349) to produce a precisely terminated product. The meristem transport segment must be situated between the two ribozyme cleavage sites. The guide RNA can be modified as needed to enhance mobility (Maizel et al. Curr Opin Plant Biol 2020, 57:52-60), stability (Filippova et al. Biochimie 2019, 167:49-60; Rozners J Am Chem Soc 2022, 144:12584-12594) and to enable tracking (Awwad et al. MethodsX 2020, 7:101148) when applied to plants. Guide RNAs produced in vitro can be combined with RNase inhibitors and/or methylated with a m⁵C methyltransferase to reduce degradation prior to application.

### Example 4—Application of the gRNA by RNA Spray

Seed representing suitable Editor lines that constitutively express a CRISPR Cas nuclease are germinated in axenic culture or in soil and grown to the first trifoliate stage. Then one of several RNA spray methods (Rank and Koch Front Plant Sci 2021, 12:755203; Dalakouras et al. Front Plant Sci 2016, 7:1327) is used to introduce the MTS-tagged guide RNA(s) to the plant. These include formulations consisting of carbon nanodots (Doyle et al. BioRxiv, 2019:805036), therapeutic nanoparticles (Karny et al. Sci Rep 2018, 8:7589), clay nanosheets (Mitter et al. Nat Plants 2017, 3:1-10), encapsulation (Islam et al. Microb Biotechnol 2021, 14:1847-1856) and surfactants (U.S. Patent No. U.S. Pat. No. 9,121,022B2). A formulation consisting of about 50 μM of each MTS-tagged guide RNA or guide RNA array is prepared and sprayed onto the Editor line. The spray volume should be sufficient to visibly wet the leaf surface. Plants are monitored for guide RNA uptake and mobility using a fluorescent label or a phenotypic readout in new growth post application. New growth is assayed for the presence of the intended edits using any acceptable method including T7E1/TIDE and/or amplicon sequence analysis (Bernabé-Orts et al. Plant Biotechnol J 2019, 17:1971-1984; Lee et al. Plant Biotechnol J 2019, 17:362-372). The MTS-tagged guide RNA treatment can be repeated as needed to produce the desired result. Plants with the intended edits are grown to maturity and the progeny are evaluated for inheritance of the intended edits. Progeny that contain edits are retained. These progeny will not inherit the editing transgenes.

### Example 5—Application of the gRNA by Injection

Seeds representing suitable Editor lines that constitutively express a CRISPR Cas nuclease are germinated in axenic culture or in soil and grown to the first trifoliate stage. An approximately 50 μM solution of each MTS-tagged guide RNA or guide RNA array is prepared in nuclease-free water or phosphate buffer and 1-5 μL is injected in the stem of each Editor seedling, with the injection point being 3-5 cm below the top of the plant. Plants are monitored for guide RNA uptake and mobility using a fluorescent label or a phenotypic readout in new growth, toward the plant apex, post application. New growth is assayed for the presence of the intended edits using any acceptable method including T7E1/TIDE and/or amplicon sequence analysis (Bernabé-Orts et al. Plant Biotechnol J 2019, 17:1971-1984; Lee et al. Plant Biotechnol J 2019, 17:362-372). The MTS-tagged guide RNA treatment can be repeated as needed to produce the desired result. Plants with the intended edits are grown to maturity and the progeny are evaluated for inheritance of the intended edits. Progeny that contain edits are retained. Progeny that inherit the edit but not the transgenes are selected.

### Example 6—Application of the gRNA by Wounding

Seeds representing suitable Editor lines that constitutively express a CRISPR Cas nuclease are germinated in axenic culture or in soil and grown to the first trifoliate stage. An approximately 50 μM solution of each MTS-tagged guide RNA or guide RNA array is prepared in nuclease-free water or phosphate buffer, with or without a wetting agent such as Silwet-77. The surface of the first expanded leaf is gently wounded using an abrasive agent such as glass beads or 400 grit sandpaper and 1-5 μL of the guide RNA solution is applied to the wound site. Plants are monitored for guide RNA uptake and mobility using a fluorescent label or a phenotypic readout in new growth, toward the plant apex, post application. New growth is assayed for the presence of the intended edits using any acceptable method including T7E1/TIDE and/or amplicon sequence analysis (Bernabé-Orts et al. Plant Biotechnol J 2019, 17:1971-1984; Lee et al. Plant Biotechnol J 2019, 17:362-372). The MTS-tagged guide RNA treatment can be repeated as needed to produce the desired result. Plants with the intended edits are grown to maturity and the progeny are evaluated for inheritance of the intended edits. Progeny that contain edits are retained. Progeny that inherit the edit but not the transgenes are selected.

### Example 7—Application of the gRNA by Bathing

Seeds representing suitable Editor lines that constitutively express a CRISPR Cas nuclease are germinated on germination paper for 1-3 days. An approximately 50 μM solution of each MTS-tagged guide RNA or guide RNA array is prepared in nuclease-free water or phosphate buffer, with or without a wetting agent like Silwet-77. Each seedling is placed in the MTS-tagged guide RNA solution and incubated overnight in a humid chamber. The treated seedlings are then transferred to soil. Plants are monitored for guide RNA uptake and mobility using a fluorescent label or a phenotypic readout in new growth, toward the plant apex, post application. New growth is assayed for the presence of the intended edits using any acceptable method including T7E1/TIDE and/or amplicon sequence analysis (Bernabé-Orts et al. Plant Biotechnol J 2019, 17:1971-1984; Lee et al. Plant Biotechnol J 2019, 17:362-372). The MTS-tagged guide RNA treatment can be repeated as needed to produce the desired result. Plants with the intended edits are grown to maturity and the progeny are evaluated for inheritance of the intended edits. Progeny that contain edits are retained. Progeny that inherit the edit but not the transgenes are selected.

### Example 8—Transgenic Expression of MTS-Tagged Cas Nuclease in Rootstock, Enabling Editing in Elite Germplasm by Grafting Target Shoots to Transgenic Root Stock

Multiple heritable edits can be introduced into an Editor rootstock line constitutively expressing an MTS-tagged CRISPR Cas nuclease. A T-DNA containing a CRISPR-Cas nuclease is designed and produced as in Example 3, but with an MTS-tagged Cas nuclease. An MTS, like the 102 bp Arabidopsis FT element (Li et al. J Virol 2009, 83:3540-3548; Jackson and Hong Front Plant Sci 2012, 3:127), is fused to the 3′-UTR just after the translation stop codon and before the transcriptional terminator sequence. There are a variety of meristem transport segments to choose from including those based on tRNA sequence (Zhang et al. Plant Cell 2016, 28:1237-1249) or derived from genes that produce phloem mobile RNAs (Thieme et al. Nat Plants 2015, 1:1-9).

The T-DNA can also include a reporter gene such as a fluorescent protein (Schnitzler et al. Mar Biotechnol 2008, 10:328-342) fused to an MTS, like the 102 bp Arabidopsis FT element (Li et al. J Virol 2009, 83:3540-3548; Li et al. Sci Rep 2011, 1:73) to enable tracking of meristem transport segment function in planta. A guide RNA targeting a non-essential or harmless sequence in the editor plant genome may also be included to assess CRISPR system function and aid in the selection of suitable MTS-tagged CRISPR Cas Editor plant lines. Guide RNA(s) whose action might produce a harmless but visible signal in target gene lines, such as an obvious trichome phenotype (Wang et al. Plant Biotechnol J 2019, 17:1706-1722), can also be linked to the MTS to enable assessment of CRISPR system function in target plants.

The MTS-tagged CRISPR system is transformed into a suitable line and transformants are selected based on the presence of the T-DNA, fluorescent protein activity, and/or CRISPR system activity. The ideal MTS-tagged CRISPR Cas Editor line has a high fluorescent protein signal and a highly active CRISPR system based on analysis of the harmless/non-essential target site using any suitable tool including T7E1/TIDE and/or amplicon sequencing (Bernabé-Orts et al. Plant Biotechnol J 2019, 17:1971-1984; Lee et al. Plant Biotechnol J 2019, 17:362-372). T-DNA copy number is a secondary criterium to robust, stable CRISPR system activity in healthy regenerants. The regenerates are recovered and grown to maturity to collect seed. Progeny from ideal regenerants are tested for T-DNA heritability and transgene stability. These lines are propagated as needed.

To edit target germplasm the seed for both the MTS-tagged CRISPR Cas Editor line and the target line(s) are germinated on germination paper or by planting in soil. About 5-7 days later the shoots of target line(s) are grafted to the roots of the MTS-tagged CRISPR Cas Editor line(s) using standard procedures developed for soybean (Bezdicek et al. Agron J 1972, 64:558-558), monocots like corn and wheat (Reeves et al. Nature 2022, 602:280-286), or the species of interest (Warschefsky et al. Trends Plant Sci 2016, 21:418-437). The grafted shoot is then monitored for evidence of fluorescence (if a mobile reporter is present in the MTS-tagged CRISPR Cas Editor line), phenotypic readout and/or the presence of the intended edits in new growth of each grafted plant.

MTS-tagged guide RNAs or guide RNA arrays are produced using in vitro transcription (Huang and Yu Curr Protoc Mol Biol 2013, 102:4.15.1-4.15.14) for application to the MTS-tagged CRISPR Cas nuclease Editor lines. A meristem transport segment, like the 102 bp Arabidopsis FT element (Li et al. J Virol 2009, 83:3540-3548; Li et al. Sci Rep 2011, 1:73) is fused to the 5′- or 3′-terminus of the companion guide RNA or guide RNA array to the CRISPR Cas nuclease and expressed from an RNA polymerase promoter suitable for runoff in vitro transcription, like the T7, T3 or Sp6 promoter. The guide RNA or guide RNA array DNA sequence can be flanked by a hammerhead ribozyme at the 5′-terminus and an HDV ribozyme at the 3′-terminus (Gao and Zhao J Integr Plant Biol 2014, 56:343-349) to produce a precisely terminated product. The meristem transport segment must be situated between the two ribozyme cleavage sites. The guide RNA can be modified as needed to enhance mobility (Maizel et al. Curr Opin Plant Biol 2020, 57:52-60), stability (Filippova et al. Biochimie 2019, 167:49-60; Rozners J Am Chem Soc 2022, 144:12584-12594) and to enable tracking (Awwad et al. MethodsX 2020, 7:101148) when applied to plants.

Suitable grafted MTS-tagged CRISPR Cas nuclease Editor lines are grown to the first trifoliate stage. The method of any of Examples 4-7 is used to introduce MTS-tagged gRNA(s) to the plant.

1. A method of editing a genomic target in a scion comprising grafting the scion onto a rootstock comprising nucleic acid encoding a Cas9 nickase or Cas12 nuclease and nucleic acid encoding a guide RNA for the Cas9 nickase or Cas12 nuclease, wherein the nucleic acid encoding the guide RNA and the nucleic acid encoding the Cas9 nickase or the Cas12 nuclease are fused to nucleic acid encoding a meristem transport segment (MTS), wherein the genome of a cell in a meristem of the scion is edited.

2. The method of claim 1 wherein the Cas9 nickase or Cas12 nuclease is associated with a reverse transcriptase or fused to a reverse transcriptase, and wherein the guide RNA comprises at its 3′ end a priming site and an edit to be incorporated into the genomic target.

3. (canceled)

4. (canceled)

5. The method of claim 1, wherein the Cas12 nuclease is a Cas12 nickase.

6-10. (canceled)

11. The method of claim 1, wherein the nucleic acid encoding the Cas9 nickase or Cas12 nuclease and the nucleic acid encoding the guide RNA are provided to the rootstock in the same vector.

12. The method of claim 1, wherein the nucleic acid encoding the Cas9 nickase or Cas12 nuclease and the nucleic acid encoding the guide RNA are provided to the rootstock in different vectors.

13. (canceled)

14. The method of claim 1, wherein the scion and the rootstock are different plant species.

15. The method of claim 1, wherein the scion and the rootstock are the same plant species.

16. The method claim 1, wherein the scion and/or rootstock is a dicot.

17. The method claim 1, wherein the scion and/or rootstock is a monocot.

18. The method of claim 1, wherein the scion is soy, canola, alfalfa, corn, oat, sorghum, sugarcane, banana or wheat.

19. The method of claim 1, wherein the MTS comprises:
i. a meristem transport component (MTC);
ii. an RNA hairpin comprising a first stem of 8 to 12 nucleotides, at least one variable bulge, a second stem of 4 to 7 nucleotides, and a variable loop;
iii. a Flowering Locus T (FT-) derived sequence, wherein the FT-derived sequence comprises the nucleotide sequence set forth in SEQ ID NO: 5, 6, 7, 8, 9, 10, 11, 12, 13, 14, 15, 16, 17, 18, 19, 20, 21, 22, 23, or 24; or
iv. a tRNA-like sequence (TLS), wherein the TLS comprises the nucleotide sequence set forth in SEQ ID NO: 29 or 30.

20. (canceled)

21. (canceled)

22. The method of claim 1, wherein the nucleic acid encoding the MTS is located 3′ of the nucleic acid encoding the Cas9 nickase or Cas12 nuclease and/or 3′ of the nucleic acid encoding the guide RNA.

23. The method of claim 1, wherein the nucleic acid encoding the MTS is located 5′ of the nucleic acid encoding the Cas9 nickase or Cas12 nuclease and/or 5′ of the nucleic acid encoding the guide RNA.

24. The method of claim 1, wherein the nucleic acid encoding the Cas9 nickase or Cas12 nuclease is operably linked to a promoter, wherein the promoter is active in roots and/or phloem companion cells.

25. (canceled)

26. The method of claim 24, wherein:
the promoter is the promoter of a gene selected from the group consisting of Arabidopsis WRKY6, chickpea WRKY31, carrot MYB113, corn GLU1, strawberry RB7-type TIP-2, and banana TIP2-2, or the promoter of an orthologous gene thereof.

27. The method of claim 24, wherein the promoter is selected from the group consisting of a promoter from a Flowering Locus T (FT) gene, a promoter from a Fabaceaen FOR 1 gene, a rice tungro bacilliform virus promoter, an RmlC-like cupins superfamily protein promoter, a Commelina yellow mottle virus promoter, a wheat dwarf virus promoter, a sucrose synthase promoter, a glutamine synthetase promoter, a phloem-specific isoform of plasmamembrane H+-ATPase promoter, a JMJ18 promoter, and a phloem protein 2 (PP2) promoter.

28.-30. (canceled)

31. The method of claim 1, wherein the nucleic acid encoding the guide RNA is operably linked to a promoter.

32. The method of claim 31, wherein the promoter is an RNA polymerase II promoter or an RNA polymerase III promoter.

33. (canceled)

34. The method of claim 1, wherein the nucleic acid encoding the guide RNA and the MTS is located between two ribozyme sequences.

35. (canceled)

36. The method of claim 1, wherein the nucleic acid encoding the guide RNA and the MTS further comprises a hammerhead ribozyme sequence 5′ to the nucleic acid encoding the guide RNA and the MTS, and an HDV ribozyme 3′ to the nucleic acid encoding the guide RNA and the MTS.

37. (canceled)

38. (canceled)

39. The method of claim 1, wherein the rootstock comprises nucleic acid encoding two, three, four, five, or more than five guide RNAs.

40. (canceled)

41. The method of claim 1, wherein the Cas12 nuclease is selected from the group consisting of Cas12a (Cpf1), Cas12e (CasX), Cas12d (CasY), Cas12h, Cas12i, and Cas12j.

42. (canceled)

43. The method of claim 1, further comprising retrieving a progeny of the scion, wherein the progeny has an altered genome.

44. The method of claim 1, wherein two or more guide RNAs are encoded by a single precursor RNA, wherein the two or more guide RNAs are each flanked by a direct repeat.

45. (canceled)

46. An edited plant produced by the method of claim 1.

47. A rootstock comprising:
nucleic acid encoding a Cas9 nickase or Cas12 nuclease, and
nucleic acid encoding a guide RNA for the Cas9 nickase or Cas12 nuclease,
wherein the nucleic acid encoding the guide RNA and the nucleic acid encoding the Cas9 nickase or Cas12 nuclease are fused to nucleic acid encoding a meristem transport segment (MTS).

48.-72. (canceled)


SEQUENCE LISTING (62 sequences)

SEQ ID NO: 1 - DNA, 103 bp, Arabidopsis thaliana
  source 1..103: mol_type = unassigned DNA
atgtctataaatataagagaccctcttatagtaagcagagttgttggagacgttcttgatccgtttaatagatcaatcactctaaaggttacttatggccaaa

SEQ ID NO: 2 - DNA, 975 bp, Arabidopsis thaliana
  source 1..975: mol_type = unassigned DNA
agttaatgcaaatccgaaacagtataaatatgtgtagagggttcatgcctatgatacaaattaaagaagcagaaacaaaaacaagtaaaacagaaacaatcaacacagagaaaccacctgtttgttcaagatcaaagatgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttagatggcttcttcctttataaccaattgatattgcatactctgatgagatttatgcatctatagtattttaatttaataaccattttatgatacgagtaacgaacggtgatgatgcctatagtagttcaatatataagtgtgtaataaaaatgagagggggaggaaaatgagagtgttttacttatatagtgtgtgatgcgataattatattaatctacatgaaatgaagtgttatatttatactttacgtgtattcatttcttttcgatgcaaaaatcaggcagtgggaagaatctgctgttttacttttg

SEQ ID NO: 3 - DNA, 762 bp, Zea mays
  source 1..762: mol_type = unassigned DNA
ttgagagttctaataagagcaacggccaataccattagcgagttatttttctgcaatatatgtcagcaaccgatcatttggttatggctcgtgtcatacaggatgtattggatccctttacaccaaccattccactaagaataacgtacaacaataggctacttctgccaagtgctgagctaaagccatccgcggttgtaagtaaaccacgagtcgatatcggtggcagtgacatgagggctttctacaccctggtactgattgacccggatgccccaagtccaagccatccatcactaagggagtacttgcactggatggtgacagatattccagaaacaactagtgtcaactttggccaagagctaatattttatgagaggccggacccaagatctggcatccacaggctggtatttgtgctgttccgtcaacttggcagggggacagtttttgcaccagaaatgcgccacaacttcaactgcagaagctttgcacggcaatatcacctcagcattgccaccgctacacatttcaactgtcaaagggaaggtggatccggcggaagaaggtttagggaagagtagaaaccataggccactgcatggtcacactatagaaatatcatcaataatgtgcactatattgaatcaatgcaccacctctatatgctgaatgttatgtatctcaaactatgattgtactgacttgaaaggttgagagcttagtctcttagcagaatatagcacaatattactagta

SEQ ID NO: 4 - DNA, 531 bp, Glycine max
  source 1..531: mol_type = unassigned DNA
atgcctagtggaagtagggatcctctcgttgttgggggagtaattggggatgtattggatccttttgaatattctattcctatgagggttacctacaataacagagatgtcagcaatggatgtgaattcaaaccctcacaagttgtcaaccaaccaagggtaaatatcggtggtgatgacctcaggaacttctatactttgattgcggttgatcccgatgcacctagcccaagtgaccccaatttgagagaatacctccattggttggtgactgatatcccagcaacaacaggggctagtttcggccatgaggttgtaacatatgaaagtccaagaccaatgatggggattcatcgtttggtgtttgtgttatttcgtcaactgggtagggagaccgtgtatgcaccaggatggcgccagaatttcaacactaaagaatttgctgaactttacaaccttggattgccagttgctgctgtctatttcaacattcagagggaatctggttctggtggaaggaggttatactaa

SEQ ID NO: 5 - RNA, 103 nt, Arabidopsis thaliana
  source 1..103: mol_type = unassigned RNA
atgtctataaatataagagaccctcttatagtaagcagagttgttggagacgttcttgatccgtttaatagatcaatcactctaaaggttacttatggccaaa

SEQ ID NO: 6 - RNA, 975 nt, Arabidopsis thaliana
  source 1..975: mol_type = unassigned RNA
agttaatgcaaatccgaaacagtataaatatgtgtagagggttcatgcctatgatacaaattaaagaagcagaaacaaaaacaagtaaaacagaaacaatcaacacagagaaaccacctgtttgttcaagatcaaagatgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttagatggcttcttcctttataaccaattgatattgcatactctgatgagatttatgcatctatagtattttaatttaataaccattttatgatacgagtaacgaacggtgatgatgcctatagtagttcaatatataagtgtgtaataaaaatgagagggggaggaaaatgagagtgttttacttatatagtgtgtgatgcgataattatattaatctacatgaaatgaagtgttatatttatactttacgtgtattcatttcttttcgatgcaaaaatcaggcagtgggaagaatctgctgttttacttttg

SEQ ID NO: 7 - RNA, 762 nt, Zea mays
  source 1..762: mol_type = unassigned RNA
ttgagagttctaataagagcaacggccaataccattagcgagttatttttctgcaatatatgtcagcaaccgatcatttggttatggctcgtgtcatacaggatgtattggatccctttacaccaaccattccactaagaataacgtacaacaataggctacttctgccaagtgctgagctaaagccatccgcggttgtaagtaaaccacgagtcgatatcggtggcagtgacatgagggctttctacaccctggtactgattgacccggatgccccaagtccaagccatccatcactaagggagtacttgcactggatggtgacagatattccagaaacaactagtgtcaactttggccaagagctaatattttatgagaggccggacccaagatctggcatccacaggctggtatttgtgctgttccgtcaacttggcagggggacagtttttgcaccagaaatgcgccacaacttcaactgcagaagctttgcacggcaatatcacctcagcattgccaccgctacacatttcaactgtcaaagggaaggtggatccggcggaagaaggtttagggaagagtagaaaccataggccactgcatggtcacactatagaaatatcatcaataatgtgcactatattgaatcaatgcaccacctctatatgctgaatgttatgtatctcaaactatgattgtactgacttgaaaggttgagagcttagtctcttagcagaatatagcacaatattactagta

SEQ ID NO: 8 - RNA, 531 nt, Glycine max
  source 1..531: mol_type = unassigned RNA
atgcctagtggaagtagggatcctctcgttgttgggggagtaattggggatgtattggatccttttgaatattctattcctatgagggttacctacaataacagagatgtcagcaatggatgtgaattcaaaccctcacaagttgtcaaccaaccaagggtaaatatcggtggtgatgacctcaggaacttctatactttgattgcggttgatcccgatgcacctagcccaagtgaccccaatttgagagaatacctccattggttggtgactgatatcccagcaacaacaggggctagtttcggccatgaggttgtaacatatgaaagtccaagaccaatgatggggattcatcgtttggtgtttgtgttatttcgtcaactgggtagggagaccgtgtatgcaccaggatggcgccagaatttcaacactaaagaatttgctgaactttacaaccttggattgccagttgctgctgtctatttcaacattcagagggaatctggttctggtggaaggaggttatactaa

SEQ ID NO: 9 - RNA, 699 nt, Cucurbita maxima
  source 1..699: mol_type = mRNA
ggggcttcaaaagagaattaggtcacctcccagctcggtttcgacacgccatgccgagaaatcgtgaccctctagtcgtcgggagagtgatcggcgacgtcgtcgactcgttctcgaggtccatctcgattagggttgtttacgactcgagggaagttaacaatgggtgtgagctcaaaccctctcaagctgtcaacaagccaagagttgagattggtggcactgaccttcgcaccttcttcactttggttatggtggatcccgacgctcctagccctagcgatcccaatctaagagaatacttgcattggttagtgaccgatattccagctacaaccgaggcaacctttggacaagagatagtgtgctacgagaatccaagaccaacggtgggtatccaccgttttgtgctggtcttgttccggcagctcggaaggcaaacggtgtatgctcctgggtggcgccagaacttcaacaccagacactttgcagagctttacaatcttggttcgccagtcgccgccgtctatttcaattgccaaagggaaaatggctccggtggaaggagaagagccggcgatgaatgttcataaaaacacttcacttcacattatattatcaaccaatatattgtaataacatggttcacgtttctatctaatagattatatatttttaataagttcgtgaaaaaaaaaa

SEQ ID NO: 10 - RNA, 858 nt, Cucurbita maxima
  source 1..858: mol_type = mRNA
gagacaattacgcatcttttcagctctctcacgtactaccatcctctcgacgccatgccgagagaccgtgaccctttggtcgttgggagagtcatcggcgacgttatcgactcgttcacgaagtccatttcgattagggctacttacaacaacagggaaattagcaatggctgtgagctcaaaccctctcaagttgtcaaccagccaagagttgagattggtggcactgaccttcgcaccttcttcactttggttatggtggatcctgatgctcctagccctagtgatcctaatctaagggaatacttgcattggttggtgactgatatcccagctacaactggagcgaactttggtcaagagatcgtgtgctatgagagcccaagacccacggtgggtatccatcgtcttgtgctggtgttgtttcgacagcttggaaggcaaacggtgtacgctcctgggtggcgccagaacttcaacacaagagactttgcagagctttacaatcttggcttgccggtggcagccgtttatttcaattgccaaagggaaagtgggtctggtggaaggagaagaacccaagatgatttctaagccccacttcacattaattagattaatattatagcccctatcatctattaatcctaccttgcttttagattaacctttattttgagtacacccatggatcataaataagcccaaaatgcattcctaatattgctcttatactcgtttcgtatgaatcactgtcttttcttctttgtttttcttgttcgagtgttcatgttgtgctttttttttcgtatgaatcaaagtagaagatcaagattcgaaaaaaaaaaaaaaaaaaaa

SEQ ID NO: 11 - RNA, 596 nt, Vitis vinifera
  source 1..596: mol_type = mRNA
ccctcttgtattgtatcggtgaggtgtgtgtgatgcctagggaaagggatcctcttgttgttgggcgcgttgtcggggatgttctggacccctttctcaggtccatcactctgagggtgacctacaataatagagaagtagcaaatggctgtgagttcagaccctctcagctagtcagccaacctagggtggacattggaggggatgacttgaggaccttctatactttggttatggtggaccctgacgctccaagccccagtaatccgaacctaagggagtacttacattggttggtgactgatattccagcaactactggggcaaacttcggccaagagattgtgtgttatgagagcccacgcccaacagctgggattcatcgctttgtttttgtattgtttcgccaactgggtaggcagacagtgtatgcaccagggtggcgccaaaatttcaacactagggactttgctgagctttataatcttggtttgcctgttgctgctgtttattttaactgccaaagggagggcggctcgggtggtcgaagatcataatcaatggattttgtacgcaaccttgcgacttacaaaggc

SEQ ID NO: 12 - RNA, 525 nt, Malus x domestica
  source 1..525: mol_type = mRNA
atgcctagggatagggacccccttgttgttggacgagtggttggtgatgttttagaccccttcacaaggtctgtttctctgagggtgacctacggtactaaggaggttaacaatggttgtgagctcaaaccttctgaagttgtccaacaacctagagctgatattggtggagacgatctcaggactttctacactctggtcatggtggatcctgatgcacccagcccaagtgaccccaacctaaaggaatatttgcattggttggttaccgatattccagcaactactgcggcaagcttcgggcaagagatcgtgtgttatgaaagtccacggccaacagtggggattcatcgctttgttttggtggtgtttcgccaattgggtaggcaaacggtgtatgctccgggatggcgccagaacttcaataccagagacttcgccgagctttataatcttggattaccggtgtctgtcgtctattttaactgccaaagggagggcggctccggtggaaggagaagataa

SEQ ID NO: 13 - RNA, 745 nt, Citrus unshiu
  source 1..745: mol_type = mRNA
ggcacgaggaatagtcttactacttttgtaggctgtgtgtgtatttgtttgtgcttagtgttgttgagtgtttgtttgtgtttagtgttgttgatatgtctagcagggagagagatcctcttattgttggccgcgttgttggtgatgttcttgacaattttacaagaacaattccaatgaggattacctattcaaacaaggatgttaataatggccgtgagctcaaaccttctgaagttctgaaccagcctagggctgaaattggtggtgatgatcttaggacattttatactttggtaatggttgatcctgatgcaccaagcccaagtgaccccagccttagggagtatttgcattggttggtgactgatattccagcaaccacaggggccagctttggccaagagattgtgaactatgaaagccctaggccaacgatggggattcacaggtttgtctttgtgttgttccggcaacttgggaggcagactgtttatgcaccagggtggcgtcagaacttcagcacgagggattttgctgagctttacaatctgggacctccggtggccgctgtctacttcaactgccagagggagagcggatccggcggaaggcctgtcagacgatgatccatacatgcttaatttgatatcaaattacacacacacacacacacacacacacacacacacacacacacacacacacacactatttatatatatatatatatatatatatatat

SEQ ID NO: 14 - RNA, 534 nt, Lycopersicon esculentum
  source 1..534: mol_type = unassigned RNA
atgcctagagaacgtgatcctcttgttgttggtcgtgtggtaggggatgtattggaccctttcacaagaactattggcctaagagttatatatagagatagagaagttaataatggatgcgagcttaggccttcccaagttattaaccagccaagggttgaagttggaggagatgacctacgtacctttttcactttggttatggtggaccctgatgctccaagtccgagtgatccaaatctgagagaataccttcactggttggtcaccgatattccagctaccacaggttcaagttttgggcaagaaatagtgagctatgaaagtccaagaccatcaatgggaatacatcgatttgtatttgtattattcagacaattaggtcggcaaacagtgtatgctccaggatggcgtcagaatttcaacacaagagattttgcagaactttataatcttggtttacctgttgctgctgtctattttaattgtcaaagagagagtggcagtggtggacgtagaagatctgctgattga

SEQ ID NO: 15 - RNA, 525 nt, Populus tremula
  source 1..525: mol_type = mRNA
atgtcaagggacagagatcctctgagcgttggccgtgttataggggacgtgctggaccccttcacaaagtctatcccgctcagggtcacctacaactccagagaggtcaacaatggttgcgagctcaaaccctctcaggttgccaaccagccgagggttgatattggcggggaagatctaaggaccttctacactctggttatggtggaccctgatgcacccagcccaagtgaccccagcctcagagaatatttgcattggttggtgactgatattccagcaacaacgggggcaagctttggccatgaaactgtgtgctatgagagcccgaggccgacgatggggattcatcggtttgttttcgtcttgttccggcaactgggcaggcaaactgtgtatgcccctgggtggcgccagaacttcaacaccagagactttgctgaggtctacaatcttggatcgccggtggctgctgtttatttcaactgccagagggagagtggctctggtggtaggaggcgataa

SEQ ID NO: 16 - RNA, 534 nt, Hordeum vulgare
  source 1..534: mol_type = unassigned RNA; sub_species = vulgare
atggccgggagggacagggatccgctggttgtcggcagggttgtgggggacgtgctggaccccttcgtccgaaccaccaacctcagggtgaccttcgggaacagggccgtgtccaacggctgcgagctcaagccgtccatggtcgcccagcagccgagggtggaggtgggcggcaatgagatgaggaccttctacacgctcgtgatggtagacccagatgctccaagtcctagcgaccccaaccttagagagtatctccactggttggtgacagatatcccgggtacaactggggcgtcgttcgggcaggaggtgatgtgctacgagagccctcgtccaaccatggggatccaccgcttcgtgctcgtgctcttccagcagctggggcggcagacggtgtacgcccccgggtggcgccagaacttcaacaccagggactttgccgagctctacaacctcggccagcccgttgccgccgtctacttcaactgccagcgcgaggccggctccggcggcaggaggatgtacaattga

SEQ ID NO: 17 - RNA, 537 nt, Hordeum vulgare
  source 1..537: mol_type = unassigned RNA; sub_species = vulgare
atggtggggagcagcatgcagcgcggggacccgctggtggtggggcgggtgatcggcgacgtggtggacccgttcgtgcggcgggtggcgctgcgggtcggctacgcgtccagggacgtggccaacggctgcgagctccggccgtccgccatcgccgaccagccgcgcgtcgaggtcggcggcccggacatgcgcaccttctacaccctggtgatggtggatccggatgctccaagccccagcgaccccagccttagggagtacttgcactggctggtcaccgacatcccggccacgacaggagtgtcttttggtaccgaggttgtgtgctacgagggcccgcggccggtgctcgggatccaccgactggtgttcctgctcttccagcaactcggccgacagacggtgtacgccccggggtggcggcagaacttcagcacccgcgactttgccgagctctacaacctcggcctgcccgtcgccgccgtctacttcaactgccagagggagaccggaaccggcgggagaaggatgtga

SEQ ID NO: 18 - RNA, 847 nt, Oryza sativa
  source 1..847: cultivar = Nipponbare; mol_type = mRNA; note = Japonica Group
tgcaccacacacagttcagctagcagatcacctagctagatagctgcctctatcacagtatatttgctccctgcaacttgctgctgctgcaatagctagcagctgcagctagtaagcaaaactataaaccttcagggttttttgcaagatcgatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggcaggctccggcggcaggagggtctacccctagctaacgatgatcccgatcgatctgctgcatgctcactatcatcatccagcatgctatacattgcaggttcagacaattgaaatgattctcgacacacaacatatatatgatggtgtaattaattatgcaattaaatagctgagcaaggctaaggt

SEQ ID NO: 19 - RNA, 866 nt, Oryza sativa
  source 1..866: mol_type = mRNA; note = Japonica Group
cctgtcactgtttggctagcttaaccttcctgacatctatcctctggattgaacggcaggagatacctaagctagctagcaatctctatcgatctgtttgtttacatgttcagttaaaggttactgagaaatgcctagagtttttccggctagcttcataagttagtgggttagctgacctagattcaaagtctaatccttttatttatttgatattagatatcctaacgtttttagttagaggttattaatttgacatggccggcagcggcagggacgatcctcttgtggttggcaggattgtgggtgatgtgctggatccattcgtccggatcactaacctcagtgtcagctatggtgcaaggatcgtctccaatggctgcgagctcaagccgtccatggtgacccaacagcccagggtcgtggtcggtggcaatgacatgaggacgttctacacactcgtgatggtagacccggatgctccgagcccaagcaaccctaaccttagggagtatctacactggctggtcaccgatattcctggtaccactggagcaacatttgggcaagaggtgatgtgctacgagagcccaaggccaaccatggggatccaccggctggtgttcgtgctgttccagcagctggggcgtcagacggtgtacgcaccggggtggcgccagaacttcagcaccaggaacttcgccgagctctacaacctcggctcgccggtcgccaccgtctacttcaactgccagcgcgaggccggctccggcggcaggagggtctacccctagctagctacgcatgccacccggcctccatgcatgcagcagctatagctaagctgagacctgcctagctgtata

SEQ ID NO: 20 - RNA, 848 nt, Ipomoea nil
  source 1..848: mol_type = mRNA
cacacacacacacatatatatacagagaaaggttagttttgatcgaggagctgagctagctaggatgcgaaggggaacagtagaccctttggtgttggggcgtgtgatcggagacgttgtggatccattcacgaggtccgttgagcttagggtggtttacaataacgaggtggatatcaggaatgggtgtgagatgaggccttctcagctcatcaacccacctagggttgaaatcggcggacacgatctccgtactttctacactctggttatggtggatcctgatgctccaagtccaacctctccaaccctgagggaatacctccactggttggtcactgatataccaggaactacaggagcaagcttcggcaatgaagcgatattctacgagcctccaaggccgtcaatgggaatccaccgttttgtgtttgtgcttttccggcaacttggccggcagacagtttatgcaccggtttggcgccagaatttcaacactcgaaactttgctgagatttacaatcttggtttgccagtggccgtcacttactttaacggccaaagggagggtggcaccggcggtcgatctccggcagagccctgggcagccgattaattaccctgctccttcccgttaatttcatgcatgcatgcatgctatctatagcataacatacatatagtatatatcataaataaataagaccacatgcattaacatgtttaattttcccatgaatatatgttaaagttgttctagaagaactacgtactccattatattaccctttatatatggcaatgaagatggtttcatctctatttagaagctaaaaaaaaaaaaaaaa

SEQ ID NO: 21 - RNA, 798 nt, Arabidopsis thaliana
  source 1..798: mol_type = mRNA
tttattgagatacttgagatccaagataaatatgtctttagtcgtagagatcctcttgtggtcggcagtgttgttggagatgttcttgatcctttcacgaggttggtctctcttaaggtcacttatggccatagagaggttactaatggcttggatctaaggccttctcaagttctgaacaaaccaatagtggagattggaggagacgacttcagaaatttctacaccttggttatggtggatccagatgtgccgagtccaagcaaccctcaccaacgagaatatctccactggttggtgactgatatacctgccaccactggaaatgcctttggcaatgaggtggtgtgctacgagagtccacgtcccccctcgggaattcatcgtattgtgttggtattgttccggcaactcggaagacaaacggtttatgcaccggggtggcgccaacagttcaacactcgtgagtttgctgagatctacaatcttggtcttcctgtggctgcctcttacttcaactgccagagggagaatggctgtgggggaagaagaacgtagatgcgtacctacttacgttaactaataatctaatcgtataatattcccttaatgaagtatttaagcatctatgtcaatgtaataagaatttaaagatacgagctaaaaaaaatgatgcatatgctgacatcgatgtaaagtagtttacacttttaatgtaataactaggttttaacccgcggtacaccgcgagactattttgtttttttaagaataaaaatataatttgtttagtcgatt

SEQ ID NO: 22 - RNA, 519 nt, Glycine max
  source 1..519: cultivar = Toyoharuka; mol_type = unassigned RNA
atggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggttatggtggatgcagatgcacctagccctagcaaccctgtcttgagggaataccttcactggatggtgacagatattccagctaccacaaatgcaagctttgggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 23 - RNA, 901 nt, Zea mays
  source 1..901: mol_type = mRNA
agagcacatccgtagtgtgtgcatgcatcacagtcacacacacacagcagaagaagaagaaaccgaacgagggtttagctagcaaaataaacagaagcaagcaagctagctagagctaaggatcgagatcgagatcgaccgaccgacgacgatcagctagcatggcgcgcttcgtggatccgctggtggtggggcgggtgatcggcgaggtggtggacctgttcgtgccttccatctccatgaccgtcgcctatgatggccccaaggacatcagcaacggctgcctcctcaagccgtccgccaccgccgcgccgccgctcgtccgcatctccggccgccgcaacgacctctacacgctgatcatgacggaccccgatgcgcctagccccagcaacccgaccatgagggagtacctccactggatagtgattaacataccaggaggaacagatgctactaaaggtgaggaggtggtggagtacatgggcccgcggccgccggtgggtatccaccgctacgtgctggtgctgttcgagcagaagacgcgcgtgcacgcggaggcccccggcgaccgcgccaacttcaagacgcgcgcgttcgcggcggcgcacgagctcggcctccccactgccgtcgtctacttcaacgcgcagaaggagcccgccagccgccgccgctagctagcagctcctctctgaggcatgccagatgcatgcgtgtgcgtgcaggtgcaaccaccgcactgccggcggctacgtatgaccggtgaataaaaagttttactgcaccgtaagcatgctcgccctgttgctattggtatatgttagcagtgtggcagtctgtatgtagtagctattcgcttgcatctatgcactctatgttagtatgcgtacgtgtggtt

SEQ ID NO: 24 - RNA, 1069 nt, Zea mays
  source 1..1069: mol_type = mRNA
tggcaaaaacccagcgctttgtgccgccgccgtccgccggcccctctgcccttgtacgcgcacctagacacatcgtcatcgatcatcacacgcaatcgacacaagaagttaataaacagcccaaggacgcagagatcagctgatcgagaaggacttgtactactactcagtattgtcgtcacatgcacatatatgtacataaagagctagctacctgagctctacccaaggtcgcgttgatcgatcgatcatggcgcggttcgtggacccgctggtggtggggcgggtgatcggcgaggtggtggacctgttcgtgccctccgtctccatgaccgtcgcctatggccccaaagacatcagcaacggctgcctcctcaagccgtccgccaccgccgcgccgccgctcgtccgcatctccggccgccgcgacgacctctacacgctgatcatgacggacccagatgcgcctagccccagcgacccgaccatgagggagtacctccactggatagtgactaacataccaggaggaacggatgcaaacaaaggtgaggaggtggtggagtacatgggcccgcggccgccggtcggaatccaccgctacgtgctggtgctgttcgagcagaagacgcgtgtgcacgcggagggtcccggtgagcgcgccaacttcaacacacgcgcgttcgcggcggcgcacgagctcggcctccccaccgccgtcgtgtacttcaacgcgcagaaagagccggccaaccaccgccgccgctagctagtagctccaacaagggcgcgccagctgagctgcgtgcgtgcaacccaccacacagccgccggcgaaggctgcctatatgaccggcgaataaaaagtcttactgcaccgtccgtaagcgtactctctgttggtatatgcttgtcttcaggctcttgagtctatctacttaaatgtggttaccactgagtaatagaagcagttggcgcttcgatcgatcattctaatatccgtacgtgtcaatcattcctgtttccatcatcttgcatttgaagacgcattggttctacaccaaggtgt

SEQ ID NO: 25 - RNA, 1288 nt, Cucurbita maxima
  source 1..1288: mol_type = mRNA
gactttttattcaacaatctctctctctctctctcaacttccgatcaagtctctccgccgtcttttcaccggagctgacaattccgatcattttttgcttcccttaaatttccggcatggaggaaccaccgccaaacgccttggatttgccccctggcttcagattccaccccaccgacgaggagatcgtcacttattacctgatacataagatcaccgacgccgccttcactgccaccgccatcggagaagctgacctgaataagtgtgaaccttgggatttgccacataaagctaagatgggggaaaaagaatggtattttttttgccagagagaccggaaatatccgaccgggatgagaacgaaccgggcgactcagaccggttactggaaagcgaccgggaaagacaaggagattctcaagggaagaacggttctggctggtatgaagaaaacgctggttttttacaaaggaagagctcccaaaggtgaaaagaccaattgggtcatgcatgaatttcgactcgaacccaaattctttcagtttcttggttttcccaagcccattaaggctgattgggttgtatgtcgggtttttcacaagaacacaacgaacacggtcggagtagtgaaaaagattcaaacttctgatttttcttcttctctcccacctctaatagatcccacaactgctcatactccaatcagtggcagattcgataatggtgaagtcaactggaggttatcagtaccattcgataattatgcaaatgattaccattatcatcggcctttttcagcgacgaatactgcagtgacaatgatttcgtcgtacccatcgtctgtccccgacgacgaattcttctcatttgatcaactagacgtcggtggaacaatgtcaatggcggcggcgacgacaacaacaacaacaactatggagtgcaaaatagaacaagtttcatggtcaacgatgagcggtgtgacaccggagatatcatcgtcgattgacaacgaggcagctctcgagttctgggactactgaaaattgaaagtagatgttatgatcgaacaatggcgatgctttgttttaaatgggcatttcccatattgaacgtttaaacaatgattaattgattgctaattattattattttttttttttggttacatagtcctttttgggaaggaatattagaactttcatgggtttggtttgttgattgtattgatatgtagcaatgtgacattgtatatagcttctttatcttttattttaaccgttgcaaa

SEQ ID NO: 26 - RNA, 1964 nt, Arabidopsis thaliana
  source 1..1964: mol_type = unassigned RNA
taataatcattttttttcttataaccttcctctctatttttacaatttattttgttattagaagtggtagtggagtgaaaaaacaaatcctaagcagtcctaaccgatccccgaagctaaagattcttcaccttcccaaataaagcaaaacctagatccgacattgaaggaaaaaccttttagatccatctctgaaaaaaaaccaaccatgaagagagatcatcatcatcatcatcaagataagaagactatgatgatgaatgaagaagacgacggtaacggcatggatgagcttctagctgttcttggttacaaggttaggtcatcggaaatggctgatgttgctcagaaactcgagcagcttgaagttatgatgtctaatgttcaagaagacgatctttctcaactcgctactgagactgttcactataatccggcggagctttacacgtggcttgattctatgctcaccgaccttaatcctccgtcgtctaacgccgagtacgatcttaaagctattcccggtgacgcgattctcaatcagttcgctatcgattcggcttcttcgtctaaccaaggcggcggaggagatacgtatactacaaacaagcggttgaaatgctcaaacggcgtcgtggaaaccaccacagcgacggctgagtcaactcggcatgttgtcctggttgactcgcaggagaacggtgtgcgtctcgttcacgcgcttttggcttgcgctgaagctgttcagaaggagaatctgactgtggcggaagctctggtgaagcaaatcggattcttagctgtttctcaaatcggagctatgagaaaagtcgctacttacttcgccgaagctctcgcgcggcggatttaccgtctctctccgtcgcagagtccaatcgaccactctctctccgatactcttcagatgcacttctacgagacttgtccttatctcaagttcgctcacttcacggcgaatcaagcgattctcgaagcttttcaagggaagaaaagagttcatgtcattgatttctctatgagtcaaggtcttcaatggccggcgcttatgcaggctcttgcgcttcgacctggtggtcctcctgttttccggttaaccggaattggtccaccggcaccggataatttcgattatcttcatgaagttgggtgtaagctggctcatttagctgaggcgattcacgttgagtttgagtacagaggatttgtggctaacactttagctgatcttgatgcttcgatgcttgagcttagaccaagtgagattgaatctgttgcggttaactctgttttcgagcttcacaagctcttgggacgacctggtgcgatcgataaggttcttggtgtggtgaatcagattaaaccggagattttcactgtggttgagcaggaatcgaaccataatagtccgattttcttagatcggtttactgagtcgttgcattattactcgacgttgtttgactcgttggaaggtgtaccgagtggtcaagacaaggtcatgtcggaggtttacttgggtaaacagatctgcaacgttgtggcttgtgatggacctgaccgagttgagcgtcatgaaacgttgagtcagtggaggaaccggttcgggtctgctgggtttgcggctgcacatattggttcgaatgcgtttaagcaagcgagtatgcttttggctctgttcaacggcggtgagggttatcgggtggaggagagtgacggctgtctcatgttgggttggcacacacgaccgctcatagccacctcggcttggaaactctccaccaattagatggtggctcaatgaattgatctgttgaaccggttatgatgatagatttccgaccgaagccaaactaaatcctactgtttttccctttgtcacttgttaagatcttatctttcattatattaggtaattgaaaaatttctaaattactcacactggc

SEQ ID NO: 27 - RNA, 1556 nt, Lycopersicon esculentum
  source 1..1556: mol_type = mRNA
aaagaaaaaaggaatattgtgtgtttgcttttttttctgactagtagtattgctaactatgtattccattaaggatttgctgtgaaaaagcctgatatcagtaagcataaaactcgggagatcacttacacacacacacaccctcctaaaaaagagaagagagatttactgttaaacagaggtttttttccatttctttttttttttcagtgtgtgtgtgagagaaagagatgattttcataggcacaaacaaatagaaaggaacaaaatttagagtgaagaagaaagtgtgtgagagaataatggagggtggttctagtggaaatactagtacatcttgtttaatgatgatgggatatggagatcatgaaaacaacaacaacaacaatggaaatggtaatggaaatggaaatggaaatgtaacaatttgtgctcctccaatgatgatgatgatgcctcctcctcctccttctttaactaacaataacaatgcagaaacaagcaacaacaacatcctttttcttcctttcatggacaacaacaacaacaataatcctcaagaagacaacaactcttcttcttcttccatcaagtcaaagattatggctcatcctcactaccatcgtctcttgactgcttatctcaattgtcaaaagataggagctccgccagaagtggtggcaaggctagaggaaatatgtgccacgtcagcaacaatgggccgtagcagtagtagtagtggtggtggaatcattggagaagatcctgcactagatcagttcatggaggcttattgtgagatgctgacaaaatatgaacaagaactctcaaaacccttcaaggaagccatggtttttctttcaagaattgagtgtcagttcaaagctttaactcttgcacctaattcttctcatgaatctgctttgggcgaggcaatggatagaaatggatcatctgatgaagaggttgacgtgaataacagtttcatcgacccccaggctgaggatagagagctcaaaggtcaattgttgcgtaagtacagcggttacttgggaagccttaagcaggagttcatgaagaagaggaagaaaggcaagctgcctaaggaagcaaggcaacaattggtggattggtggcttagacatattaaatggccatatccatcggaatctcagaagcttgcactagctgaatcaacgggattggaccagaagcaaataaacaactggtttatcaatcaaagaaagaggcattggaaaccatcagaagatatgcagtttgttgtgatggatgctgctcatccacattactatatggataatgttcttgctaaccatttcccaatggatatgacaccctctctcctctgaattaagatttgtcattattaatatcaaggatgtttaattaatttgcatattacttgtgtgcatgtagtagtacaagctattgtgacacaatcaactttttattagaccaaatatataaagtgcttgtaatagatctttctattatcatctttaattatggaattaaatagtttgtacttgctaaaa

SEQ ID NO: 28 - RNA, 2735 nt, Solanum tuberosum
  source 1..2735: mol_type = mRNA
catgcagagataaaaatatagatcagtctgacaagaaggcaacttctcaaagcttagagagctaccacccgaagatagacagttagttacatgtactgttatagataaaaggagaaatccgaagaagaaagaattttttttgcagatatgtactatcaaggaacctcggataatactaatatacaagctgatcatcaacaacgtcataatcatgggaatagtaataataataatattcagacactttatttgatgaaccctaacaattatatgcaaggctacactacttctgacacacagcagcagcagcagttacttttcctgaattcttcaccagcagcaagcaacgcgctttgccatgcgaatatacaacacgcgccgctgcaacagcagcactttgtcggtgtgcctcttccggcagtaagtttgcacgatcagatcaatcatcatggacttttacagcgcatgtggaacaaccaagatcaatctcagcaggtgatagtaccatcgtcgacgggggtttctgccacgtcatgtggcgggatcaccacggacttggcgtctcaattggcgtttcagaggccgattccgacaccacaacaccgacagcagcaacaacagcaaggcggtctatctctaagcctttctcctcagctacaacagcaaattagtttcaataacaatatttcatcctcatcaccaaggacaaataatgttactattaggggaacattagatggaagttctagcaacatggttttaggctctaagtatctgaaagctgcacaagagcttcttgatgaagttgttaatattgttggaaaaagcatcaaaggagatgatcaaaagaaggataattcaatgaataaagaatcaatgcctttggctagtgatgtcaacactaatagttctggtggtggtgaaagtagcagcaggcagaaaaatgaagttgctgttgagcttacaactgctcaaagacaagaacttcaaatgaaaaaagccaagcttcttgccatgcttgaagaggtggagcaaaggtacagacagtaccatcaccaaatgcaaataattgtattatcatttgagcaagtagcaggaattggatcagccaaatcatacactcaattagctttgcatgcaatttcgaagcaattcagatgcctaaaggatgcaattgctgagcaagtaaaggcgacgagcaagagtttaggtgaagaggaaggcttgggagggaaaatcgaaggctcaagactcaaatttgtggaccatcatctaaggcaacaacgcgcgctgcaacagataggaatgatgcaaccaaatgcttggagaccccaaagaggtttacctgaaagagctgtctctgtccttcgtgcttggcttttcgagcattttcttcatccttacccaaaggattcagacaaaatcatgcttgctaagcaaacggggctaacaaggagccaggtgtctaactggttcataaatgctcgagttcgattatggaagccaatggtagaagaaatgtacttggaagaagtgaagaatcaagaacaaaacagtactaatacttcaggagataacaaaaacaaagagaccaatataagtgctccaaatgaagagaaacatccaattattactagcagcttattacaagatggtattactactactcaagcagaaatttctacctcaactatttcaacttcccctactgcaggtgcttcacttcatcatgctcacaatttctccttccttggttcattcaacatggataatactactactactgttgatcatattgaaaacaacgcgaaaaagcaaagaaatgacatgcacaagttttctccaagtagtattctttcatctgttgacatggaagccaaagctagagaatcatcaaataaagggtttactaatcctttaatggcagcatacgcgatgggagattttggaaggtttgatcctcatgatcaacaaatgaccgcgaattttcatggaaataatggtgtctctcttactttaggacttcctccttctgaaaacctagccatgccagtgagccaacaaaattacctttctaatgacttgggaagtaggtctgaaatggggagtcattacaatagaatgggatatgaaaacattgattttcagagtgggaataagcgatttccgactcaactattaccagattttgttacaggtaatctaggaacatgaataccagaaagtctcgtattgatagctgaaaagataaaaggaagttagggatactcttatattgtgtgaggccttctggcccaagtcggaggacccaatttgatacaacctatcataggagaaaagaagtggagactaaattaaagtaacaaaattttaaagcacactttctagtatatatacttcttttttttatagtatagaaaagaagagattttgtgctttagtgtatagatagagtctacttagtataggttatacttctagttccttgagaagattgatacaactagtagtattttttttcttttgggttggcttggagtactattttaagttattggaaactagctatagtaaatgttgtaaagttgtgatattgttcctctcaatttgcatataatttgaaatattttgtacctactagctagtctctaaattatgtttccattgcttgtaattgcaattttatttgaattttgtgctatcattattagattagcaaaaaaaaaaaaaaaaaa

SEQ ID NO: 29 - RNA, 72 nt, Arabidopsis thaliana
  source 1..72: mol_type = unassigned RNA
atcagagtggcgcagcggaagcgtggtgggcccataacccacaggtcccaggatcgaaacctggctctgata

SEQ ID NO: 30 - RNA, 71 nt, Arabidopsis thaliana
  source 1..71: mol_type = unassigned RNA
gcaccagtggtctagtggcatgatagtaccctgccacggtacagacccgggttcaattcccggctggtgca

SEQ ID NO: 31 - DNA, 699 bp, Cucurbita maxima
  source 1..699: mol_type = unassigned DNA
ggggcttcaaaagagaattaggtcacctcccagctcggtttcgacacgccatgccgagaaatcgtgaccctctagtcgtcgggagagtgatcggcgacgtcgtcgactcgttctcgaggtccatctcgattagggttgtttacgactcgagggaagttaacaatgggtgtgagctcaaaccctctcaagctgtcaacaagccaagagttgagattggtggcactgaccttcgcaccttcttcactttggttatggtggatcccgacgctcctagccctagcgatcccaatctaagagaatacttgcattggttagtgaccgatattccagctacaaccgaggcaacctttggacaagagatagtgtgctacgagaatccaagaccaacggtgggtatccaccgttttgtgctggtcttgttccggcagctcggaaggcaaacggtgtatgctcctgggtggcgccagaacttcaacaccagacactttgcagagctttacaatcttggttcgccagtcgccgccgtctatttcaattgccaaagggaaaatggctccggtggaaggagaagagccggcgatgaatgttcataaaaacacttcacttcacattatattatcaaccaatatattgtaataacatggttcacgtttctatctaatagattatatatttttaataagttcgtgaaaaaaaaaa

SEQ ID NO: 32 - DNA, 858 bp, Cucurbita maxima
  source 1..858: mol_type = unassigned DNA
gagacaattacgcatcttttcagctctctcacgtactaccatcctctcgacgccatgccgagagaccgtgaccctttggtcgttgggagagtcatcggcgacgttatcgactcgttcacgaagtccatttcgattagggctacttacaacaacagggaaattagcaatggctgtgagctcaaaccctctcaagttgtcaaccagccaagagttgagattggtggcactgaccttcgcaccttcttcactttggttatggtggatcctgatgctcctagccctagtgatcctaatctaagggaatacttgcattggttggtgactgatatcccagctacaactggagcgaactttggtcaagagatcgtgtgctatgagagcccaagacccacggtgggtatccatcgtcttgtgctggtgttgtttcgacagcttggaaggcaaacggtgtacgctcctgggtggcgccagaacttcaacacaagagactttgcagagctttacaatcttggcttgccggtggcagccgtttatttcaattgccaaagggaaagtgggtctggtggaaggagaagaacccaagatgatttctaagccccacttcacattaattagattaatattatagcccctatcatctattaatcctaccttgcttttagattaacctttattttgagtacacccatggatcataaataagcccaaaatgcattcctaatattgctcttatactcgtttcgtatgaatcactgtcttttcttctttgtttttcttgttcgagtgttcatgttgtgctttttttttcgtatgaatcaaagtagaagatcaagattcgaaaaaaaaaaaaaaaaaaaa

SEQ ID NO: 33 - DNA, 596 bp, Vitis vinifera
  source 1..596: mol_type = unassigned DNA
ccctcttgtattgtatcggtgaggtgtgtgtgatgcctagggaaagggatcctcttgttgttgggcgcgttgtcggggatgttctggacccctttctcaggtccatcactctgagggtgacctacaataatagagaagtagcaaatggctgtgagttcagaccctctcagctagtcagccaacctagggtggacattggaggggatgacttgaggaccttctatactttggttatggtggaccctgacgctccaagccccagtaatccgaacctaagggagtacttacattggttggtgactgatattccagcaactactggggcaaacttcggccaagagattgtgtgttatgagagcccacgcccaacagctgggattcatcgctttgtttttgtattgtttcgccaactgggtaggcagacagtgtatgcaccagggtggcgccaaaatttcaacactagggactttgctgagctttataatcttggtttgcctgttgctgctgtttattttaactgccaaagggagggcggctcgggtggtcgaagatcataatcaatggattttgtacgcaaccttgcgacttacaaaggc

SEQ ID NO: 34 - DNA, 525 bp, Malus x domestica
  source 1..525: mol_type = unassigned DNA
atgcctagggatagggacccccttgttgttggacgagtggttggtgatgttttagaccccttcacaaggtctgtttctctgagggtgacctacggtactaaggaggttaacaatggttgtgagctcaaaccttctgaagttgtccaacaacctagagctgatattggtggagacgatctcaggactttctacactctggtcatggtggatcctgatgcacccagcccaagtgaccccaacctaaaggaatatttgcattggttggttaccgatattccagcaactactgcggcaagcttcgggcaagagatcgtgtgttatgaaagtccacggccaacagtggggattcatcgctttgttttggtggtgtttcgccaattgggtaggcaaacggtgtatgctccgggatggcgccagaacttcaataccagagacttcgccgagctttataatcttggattaccggtgtctgtcgtctattttaactgccaaagggagggcggctccggtggaaggagaagataa

SEQ ID NO: 35 - DNA, 745 bp, Citrus unshiu
  source 1..745: mol_type = unassigned DNA
ggcacgaggaatagtcttactacttttgtaggctgtgtgtgtatttgtttgtgcttagtgttgttgagtgtttgtttgtgtttagtgttgttgatatgtctagcagggagagagatcctcttattgttggccgcgttgttggtgatgttcttgacaattttacaagaacaattccaatgaggattacctattcaaacaaggatgttaataatggccgtgagctcaaaccttctgaagttctgaaccagcctagggctgaaattggtggtgatgatcttaggacattttatactttggtaatggttgatcctgatgcaccaagcccaagtgaccccagccttagggagtatttgcattggttggtgactgatattccagcaaccacaggggccagctttggccaagagattgtgaactatgaaagccctaggccaacgatggggattcacaggtttgtctttgtgttgttccggcaacttgggaggcagactgtttatgcaccagggtggcgtcagaacttcagcacgagggattttgctgagctttacaatctgggacctccggtggccgctgtctacttcaactgccagagggagagcggatccggcggaaggcctgtcagacgatgatccatacatgcttaatttgatatcaaattacacacacacacacacacacacacacacacacacacacacacacacacacacactatttatatatatatatatatatatatatatat

SEQ ID NO: 36 - DNA, 534 bp, Lycopersicon esculentum
  source 1..534: mol_type = unassigned DNA
atgcctagagaacgtgatcctcttgttgttggtcgtgtggtaggggatgtattggaccctttcacaagaactattggcctaagagttatatatagagatagagaagttaataatggatgcgagcttaggccttcccaagttattaaccagccaagggttgaagttggaggagatgacctacgtacctttttcactttggttatggtggaccctgatgctccaagtccgagtgatccaaatctgagagaataccttcactggttggtcaccgatattccagctaccacaggttcaagttttgggcaagaaatagtgagctatgaaagtccaagaccatcaatgggaatacatcgatttgtatttgtattattcagacaattaggtcggcaaacagtgtatgctccaggatggcgtcagaatttcaacacaagagattttgcagaactttataatcttggtttacctgttgctgctgtctattttaattgtcaaagagagagtggcagtggtggacgtagaagatctgctgattga

SEQ ID NO: 37 - DNA, 525 bp, Populus tremula
  source 1..525: mol_type = unassigned DNA
atgtcaagggacagagatcctctgagcgttggccgtgttataggggacgtgctggaccccttcacaaagtctatcccgctcagggtcacctacaactccagagaggtcaacaatggttgcgagctcaaaccctctcaggttgccaaccagccgagggttgatattggcggggaagatctaaggaccttctacactctggttatggtggaccctgatgcacccagcccaagtgaccccagcctcagagaatatttgcattggttggtgactgatattccagcaacaacgggggcaagctttggccatgaaactgtgtgctatgagagcccgaggccgacgatggggattcatcggtttgttttcgtcttgttccggcaactgggcaggcaaactgtgtatgcccctgggtggcgccagaacttcaacaccagagactttgctgaggtctacaatcttggatcgccggtggctgctgtttatttcaactgccagagggagagtggctctggtggtaggaggcgataa

SEQ ID NO: 38 - DNA, 534 bp, Hordeum vulgare
  source 1..534: mol_type = unassigned DNA; sub_species = vulgare
atggccgggagggacagggatccgctggttgtcggcagggttgtgggggacgtgctggaccccttcgtccgaaccaccaacctcagggtgaccttcgggaacagggccgtgtccaacggctgcgagctcaagccgtccatggtcgcccagcagccgagggtggaggtgggcggcaatgagatgaggaccttctacacgctcgtgatggtagacccagatgctccaagtcctagcgaccccaaccttagagagtatctccactggttggtgacagatatcccgggtacaactggggcgtcgttcgggcaggaggtgatgtgctacgagagccctcgtccaaccatggggatccaccgcttcgtgctcgtgctcttccagcagctggggcggcagacggtgtacgcccccgggtggcgccagaacttcaacaccagggactttgccgagctctacaacctcggccagcccgttgccgccgtctacttcaactgccagcgcgaggccggctccggcggcaggaggatgtacaattga

SEQ ID NO: 39 - DNA, 537 bp, Hordeum vulgare
  source 1..537: mol_type = unassigned DNA; sub_species = vulgare
atggtggggagcagcatgcagcgcggggacccgctggtggtggggcgggtgatcggcgacgtggtggacccgttcgtgcggcgggtggcgctgcgggtcggctacgcgtccagggacgtggccaacggctgcgagctccggccgtccgccatcgccgaccagccgcgcgtcgaggtcggcggcccggacatgcgcaccttctacaccctggtgatggtggatccggatgctccaagccccagcgaccccagccttagggagtacttgcactggctggtcaccgacatcccggccacgacaggagtgtcttttggtaccgaggttgtgtgctacgagggcccgcggccggtgctcgggatccaccgactggtgttcctgctcttccagcaactcggccgacagacggtgtacgccccggggtggcggcagaacttcagcacccgcgactttgccgagctctacaacctcggcctgcccgtcgccgccgtctacttcaactgccagagggagaccggaaccggcgggagaaggatgtga

SEQ ID NO: 40 - DNA, 847 bp, Oryza sativa
  source 1..847: cultivar = Nipponbare; mol_type = unassigned DNA; note = Japonica Group
tgcaccacacacagttcagctagcagatcacctagctagatagctgcctctatcacagtatatttgctccctgcaacttgctgctgctgcaatagctagcagctgcagctagtaagcaaaactataaaccttcagggttttttgcaagatcgatggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggcaggctccggcggcaggagggtctacccctagctaacgatgatcccgatcgatctgctgcatgctcactatcatcatccagcatgctatacattgcaggttcagacaattgaaatgattctcgacacacaacatatatatgatggtgtaattaattatgcaattaaatagctgagcaaggctaaggt

SEQ ID NO: 41 - DNA, 866 bp, Oryza sativa
  source 1..866: mol_type = unassigned DNA; note = Japonica Group
cctgtcactgtttggctagcttaaccttcctgacatctatcctctggattgaacggcaggagatacctaagctagctagcaatctctatcgatctgtttgtttacatgttcagttaaaggttactgagaaatgcctagagtttttccggctagcttcataagttagtgggttagctgacctagattcaaagtctaatccttttatttatttgatattagatatcctaacgtttttagttagaggttattaatttgacatggccggcagcggcagggacgatcctcttgtggttggcaggattgtgggtgatgtgctggatccattcgtccggatcactaacctcagtgtcagctatggtgcaaggatcgtctccaatggctgcgagctcaagccgtccatggtgacccaacagcccagggtcgtggtcggtggcaatgacatgaggacgttctacacactcgtgatggtagacccggatgctccgagcccaagcaaccctaaccttagggagtatctacactggctggtcaccgatattcctggtaccactggagcaacatttgggcaagaggtgatgtgctacgagagcccaaggccaaccatggggatccaccggctggtgttcgtgctgttccagcagctggggcgtcagacggtgtacgcaccggggtggcgccagaacttcagcaccaggaacttcgccgagctctacaacctcggctcgccggtcgccaccgtctacttcaactgccagcgcgaggccggctccggcggcaggagggtctacccctagctagctacgcatgccacccggcctccatgcatgcagcagctatagctaagctgagacctgcctagctgtata

SEQ ID NO: 42 - DNA, 848 bp, Ipomoea nil
  source 1..848: mol_type = unassigned DNA
cacacacacacacatatatatacagagaaaggttagttttgatcgaggagctgagctagctaggatgcgaaggggaacagtagaccctttggtgttggggcgtgtgatcggagacgttgtggatccattcacgaggtccgttgagcttagggtggtttacaataacgaggtggatatcaggaatgggtgtgagatgaggccttctcagctcatcaacccacctagggttgaaatcggcggacacgatctccgtactttctacactctggttatggtggatcctgatgctccaagtccaacctctccaaccctgagggaatacctccactggttggtcactgatataccaggaactacaggagcaagcttcggcaatgaagcgatattctacgagcctccaaggccgtcaatgggaatccaccgttttgtgtttgtgcttttccggcaacttggccggcagacagtttatgcaccggtttggcgccagaatttcaacactcgaaactttgctgagatttacaatcttggtttgccagtggccgtcacttactttaacggccaaagggagggtggcaccggcggtcgatctccggcagagccctgggcagccgattaattaccctgctccttcccgttaatttcatgcatgcatgcatgctatctatagcataacatacatatagtatatatcataaataaataagaccacatgcattaacatgtttaattttcccatgaatatatgttaaagttgttctagaagaactacgtactccattatattaccctttatatatggcaatgaagatggtttcatctctatttagaagctaaaaaaaaaaaaaaaa

SEQ ID NO: 43 - DNA, 798 bp, Arabidopsis thaliana
  source 1..798: mol_type = unassigned DNA
tttattgagatacttgagatccaagataaatatgtctttagtcgtagagatcctcttgtggtcggcagtgttgttggagatgttcttgatcctttcacgaggttggtctctcttaaggtcacttatggccatagagaggttactaatggcttggatctaaggccttctcaagttctgaacaaaccaatagtggagattggaggagacgacttcagaaatttctacaccttggttatggtggatccagatgtgccgagtccaagcaaccctcaccaacgagaatatctccactggttggtgactgatatacctgccaccactggaaatgcctttggcaatgaggtggtgtgctacgagagtccacgtcccccctcgggaattcatcgtattgtgttggtattgttccggcaactcggaagacaaacggtttatgcaccggggtggcgccaacagttcaacactcgtgagtttgctgagatctacaatcttggtcttcctgtggctgcctcttacttcaactgccagagggagaatggctgtgggggaagaagaacgtagatgcgtacctacttacgttaactaataatctaatcgtataatattcccttaatgaagtatttaagcatctatgtcaatgtaataagaatttaaagatacgagctaaaaaaaatgatgcatatgctgacatcgatgtaaagtagtttacacttttaatgtaataactaggttttaacccgcggtacaccgcgagactattttgtttttttaagaataaaaatataatttgtttagtcgatt

SEQ ID NO: 44 - DNA, 519 bp, Glycine max
  source 1..519: cultivar = Toyoharuka; mol_type = unassigned DNA
atggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggttatggtggatgcagatgcacctagccctagcaaccctgtcttgagggaataccttcactggatggtgacagatattccagctaccacaaatgcaagctttgggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 45 - DNA, 560 bp, Zea mays
  source 1..560: mol_type = unassigned DNA
agagcacatccgtagtgtgtgcatgcatcacagtcacacacacacagcagaagaagaagaaaccgaacgagggtttagctagcaaaataaacagaagcaagcaagctagctagagctaaggatcgagatcgagatcgaccgaccgacgacgatcagctagcatggcgcgcttcgtggatccgctggtggtggggcgggtgatcggcgaggtggtggacctgttcgtgccttccatctccatgaccgtcgcctatgatggccccaaggacatcagcaacggctgcctcctcaagccgtccgccaccgccgcgccgccgctcgtccgcatctccggccgccgcaacgacctctacacgctgatcatgacggaccccgatgcgcctagccccagcaacccgaccatgagggagtacctccactggatagtgattaacataccaggaggaacagatgctactaaaggtgaggaggtggtggagtacatgggcccgcggccgccggtgggtatccaccgctacgtgctggtgctgttcgagcagaagacgcgcgtgcacgcggag

SEQ ID NO: 46 - DNA, 1069 bp, Zea mays
  source 1..1069: mol_type = unassigned DNA
tggcaaaaacccagcgctttgtgccgccgccgtccgccggcccctctgcccttgtacgcgcacctagacacatcgtcatcgatcatcacacgcaatcgacacaagaagttaataaacagcccaaggacgcagagatcagctgatcgagaaggacttgtactactactcagtattgtcgtcacatgcacatatatgtacataaagagctagctacctgagctctacccaaggtcgcgttgatcgatcgatcatggcgcggttcgtggacccgctggtggtggggcgggtgatcggcgaggtggtggacctgttcgtgccctccgtctccatgaccgtcgcctatggccccaaagacatcagcaacggctgcctcctcaagccgtccgccaccgccgcgccgccgctcgtccgcatctccggccgccgcgacgacctctacacgctgatcatgacggacccagatgcgcctagccccagcgacccgaccatgagggagtacctccactggatagtgactaacataccaggaggaacggatgcaaacaaaggtgaggaggtggtggagtacatgggcccgcggccgccggtcggaatccaccgctacgtgctggtgctgttcgagcagaagacgcgtgtgcacgcggagggtcccggtgagcgcgccaacttcaacacacgcgcgttcgcggcggcgcacgagctcggcctccccaccgccgtcgtgtacttcaacgcgcagaaagagccggccaaccaccgccgccgctagctagtagctccaacaagggcgcgccagctgagctgcgtgcgtgcaacccaccacacagccgccggcgaaggctgcctatatgaccggcgaataaaaagtcttactgcaccgtccgtaagcgtactctctgttggtatatgcttgtcttcaggctcttgagtctatctacttaaatgtggttaccactgagtaatagaagcagttggcgcttcgatcgatcattctaatatccgtacgtgtcaatcattcctgtttccatcatcttgcatttgaagacgcattggttctacaccaaggtgt

SEQ ID NO: 47 - DNA, 1288 bp, Cucurbita maxima
  source 1..1288: mol_type = unassigned DNA
gactttttattcaacaatctctctctctctctctcaacttccgatcaagtctctccgccgtcttttcaccggagctgacaattccgatcattttttgcttcccttaaatttccggcatggaggaaccaccgccaaacgccttggatttgccccctggcttcagattccaccccaccgacgaggagatcgtcacttattacctgatacataagatcaccgacgccgccttcactgccaccgccatcggagaagctgacctgaataagtgtgaaccttgggatttgccacataaagctaagatgggggaaaaagaatggtattttttttgccagagagaccggaaatatccgaccgggatgagaacgaaccgggcgactcagaccggttactggaaagcgaccgggaaagacaaggagattctcaagggaagaacggttctggctggtatgaagaaaacgctggttttttacaaaggaagagctcccaaaggtgaaaagaccaattgggtcatgcatgaatttcgactcgaacccaaattctttcagtttcttggttttcccaagcccattaaggctgattgggttgtatgtcgggtttttcacaagaacacaacgaacacggtcggagtagtgaaaaagattcaaacttctgatttttcttcttctctcccacctctaatagatcccacaactgctcatactccaatcagtggcagattcgataatggtgaagtcaactggaggttatcagtaccattcgataattatgcaaatgattaccattatcatcggcctttttcagcgacgaatactgcagtgacaatgatttcgtcgtacccatcgtctgtccccgacgacgaattcttctcatttgatcaactagacgtcggtggaacaatgtcaatggcggcggcgacgacaacaacaacaacaactatggagtgcaaaatagaacaagtttcatggtcaacgatgagcggtgtgacaccggagatatcatcgtcgattgacaacgaggcagctctcgagttctgggactactgaaaattgaaagtagatgttatgatcgaacaatggcgatgctttgttttaaatgggcatttcccatattgaacgtttaaacaatgattaattgattgctaattattattattttttttttttggttacatagtcctttttgggaaggaatattagaactttcatgggtttggtttgttgattgtattgatatgtagcaatgtgacattgtatatagcttctttatcttttattttaaccgttgcaaa

SEQ ID NO: 48 - DNA, 1964 bp, Arabidopsis thaliana
  source 1..1964: mol_type = unassigned DNA
taataatcattttttttcttataaccttcctctctatttttacaatttattttgttattagaagtggtagtggagtgaaaaaacaaatcctaagcagtcctaaccgatccccgaagctaaagattcttcaccttcccaaataaagcaaaacctagatccgacattgaaggaaaaaccttttagatccatctctgaaaaaaaaccaaccatgaagagagatcatcatcatcatcatcaagataagaagactatgatgatgaatgaagaagacgacggtaacggcatggatgagcttctagctgttcttggttacaaggttaggtcatcggaaatggctgatgttgctcagaaactcgagcagcttgaagttatgatgtctaatgttcaagaagacgatctttctcaactcgctactgagactgttcactataatccggcggagctttacacgtggcttgattctatgctcaccgaccttaatcctccgtcgtctaacgccgagtacgatcttaaagctattcccggtgacgcgattctcaatcagttcgctatcgattcggcttcttcgtctaaccaaggcggcggaggagatacgtatactacaaacaagcggttgaaatgctcaaacggcgtcgtggaaaccaccacagcgacggctgagtcaactcggcatgttgtcctggttgactcgcaggagaacggtgtgcgtctcgttcacgcgcttttggcttgcgctgaagctgttcagaaggagaatctgactgtggcggaagctctggtgaagcaaatcggattcttagctgtttctcaaatcggagctatgagaaaagtcgctacttacttcgccgaagctctcgcgcggcggatttaccgtctctctccgtcgcagagtccaatcgaccactctctctccgatactcttcagatgcacttctacgagacttgtccttatctcaagttcgctcacttcacggcgaatcaagcgattctcgaagcttttcaagggaagaaaagagttcatgtcattgatttctctatgagtcaaggtcttcaatggccggcgcttatgcaggctcttgcgcttcgacctggtggtcctcctgttttccggttaaccggaattggtccaccggcaccggataatttcgattatcttcatgaagttgggtgtaagctggctcatttagctgaggcgattcacgttgagtttgagtacagaggatttgtggctaacactttagctgatcttgatgcttcgatgcttgagcttagaccaagtgagattgaatctgttgcggttaactctgttttcgagcttcacaagctcttgggacgacctggtgcgatcgataaggttcttggtgtggtgaatcagattaaaccggagattttcactgtggttgagcaggaatcgaaccataatagtccgattttcttagatcggtttactgagtcgttgcattattactcgacgttgtttgactcgttggaaggtgtaccgagtggtcaagacaaggtcatgtcggaggtttacttgggtaaacagatctgcaacgttgtggcttgtgatggacctgaccgagttgagcgtcatgaaacgttgagtcagtggaggaaccggttcgggtctgctgggtttgcggctgcacatattggttcgaatgcgtttaagcaagcgagtatgcttttggctctgttcaacggcggtgagggttatcgggtggaggagagtgacggctgtctcatgttgggttggcacacacgaccgctcatagccacctcggcttggaaactctccaccaattagatggtggctcaatgaattgatctgttgaaccggttatgatgatagatttccgaccgaagccaaactaaatcctactgtttttccctttgtcacttgttaagatcttatctttcattatattaggtaattgaaaaatttctaaattactcacactggc

SEQ ID NO: 49 - DNA, 1556 bp, Lycopersicon esculentum
  source 1..1556: mol_type = unassigned DNA
aaagaaaaaaggaatattgtgtgtttgcttttttttctgactagtagtattgctaactatgtattccattaaggatttgctgtgaaaaagcctgatatcagtaagcataaaactcgggagatcacttacacacacacacaccctcctaaaaaagagaagagagatttactgttaaacagaggtttttttccatttctttttttttttcagtgtgtgtgtgagagaaagagatgattttcataggcacaaacaaatagaaaggaacaaaatttagagtgaagaagaaagtgtgtgagagaataatggagggtggttctagtggaaatactagtacatcttgtttaatgatgatgggatatggagatcatgaaaacaacaacaacaacaatggaaatggtaatggaaatggaaatggaaatgtaacaatttgtgctcctccaatgatgatgatgatgcctcctcctcctccttctttaactaacaataacaatgcagaaacaagcaacaacaacatcctttttcttcctttcatggacaacaacaacaacaataatcctcaagaagacaacaactcttcttcttcttccatcaagtcaaagattatggctcatcctcactaccatcgtctcttgactgcttatctcaattgtcaaaagataggagctccgccagaagtggtggcaaggctagaggaaatatgtgccacgtcagcaacaatgggccgtagcagtagtagtagtggtggtggaatcattggagaagatcctgcactagatcagttcatggaggcttattgtgagatgctgacaaaatatgaacaagaactctcaaaacccttcaaggaagccatggtttttctttcaagaattgagtgtcagttcaaagctttaactcttgcacctaattcttctcatgaatctgctttgggcgaggcaatggatagaaatggatcatctgatgaagaggttgacgtgaataacagtttcatcgacccccaggctgaggatagagagctcaaaggtcaattgttgcgtaagtacagcggttacttgggaagccttaagcaggagttcatgaagaagaggaagaaaggcaagctgcctaaggaagcaaggcaacaattggtggattggtggcttagacatattaaatggccatatccatcggaatctcagaagcttgcactagctgaatcaacgggattggaccagaagcaaataaacaactggtttatcaatcaaagaaagaggcattggaaaccatcagaagatatgcagtttgttgtgatggatgctgctcatccacattactatatggataatgttcttgctaaccatttcccaatggatatgacaccctctctcctctgaattaagatttgtcattattaatatcaaggatgtttaattaatttgcatattacttgtgtgcatgtagtagtacaagctattgtgacacaatcaactttttattagaccaaatatataaagtgcttgtaatagatctttctattatcatctttaattatggaattaaatagtttgtacttgctaaaa

SEQ ID NO: 50 - DNA, 2735 bp, Solanum tuberosum
  source 1..2735: mol_type = unassigned DNA
catgcagagataaaaatatagatcagtctgacaagaaggcaacttctcaaagcttagagagctaccacccgaagatagacagttagttacatgtactgttatagataaaaggagaaatccgaagaagaaagaattttttttgcagatatgtactatcaaggaacctcggataatactaatatacaagctgatcatcaacaacgtcataatcatgggaatagtaataataataatattcagacactttatttgatgaaccctaacaattatatgcaaggctacactacttctgacacacagcagcagcagcagttacttttcctgaattcttcaccagcagcaagcaacgcgctttgccatgcgaatatacaacacgcgccgctgcaacagcagcactttgtcggtgtgcctcttccggcagtaagtttgcacgatcagatcaatcatcatggacttttacagcgcatgtggaacaaccaagatcaatctcagcaggtgatagtaccatcgtcgacgggggtttctgccacgtcatgtggcgggatcaccacggacttggcgtctcaattggcgtttcagaggccgattccgacaccacaacaccgacagcagcaacaacagcaaggcggtctatctctaagcctttctcctcagctacaacagcaaattagtttcaataacaatatttcatcctcatcaccaaggacaaataatgttactattaggggaacattagatggaagttctagcaacatggttttaggctctaagtatctgaaagctgcacaagagcttcttgatgaagttgttaatattgttggaaaaagcatcaaaggagatgatcaaaagaaggataattcaatgaataaagaatcaatgcctttggctagtgatgtcaacactaatagttctggtggtggtgaaagtagcagcaggcagaaaaatgaagttgctgttgagcttacaactgctcaaagacaagaacttcaaatgaaaaaagccaagcttcttgccatgcttgaagaggtggagcaaaggtacagacagtaccatcaccaaatgcaaataattgtattatcatttgagcaagtagcaggaattggatcagccaaatcatacactcaattagctttgcatgcaatttcgaagcaattcagatgcctaaaggatgcaattgctgagcaagtaaaggcgacgagcaagagtttaggtgaagaggaaggcttgggagggaaaatcgaaggctcaagactcaaatttgtggaccatcatctaaggcaacaacgcgcgctgcaacagataggaatgatgcaaccaaatgcttggagaccccaaagaggtttacctgaaagagctgtctctgtccttcgtgcttggcttttcgagcattttcttcatccttacccaaaggattcagacaaaatcatgcttgctaagcaaacggggctaacaaggagccaggtgtctaactggttcataaatgctcgagttcgattatggaagccaatggtagaagaaatgtacttggaagaagtgaagaatcaagaacaaaacagtactaatacttcaggagataacaaaaacaaagagaccaatataagtgctccaaatgaagagaaacatccaattattactagcagcttattacaagatggtattactactactcaagcagaaatttctacctcaactatttcaacttcccctactgcaggtgcttcacttcatcatgctcacaatttctccttccttggttcattcaacatggataatactactactactgttgatcatattgaaaacaacgcgaaaaagcaaagaaatgacatgcacaagttttctccaagtagtattctttcatctgttgacatggaagccaaagctagagaatcatcaaataaagggtttactaatcctttaatggcagcatacgcgatgggagattttggaaggtttgatcctcatgatcaacaaatgaccgcgaattttcatggaaataatggtgtctctcttactttaggacttcctccttctgaaaacctagccatgccagtgagccaacaaaattacctttctaatgacttgggaagtaggtctgaaatggggagtcattacaatagaatgggatatgaaaacattgattttcagagtgggaataagcgatttccgactcaactattaccagattttgttacaggtaatctaggaacatgaataccagaaagtctcgtattgatagctgaaaagataaaaggaagttagggatactcttatattgtgtgaggccttctggcccaagtcggaggacccaatttgatacaacctatcataggagaaaagaagtggagactaaattaaagtaacaaaattttaaagcacactttctagtatatatacttcttttttttatagtatagaaaagaagagattttgtgctttagtgtatagatagagtctacttagtataggttatacttctagttccttgagaagattgatacaactagtagtattttttttcttttgggttggcttggagtactattttaagttattggaaactagctatagtaaatgttgtaaagttgtgatattgttcctctcaatttgcatataatttgaaatattttgtacctactagctagtctctaaattatgtttccattgcttgtaattgcaattttatttgaattttgtgctatcattattagattagcaaaaaaaaaaaaaaaaaa

SEQ ID NO: 51 - DNA, 72 bp, Arabidopsis thaliana
  source 1..72: mol_type = unassigned DNA
atcagagtggcgcagcggaagcgtggtgggcccataacccacaggtcccaggatcgaaacctggctctgata

SEQ ID NO: 52 - DNA, 71 bp, Arabidopsis thaliana
  source 1..71: mol_type = unassigned DNA
gcaccagtggtctagtggcatgatagtaccctgccacggtacagacccgggttcaattcccggctggtgca

SEQ ID NO: 53 - protein, 1230 aa, Francisella tularensis
  source 1..1230: note = subsp. novicida (strain U112)
ISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 54 - RNA, 38 nt, Francisella tularensis
  source 1..38: mol_type = unassigned RNA; note = subsp. novicida (strain U112)
gggtctaagaactttaaataatttctactgttgtacat

SEQ ID NO: 55 - protein, 1228 aa, unidentified
  source 1..1228: note = Lachnospiraceae bacterium ND2006
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 56 - RNA, 36 nt, unidentified
  source 1..36: mol_type = unassigned RNA; note = Lachnospiraceae bacterium ND2006
gtttcaaagattaaataatttctactaagtgtagat

SEQ ID NO: 57 - protein, 707 aa, synthetic construct
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 58 - RNA, 36 nt, synthetic construct
  source 1..36: mol_type = other RNA
ggagagatctcaaacgattgctcgattagtcgagac

SEQ ID NO: 59 - protein, 757 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 60 - RNA, 36 nt, synthetic construct
  source 1..36: mol_type = other RNA
gtcggaacgctcaacgattgcccctcacgaggggac

SEQ ID NO: 61 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 62 - RNA, 36 nt, synthetic construct
  source 1..36: mol_type = other RNA
accaaaacgactattgattgcccagtacgctgggac